(19) United States
(12) Patent Application Publication
(10) Pub. No.: US 2025/0104848 A1
(43) Pub. Date: Mar. 27, 2025

(54) BADGE AND PATIENT SUPPORT APPARATUS COMMUNICATION SYSTEM

(71) Applicant: Stryker Corporation, Kalamazoo, MI (US)

(72) Inventors: Krishna Sandeep Bhimavarapu, Kalamazoo, MI (US); Anish Paul, Portage, MI (US); Jerald A. Trepanier, Augusta, MI (US); Madhu Thomas, London, Ontario (CA); Madhu Sandeep Thota, Portage, MI (US); Celso Henrique Farnese Pires Pereira, Portage, MI (US); Christopher P. Alvarez, Kalamazoo, MI (US); Richard C. Mayoras, Jr., Kalamazoo, MI (US); Kirby M. Neihouser, Portage, MI (US); William Dwight Childs, Plainwell, MI (US); Megan Sara Bair, Portage, MI (US); Ramsey H. Othman, Portage, MI (US); Nicholas S. Brajak, Portage, MI (US)

(21) Appl. No.: 18/725,390

(22) PCT Filed: Jun. 28, 2023
(86) PCT No.: PCT/US2023/026462

§ 371 Date: Jun. 28, 2024

Related U.S. Application Data
(60) Provisional application No. 63/356,061, filed on Jun. 28, 2022.
(60) Provisional application No. 63/356,065, filed on Jun. 28, 2022.
(60) Provisional application No. 63/356,238, filed on Jun. 28, 2022.
(60) Provisional application No. 63/357,363, filed on Jun. 30, 2022.

(51) Int. Cl. G16H 40/20; A61G 7/05; G16H 10/60; G16H 40/67

(52) U.S. Cl. CPC G16H 40/20; A61G 7/05; G16H 10/60; G16H 40/67

(57) ABSTRACT

A patient support apparatus includes a patient support surface, a plurality of ultra-wideband transceivers, and a controller. The controller is adapted to perform the following: use the ultra-wideband transceivers to automatically determine a position of the badge relative to the patient support apparatus, to receive a badge identifier from the badge; determine if the badge is positioned inside or outside of a volume of space; and, if the badge is positioned inside the volume of space, transmit the badge identifier to a server using the network transceiver. The controller and/or server may also, or alternatively, determine an identity of the user associated with the badge, determine an identity of the patient, automatically transmit at least a portion of a care plan to the patient support apparatus, record data indicating who implemented one or more functions on the patient support apparatus, and/or forward a message to a nearest badge.

### CROSS-REFERENCE TO RELATED APPLICATIONS

This application claims priority to the following U.S. provisional patent applications: Ser. No. 63/356,065 filed Jun. 28, 2022, by inventors Jerald Trepanier et al. and entitled BADGE AND PATIENT SUPPORT APPARATUS COMMUNICATION SYSTEM; Ser. No. 63/356,061 filed Jun. 28, 2022, by inventors Krishna Sandeep Bhimavarapu et al. and entitled BADGE AND PATIENT SUPPORT APPARATUS COMMUNICATION SYSTEM; Ser. No. 63/356,238 filed Jun. 28, 2022, by inventors Madhu Sandeep Thota et al. and entitled BADGE AND PATIENT SUPPORT APPARATUS COMMUNICATION SYSTEM; and Ser. No. 63/357,363 filed Jun. 30, 2022, by inventors Madhu Sandeep Thota et al. and entitled BADGE AND PATIENT SUPPORT APPARATUS COMMUNICATION SYSTEM, the complete disclosures of all of which are incorporated herein by reference.

### BACKGROUND

The present disclosure relates to patient support apparatuses, such as beds, cots, stretchers, recliners, or the like. More specifically, the present disclosure relates to patient support apparatuses that communicate with one or more badges and/or other devices that are separate from the patient support apparatus itself.

### SUMMARY

According to the various aspects described herein, the present disclosure is directed to a patient support apparatus system that monitors the positions of badges worn by one or more different types of users, such as, but not limited to, caregivers, service technicians, cleaners, transportation assistants, etc. The system may include one or more servers and one more patient support apparatuses adapted to communicate with the server(s) and the badges. The patient support apparatuses may use the position of the badges to perform one or more automatic tasks, such as, but not limited to, displaying a patient's care plan when a caregiver is in the room with the patient; recording and/or validating tasks performed by healthcare personnel; determining identities of personnel wearing the badges; granting different access to the patient support apparatus functions based on user roles; notifying a nearest caregiver if a patient support apparatus is in an undesired state; logging in caregivers to a badge management server; issuing reminders to personnel to perform tasks; and other tasks. The workload of healthcare personnel is thereby reduced when the system automatically performs one or more of these tasks. These and other aspects of the present disclosure will be apparent to a person of ordinary skill light of the following written description and accompanying drawings.

According to a first aspect of the present disclosure, a patient support apparatus is provided that includes a support surface adapted to support a patient, a plurality of ultra-wideband transceivers, a network transceiver adapted to communicate with a healthcare facility computer network, and a controller. The controller is adapted to use radio frequency (RF) communication between the plurality of ultra-wideband transceivers and a badge to determine a position of the badge relative to the patient support apparatus. The controller is further adapted to receive a badge identifier from the badge; to determine if the badge is positioned inside or outside of a volume of space; and, if the badge is positioned inside the volume of space, to transmit the badge identifier to a server using the network transceiver.

According to a second aspect of the present disclosure, a patient support apparatus system is provided that includes a patient support apparatus and a server. The patient support apparatus includes a support surface adapted to support a patient; a plurality of ultra-wideband transceivers; a network transceiver adapted to communicate with the server; and a controller. The controller is adapted to use radio frequency (RF) communication between the plurality of ultra-wideband transceivers and a badge worn by a user to determine a position of the badge relative to the patient support apparatus; to receive a badge identifier from the badge; to determine if the badge is positioned inside or outside of a volume of space; and to transmit the badge identifier to the server using the network transceiver if the badge is inside the volume of space. The server is adapted to use the badge identifier to determine an identity of the user associated with the badge.

According to a third aspect of the present disclosure, a patient support apparatus system is provided that includes a patient support apparatus and a server. The patient support apparatus includes a support surface adapted to support a patient; a plurality of ultra-wideband transceivers; a network transceiver adapted to communicate with the server; and a controller. The controller is adapted to use radio frequency (RF) communication between the plurality of ultra-wideband transceivers and a badge worn by a user to determine a position of the badge relative to the patient support apparatus. The controller is further adapted to use RF communications between the plurality of ultra-wideband transceivers and a fixed locator to determine a position of the fixed locator relative to the patient support apparatus; to receive a badge identifier from the badge; to receive a locator identifier from the fixed locator; to determine if the badge is positioned inside or outside of a volume of space; to determine if the patient support apparatus is within a threshold distance of the fixed locator; to transmit the badge identifier to the server using the network transceiver if the badge is inside the volume of space; and to transmit the locator identifier to the server if the patient support apparatus is within the threshold distance of the fixed locator. The server is adapted to use the locator identifier to determine an identity of the patient, and to automatically transmit at least a portion of a care plan to the patient support apparatus in response to the badge being inside the volume of space.

According to a fourth aspect of the present disclosure, a patient support apparatus system is provided that includes a patient support apparatus and a server. The patient support apparatus includes a support surface adapted to support a patient; a plurality of ultra-wideband transceivers; a network transceiver adapted to communicate with the server; a first control adapted to control a first function of the patient support apparatus; and a controller. The controller is adapted to use radio frequency (RF) communication between the plurality of ultra-wideband transceivers and a badge worn by a user to determine a position of the badge relative to the patient support apparatus; to receive a badge identifier from the badge; to determine if the badge is positioned inside or outside of a volume of space; to detect when the first control is activated and the badge is positioned inside of the volume of space; and to transmit both the badge identifier and a function identifier to the server, wherein the function identifier identifies the first function. The server is adapted to perform at least one of the following: (i) record data indicating that the first function was implemented by the user with the badge identifier, or (ii) send a validation message to an electronic medical records server indicating that the user with the badge identifier implemented the first function.

According to a fifth aspect of the present disclosure, a patient support apparatus system is provided that includes a support surface adapted to support a patient; a plurality of ultra-wideband transceivers; a network transceiver adapted to communicate with the server; a first control adapted to control a first function of the patient support apparatus; and a controller adapted to use radio frequency (RF) communication between the plurality of ultra-wideband transceivers and a badge worn by a user to determine a position of the badge relative to the patient support apparatus. The controller is also adapted to determine if the badge is positioned inside or outside of a volume of space; to monitor a plurality of conditions of the patient support apparatus in order to determine if any of the conditions are in an undesired state; and, if the badge is positioned outside the volume of space while at least one of the conditions is in an undesired state, to automatically send a first message to the server. The server is adapted to select a badge and to send a second message to the selected badge indicating that a condition of the patient support apparatus is in an undesired state.

According to a sixth aspect of the present disclosure, a patient support apparatus is provided that includes a support surface adapted to support a patient thereon, a plurality of ultra-wideband transceivers, a network transceiver adapted to communicate with a healthcare facility computer network, and a controller. The controller is adapted to use radio frequency (RF) communication between the plurality of ultra-wideband transceivers and a badge to determine a position of the badge relative to the patient support apparatus, to receive a badge identifier from the badge, to determine if the badge is positioned inside or outside of a volume of space, to determine if a condition of the patient support apparatus is in an undesired state, and, if the badge is positioned inside the volume of space and a condition of the patient support apparatus is in the undesired state, to transmit a notification message to a person associated with the badge indicating that the patient support apparatus is in the undesired state.

According to still other aspects of the present disclosure, the controller may further be adapted to not transmit the notification message to the person associated with the badge if the badge is positioned outside of the volume of space.

In some aspects, the undesired state includes at least one of the following: a scale system onboard the patient support apparatus needing to be zeroed; or a power cord of the patient support apparatus not being plugged into an electrical outlet.

The controller, in some aspects, is adapted to transmit the notification message to the badge.

The controller, in some aspects, is adapted to transmit the notification message to a portable electronic device of the person associated with the badge.

The patient support apparatus, in some aspects, further includes a Bluetooth transceiver, and the controller is further adapted to transmit the notification message directly to the portable electronic device using the Bluetooth transceiver.

In some aspects, the controller is adapted to transmit the notification message to the person associated with badge using the network transceiver.

In some aspects, the controller may be further adapted to not transmit the badge identifier to the server if the badge is positioned outside of the volume of space.

In some aspects, the patient support apparatus further includes a first control that performs a first function when activated by a user, and wherein the controller is further adapted to transmit a validation message to the server validating that the first function was performed by the user if the badge is positioned inside the volume of space when the first control is activated.

In some aspects, the server is further adapted to transmit validation information to an electronic medical records server in response to receiving the validation message.

The first function, in some aspects, includes one or more of the following: changing a state of an exit detection system onboard the patient support apparatus; measuring a weight of the patient; performing a mattress therapy on the patient; changing a state of a mattress; changing a state of a brake onboard the patient support apparatus; changing a state of a siderail onboard the patient support apparatus; changing a height of the support surface; changing a reminder provided by the controller; zeroing a scale onboard the patient support apparatus; configuring a component onboard the patient support apparatus; changing a setting of the patient support apparatus; changing information stored in the patient support apparatus; or performing maintenance work on the patient support apparatus.

The patient support apparatus, in some aspects, further includes a display and a control panel, and the controller is adapted to allow a user to access a first set of screens on the display if the badge identifier corresponds to a first class, and to allow the user to access a second set of screens on the display if the badge identifier corresponds to a second class. The first set of screens includes at least one screen not in the second set of screens.

The controller, in some aspects, is adapted to display at least a portion of a care plan for the patient if the badge identifier corresponds to the first class, and to not display any portion of the care plan for the patient if the badge identifier correspond to the second class.

The controller, in some aspects, is adapted to monitor a plurality of conditions of the patient support apparatus and to determine if any of the conditions are not in a desired state.

In some aspects, the controller is further adapted to automatically send a message to the badge if the badge is positioned inside the volume of space, wherein the message indicates that a condition of the patient support apparatus is in an undesired state.

In some aspects, the controller is further adapted to use RF communication between the plurality of ultra-wideband transceivers and a fixed locator to determine a position of the patient support apparatus relative to the fixed locator.

The controller, in some aspects, is further adapted to receive a locator identifier from the fixed locator, to determine if the patient support apparatus is positioned within a threshold distance of the fixed locator, and to forward the locator identifier to the server if the patient support apparatus is positioned within the threshold distance.

The patient support apparatus, in some aspects, further includes a microphone positioned onboard the patient support apparatus. The microphone is adapted to convert sounds of the patient's voice to audio signals, and the controller is further adapted to transmit the audio signals to the fixed locator if the fixed locator is positioned within the threshold distance, and to not transmit the audio signals to the fixed locator if the fixed locator is positioned outside of the threshold distance.

The patient support apparatus, in some aspects, further includes a memory in which locations of each of the plurality of ultra-wideband transceivers are stored.

The controller, in some aspects, is further adapted to use the stored locations of the plurality of ultra-wideband transceivers to determine whether the badge is positioned inside or outside of the volume of space.

The controller, in some aspects, is further adapted to use radio frequency (RF) communication between the plurality of ultra-wideband transceivers and an device to determine a position of the device relative to the patient support apparatus; to determine if the device is positioned inside or outside of a second volume of space; to automatically disassociate the device from the patient support apparatus if the device is positioned outside of the second volume of space; and to automatically associate the device with the patient support apparatus if the device is positioned inside the second volume of space.

In some aspects, the controller is further adapted to perform at least one of the following when the device is associated with the patient support apparatus: (a) forward data received from the device to an off-board recipient, or (b) display data received from the device on a display of the patient support apparatus.

The controller, in some aspects, is further adapted to perform neither (a) nor (b) when the device is disassociated from the patient support apparatus.

The server, in some aspects, is further configured to transmit the identity of the user to a second server.

The second server, in some aspects, is adapted to maintain an association between badge identifiers and user identities.

The server, in some aspects, is further adapted to transmit validation information to an electronic medical records server in response to receiving the validation message.

In some aspects, the controller is further adapted to transmit a function identifier to the server if the badge is positioned inside the volume of space when the first control is activated. The function identifier identifies the first function.

In some aspects, the server is adapted to use both the badge identifier and the locator identifier to determine an identity of the user associated with the badge.

The server, in some aspects, is adapted to transmit at least a portion of a care plan to the patient support apparatus in response to receiving the badge identifier.

The controller, in some aspects, is further adapted to display the at least a portion of the care plan on a display coupled to the patient support apparatus when the badge is positioned within the volume of space, and to automatically cease displaying the at least a portion of the care plan on the display in response to the badge moving outside the volume of space.

The server, in some aspects, is further adapted to use to the locator identifier to determine a patient identity of the patient assigned to the patient support apparatus, and to use the patient identity to determine the at least a portion of the care plan.

In some aspects, the server is adapted to communicate with an electronic medical records server to determine the at least a portion of the care plan.

In some aspects, the controller is further adapted to determine whether the badge identifier belongs a first class of users or a second class of users and, if the badge identifier belongs to the first class, the controller is further adapted to automatically issue a first reminder to the user if the badge identifier belongs to the first class and the badge moves outside of the volume of space before a first function on the patient support apparatus has been completed.

The first function, in some aspects, is a zeroing of a scale system onboard the patient support apparatus.

The controller, in some aspects, is further adapted to automatically issue a second reminder to the user if the badge identifier belongs to the second class and the badge moves outside of the volume of space before a second function of the patient support apparatus has been completed, the second function being different from the first function.

The second function, in some aspects, is saving a weight of the patient.

The controller, in some aspects, is further adapted to monitor a plurality of conditions of the patient support apparatus, to determine if any of the conditions are not in a desired state, and to send a message to the server if any of the conditions are not in a desired state.

The server, in some aspects, is adapted to monitor a position of a plurality of badges and, in response to receiving the message, to automatically determine which badge of the plurality of badges is closest to the patient support apparatus and to send a second message to the badge closest to the patient support apparatus. The second message may indicate that at least one of the conditions is not in a desired state.

The server, in some aspects, is adapted to use the badge identifier to determine an identity of a user associated with the badge.

In some aspects, the server is adapted to determine a room identifier in which the patient support apparatus is positioned by using the locator identifier, and to use the room identifier to determine the patient identity.

The server, in some aspects, is further adapted to use the patient identity to determine the at least a portion of the care plan.

The server, in some aspects, is adapted to use the badge identifier to determine an identity of the user associated with the badge.

In some aspects, the server is further configured to transmit the identity of the user to a second server and to automatically log in the user to the second server. The second server may maintain an association between badge identifiers and user identities.

In some aspects, the selected badge is a badge currently positioned closest to a room in which the patient support apparatus is located.

In some aspects, the selected badge is a badge associated with a caregiver assigned to the patient supported by the patient support apparatus.

The server, in some aspects, is adapted to not send the second message to the selected badge if the badge is positioned inside the volume of space while at least one of the conditions is in an undesired state.

The controller, in some aspects, is adapted to send a third message directly to the badge if the badge is positioned inside the volume of space while at least one of the conditions is in an undesired state. The third message indicates that a condition of the patient support apparatus is in an undesired state.

In some aspects, the controller is adapted to use at least one of the plurality of ultra-wideband transceivers to send the third message directly to the badge.

The controller, in some aspects, is further adapted to detect when the first control is activated and the badge is positioned inside of the volume of space, to receive a badge identifier from the badge, and to transmit both the badge identifier and a function identifier to the server. The function identifier identifies the first function.

The server, in some aspects, is further adapted to perform at least one of the following: (i) record data indicating that the first function was implemented by the user with the badge identifier, or (ii) send a validation message to an electronic medical records server indicating that the user with the badge identifier implemented the first function.

According to another aspect of the present disclosure, a patient support apparatus is provided that includes a support surface adapted to support a patient, a plurality of ultra-wideband (UWB) transceivers, a network transceiver, and a controller. The network transceiver is adapted to communicate with a healthcare facility computer network. The controller is adapted to use radio frequency (RF) communication between the plurality of UWB transceivers and a badge to determine a position of the badge relative to the patient support apparatus. The controller is further adapted to receive a badge identifier from the badge; to determine if the badge is positioned inside or outside of a volume of space; to determine if a condition is in an undesired state; and, if the badge is positioned inside the volume of space and the condition is in the undesired state, to transmit a notification message to a person associated with the badge indicating that the condition is in the undesired state.

According to other aspects of the present disclosure, the controller is further adapted to not transmit the notification message to the person associated with the badge if the badge is positioned outside of the volume of space.

In some aspects, the undesired state is at least one of the following: a scale system onboard the patient support apparatus needing to be zeroed; or a power cord of the patient support apparatus not being plugged into an electrical outlet.

The controller, in some aspects, is adapted to transmit the notification message directly to the badge without passing through a server or a wireless access point.

In some aspects, the controller is adapted to transmit the notification message to a portable electronic device of the person associated with the badge.

The patient support apparatus, in some aspects, further includes a Bluetooth transceiver and the controller is adapted to transmit the notification message directly to the portable electronic device using the Bluetooth transceiver.

The controller, in some aspects, is adapted to transmit the notification message to the person associated with the badge using the network transceiver.

In some aspects, the patient support apparatus further includes a display and a control panel, and the controller is adapted to allow a user to access a first set of screens on the display if the badge identifier corresponds to a first class, and to allow the user to access a second set of screens on the display if the badge identifier corresponds to a second class. The first set of screens includes at least one screen not in the second set of screens.

The controller, in some aspects, is adapted to display at least a portion of a care plan for the patient if the badge identifier corresponds to the first class, and to not display any portion of the care plan for the patient if the badge identifier correspond to the second class.

In some aspects, the controller is further adapted to use RF communication between the plurality of UWB transceivers and a fixed locator to determine a position of the patient support apparatus relative to the fixed locator.

The controller, in some aspects, is further adapted to receive a locator identifier from the fixed locator, to determine if the patient support apparatus is positioned within a threshold distance of the fixed locator, and to forward the locator identifier to a server if the patient support apparatus is positioned within the threshold distance.

The patient support apparatus, in some aspects, further includes a microphone positioned onboard the patient support apparatus. The microphone is adapted to convert sounds of the patient's voice to audio signals, and the controller is further adapted to transmit the audio signals to the fixed locator if the fixed locator is positioned within the threshold distance, and to not transmit the audio signals to the fixed locator if the fixed locator is positioned outside of the threshold distance.

The undesired state, in some aspects, is at least one of the following: an amount of liquid in a bag used within an infusion pump is below a liquid threshold; an amount of oxygen in an oxygen bottle is below an oxygen threshold; the patient support apparatus needs to be marked as having been cleaned; one or more items in a closet or cabinet need to be restocked; equipment should be added to the patient support apparatus; equipment should be removed from the patient support apparatus; or the patient support apparatus should be plugged into a charging station.

In some aspects, the UWB transceivers are adapted to receive information from a transceiver associated with an infusion pump indicative of a level of liquid in a bag coupled to the infusion pump, and the notification message indicates that the level of the liquid in the bag is below a threshold.

The UWB transceivers, in some aspects, are adapted to receive information from a transceiver associated with an oxygen bottle indicative of a level of remaining oxygen in the oxygen bottle, and the notification message indicates that the level of the remaining oxygen in the oxygen bottle is below a threshold.

In some aspects, the UWB transceivers are adapted to communicate with a badge associated with a cleaning person, and the notification message indicates that the patient support apparatus should be marked as having been cleaned.

The UWB transceivers, in some aspects, are adapted to communicate with tagged items stored in a closet or cabinet, and the controller is adapted to determine whether one or more of the tagged items should be restocked. The notification message indicates that the one or more of the tagged items should be restocked.

The controller, in some aspects, is adapted to receive data from the healthcare facility computer network indicative of a needed piece of equipment for caring for the patient. The UWB transceivers are adapted to determine if the needed piece of equipment is within a threshold distance of the patient support apparatus, and the notification message indicates that the needed piece of equipment is not within the threshold distance of the patient support apparatus.

The controller, in some aspects, is adapted to receive data from the healthcare facility computer network indicative of one or more needed pieces of equipment for caring for the patient. The UWB transceivers are adapted to determine if a piece of equipment is within a threshold distance of the patient support apparatus and, if so, the controller is adapted to determine if the piece of equipment is a needed piece of equipment or an unneeded piece of equipment. The notification message indicates that the piece of equipment is an unneeded piece of equipment.

The UWB transceivers, in some aspects, are adapted to communicate with a charging station and the controller is adapted to determine both if the patient support apparatus is within a threshold distance of the charging station and a cable has been connected between the patient support apparatus and the charging station. The notification message indicates that the cable should be connected between the patient support apparatus and the charging station.

According to another aspect of the present disclosure, a software application embodied in a non-transitory computer readable medium is provided. The software application is adapted, when executed by a processor of a server, to instruct the server to perform the following: receive a message from a patient support apparatus indicating a condition is in an undesired state, receive a data indicating a proximity of a caregiver badge to the patient support apparatus, and send a notification message to a caregiver if the caregiver badge is less than a threshold distance from the patient support apparatus. The notification message indicates that the condition is in the undesired state.

According to other aspects of the present disclosure, the software application is adapted to instruct the server to receive the data indicating a proximity of the caregiver badge to the patient support apparatus from the patient support apparatus.

In some aspects, the software application is adapted to instruct the server to receive the data indicating a proximity of the caregiver badge to the patient support apparatus from a real-time locating system.

In some aspects, the software application is adapted to instruct the server to receive the data indicating a proximity of the caregiver badge to the patient support apparatus from the caregiver badge.

In some aspects, the software application is adapted to instruct the server to not transmit the notification message to the caregiver if the caregiver badge is positioned outside the threshold distance.

The software application, in some aspects, is adapted to instruct the server to transmit the notification message to the caregiver's badge.

The software application, in some aspects, is adapted to instruct the server to transmit the notification message to a smart phone associated with the caregiver.

The undesired state, in some aspects, is at least one of the following: a scale system onboard the patient support apparatus needs to be zeroed; a power cord of the patient support apparatus is not plugged into an electrical outlet; an amount of liquid in a bag used within an infusion pump is below a liquid threshold; an amount of oxygen in an oxygen bottle is below an oxygen threshold; the patient support apparatus needs to be marked as having been cleaned; one or more items in a closet or cabinet need to be restocked; equipment should be added to the patient support apparatus; equipment should be removed from the patient support apparatus; or the patient support apparatus should be plugged into a charging station.

The software application, in some aspects, is adapted to instruct the server to receive information from the patient support apparatus indicative of a level of liquid in a bag coupled to an infusion pump, and the notification message indicates that the level of the liquid in the bag is below a threshold.

In some aspects, the software application is adapted to instruct the server to receive information from the patient support apparatus indicative of a level of remaining oxygen in an oxygen bottle, and the notification message indicates that the level of the remaining oxygen in the oxygen bottle is below a threshold.

The software application, in some aspects, is adapted to instruct the server to communicate with a badge associated with a cleaning person, and the notification message indicates that the patient support apparatus should be marked as having been cleaned.

In some aspects, the software application is adapted to instruct the server to receive information from the patient support apparatus indicative of one or more of tagged items needing to be restocked, and the notification message indicates that the one or more of the tagged items should be restocked.

In some aspects, the software application is adapted to instruct the server to receive data from another server indicative of a needed piece of equipment for caring for the patient, to receive data from the patient support apparatus indicative of the absence of the needed piece of equipment within a vicinity of the patient support apparatus, and the notification message indicates that the needed piece of equipment is not within the vicinity of the patient support apparatus.

In some aspects, the software application is adapted to instruct the server to receive data from another server indicative of one or more needed pieces of equipment for caring for the patient, to receive data from the patient support apparatus indicative of a presence of a piece of equipment, to determine if the piece of equipment is a needed piece of equipment or an unneeded piece of equipment, and the notification message indicates that the piece of equipment is an unneeded piece of equipment.

The software application, in some aspects, is adapted to instruct the server to receive data from the patient support apparatus indicative of the patient support apparatus being within a threshold distance of a charging station and a cable not being been connected between the patient support apparatus and the charging station, and the notification message indicates that the cable should be connected between the patient support apparatus and the charging station.

According to another aspect of the present disclosure, a patient support apparatus system is provided that includes a patient support apparatus and a server. The patient support apparatus includes a support surface adapted to support a patient, a plurality of UWB transceivers, a network transceiver, and a controller. The network transceiver is adapted to communicate with the server via a healthcare facility computer network. The controller is adapted to use radio frequency (RF) communication between the plurality of UWB transceivers and a fixed locator to determine a location of the patient support apparatus relative to the fixed locator, to use radio frequency (RF) communication to determine a position one or more inventory items, to forward a first identifier received from the fixed locator to the server, and to forward a first number to the server indicating how many of the inventory items are within a first volume of space. The server is adapted to use the first identifier to determine a specific room within a healthcare facility in which the patient support apparatus is currently located, to compare the first number to a target number of inventory items, and to issue a notification if the first number is less than the target number.

The first volume of space, in some aspects, corresponds to at least one of a closet, a storage compartment, or a drawer.

In some aspects, the server is adapted to issue the notification by sending a notification message to a caregiver badge associated with a caregiver assigned to care for the patient in the patient support apparatus.

The server, in some aspects, is adapted to issue the notification by sending a notification message to a caregiver badge that is positioned within a threshold distance of the patient support apparatus.

In some aspects, the server is adapted to issue the notification by sending a notification message to the patient support apparatus.

The controller, in some aspects, is adapted to forward the notification message to a caregiver badge detected by the UWB transceivers to be within a threshold distance of the patient support apparatus.

In some aspects, the controller is further adapted to receive a second identifier from each of the inventory items, and to forward the second identifier to the server. The server is adapted to use the second identifier to determine a type of product the inventory item corresponds to.

the controller, in some aspects, is adapted to forward a second number to the server indicating how many of the inventory items are within a second volume of space different from the first volume of space, and the server is adapted to compare the second number to a second target number of inventory items, and to issue a second notification if the second number is less than the second target number.

In some aspects, the first volume of space corresponds to a first one a closet, a storage compartment, or a drawer, and the second volume of space corresponds to a second one of the closet, the storage compartment, or the drawer.

In some aspects, the controller is adapted to determine the first number by using the plurality of UWB transceivers to determine a three-dimensional position of each of the inventory items relative to the patient support apparatus, and to compare the three-dimensional position of each inventory item to the first volume of space.

The controller, in some aspects, is adapted to determine the first number by communicating with a UWB storage unit, wherein the UWB storage unit transmits the first number to the patient support apparatus.

In some aspects, the patient support apparatus further includes a power cord adapted to receive electrical power from an external source, and the controller is further adapted to use radio frequency (RF) communication between the plurality of UWB transceivers and a charging station to determine a position of the charging station relative to the patient support apparatus. The controller is further adapted to determine if the charging station is positioned within a threshold distance of the patient support apparatus; to determine if the power cord is electrically decoupled from the charging station; and, if the charging station is within the threshold distance and the power cord is decoupled from the charging station, to issue a second notification.

In some aspects, the controller is adapted to issue the second notification by displaying a message on a display of the patient support apparatus.

The controller, in some aspects, is adapted to issue the second notification by transmitting a second notification message to a caregiver badge.

According to another aspects of the present disclosure, a patient support apparatus is provided that includes a support surface adapted to support a patient, a plurality of UWB transceivers, a power cord, a network transceiver, and a controller. The power cord is adapted to receive electrical power from an external source. The network transceiver is adapted to communicate with a healthcare facility computer network. The controller is adapted to use radio frequency (RF) communication between the plurality of UWB transceivers and a charging station to determine a position of the charging station relative to the patient support apparatus. The controller is further adapted to determine if the charging station is positioned within a threshold distance of the patient support apparatus; to determine if the power cord is electrically decoupled from the charging station; and, if charging station is within the threshold distance and the power cord is decoupled from the charging station, to issue a notification.

In some aspects, the controller is adapted to issue the notification by displaying a message on a display of the patient support apparatus.

The controller, in some aspects, is adapted to issue the notification by transmitting a notification message to a caregiver badge.

In some aspects, the controller is adapted to issue the notification by transmitting a notification message to the charging station.

The controller, in some aspects, is adapted to use radio frequency (RF) communication between the plurality of UWB transceivers and a caregiver badge to determine a position of the caregiver badge relative to the patient support apparatus, to determine if the caregiver badge is positioned inside or outside of a first volume of space, and to issue the notification by transmitting a notification message to the caregiver badge if the caregiver badge is inside the first volume of space.

The controller, in some aspects, is adapted to determine if the caregiver badge is positioned inside or outside of a second volume of space smaller than the first volume of space, and to cancel the notification if the caregiver badge moves from inside the first volume of space to inside the second volume of space.

In some aspects, the controller is adapted to use radio frequency (RF) communication between the plurality of UWB transceivers and a fixed locator to determine a location of the patient support apparatus relative to the fixed locator, to use radio frequency (RF) communication to determine a position one or more inventory items, to forward a first identifier received from the fixed locator to a server, and to forward a first number to the server indicating how many of the inventory items are within a first volume of space.

In some aspects, the controller is further adapted to receive a restocking message from the server, and to issue a restocking notification in response to receiving the restocking message.

The controller, in some aspects, is adapted to issue the restocking notification by displaying a restocking message on a display of the patient support apparatus.

In some aspects, the controller is adapted to issue the restocking notification by transmitting a restocking message to a caregiver badge.

The controller, in some aspects, is adapted to issue the restocking notification by transmitting a restocking message to a restocking unit.

The controller, in some aspects, is adapted to use radio frequency (RF) communication between the plurality of UWB transceivers and a badge to determine a position of the badge relative to the patient support apparatus, to determine if the badge is positioned inside or outside of a volume of space, to determine if a condition is in an undesired state, and, if the badge is positioned inside the volume of space and the condition is in the undesired state, to transmit a notification message to a person associated with the badge indicating that the condition is in the undesired state.

In some aspects, the controller is further adapted to not transmit the notification message to the person associated with the badge if the badge is positioned outside of the volume of space.

According to another aspect of the present disclosure, a patient support apparatus is provided that includes a support surface adapted to support a patient, a plurality of UWB transceivers, a battery, a network transceiver, and a controller. The network transceiver is adapted to communicate with a healthcare facility computer network. The controller is adapted to use radio frequency (RF) communication between the plurality of UWB transceivers and a badge to determine a position of the badge relative to the patient support apparatus, to receive a badge identifier from the badge, to determine if the badge is positioned inside or outside of a volume of space, to determine a charge level of the battery, and, if the badge is positioned inside the volume of space and the charge level of the battery is above a first threshold, to transmit a battery status message to a person associated with the badge indicating that the battery is at a charge level above the first threshold.

The controller, in some aspects, is further adapted to not transmit the battery status message to the person associated with the badge if the badge is positioned outside of the volume of space.

In some aspects, the controller is adapted to transmit the battery status message directly to the badge without passing through a server or a wireless access point.

The controller, in some aspects, is adapted to transmit the battery status message to a portable electronic device of the person associated with the badge.

The patient support apparatus, in some aspects, further includes a Bluetooth transceiver, and the controller is adapted to transmit the battery status message directly to the portable electronic device using the Bluetooth transceiver.

The controller, in some aspects, is adapted to transmit the battery status message to the person associated with the badge using the network transceiver.

According to another aspect of the present disclosure, a patient support apparatus is provided that includes a support surface adapted to support a patient, a plurality of UWB transceivers, an emergency button, a network transceiver, and a controller. The network transceiver is adapted to communicate with a healthcare facility computer network. The controller is adapted to use radio frequency (RF) communication between the plurality of UWB transceivers and a fixed locator to determine a location of the patient support apparatus relative to the fixed locator. The controller is further adapted to detect when the emergency button is pressed and to use at least one of the plurality of UWB transceivers to forward an emergency alert to a caregiver badge. The emergency button alert includes an identifier of a room in which the patient support apparatus is currently located.

Before the various aspects of the disclosure are explained in detail, it is to be understood that the claims are not to be limited to the details of operation or to the details of construction and the arrangement of the components set forth in the following description or illustrated in the drawings. The aspects described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to a first aspect of the present disclosure;

FIG. 2 is a plan view of an illustrative caregiver control panel of the patient support apparatus of FIG. 1;

FIG. 3 is a plan view of an illustrative patient control panel of the patient support apparatus of FIG. 1;

FIG. 4 is a perspective view of a patient support apparatus system of the present disclosure showing the patient support apparatus, a device, a display device, a caregiver with a badge, and a linked locator unit that is used for automatically detecting the location of a patient support apparatus;

FIG. 5 is a block diagram of the patient support apparatus system of FIG. 4;

FIG. 6 is a plan view of an illustrative healthcare facility area, a patient support apparatus, a plurality of caregiver badges, a device, a plurality of space volumes, and a plurality of wall units;

FIG. 7 is a block diagram of an unlinked locator unit, the healthcare facility network, and a caregiver badge;

FIG. 8 is a diagram of the system of patient support apparatus system of FIG. 4 showing more details of some of the data contained in a plurality of servers that may be resident in the healthcare facility;

FIG. 9 is an example of a screen that may be displayed on a remote electronic device that shows the status of multiple patient support apparatuses, medical devices, and activities performed by caregivers, and other information;

FIG. 10 is an illustrative example of a menu screen that may be displayed on the caregiver control panel of the patient support apparatus for a first class of users;

FIG. 11 is an illustrative example of a menu screen that may be displayed on the caregiver control panel of the patient support apparatus for a second class of users; and

FIG. 12 is a perspective view of the patient support apparatus shown positioned next to a charging station.

### DETAILED DESCRIPTION OF THE EMBODIMENTS

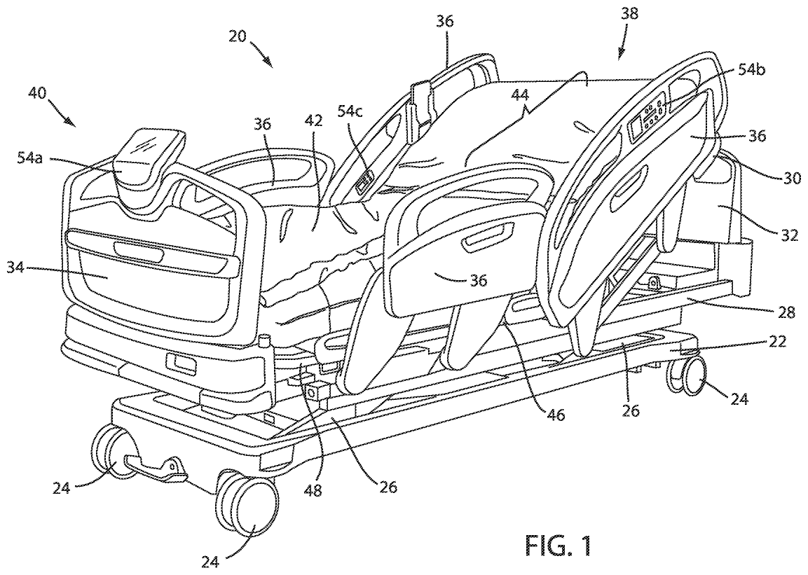

An illustrative patient support apparatus 20 according to an embodiment of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, or any other structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a headboard 32, a footboard 34 and a plurality of siderails 36. Siderails 36 are all shown in a raised position in FIG. 1 but are each individually movable to a lower position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. In some embodiments, the mattress 42 includes one or more inflatable bladders that are controllable via a blower, or other source of pressurized air. In at least one embodiment, the inflation of the bladders of the mattress 42 is controllable via electronics built into patient support apparatus 20. In one such embodiments, mattress 42 may take on any of the functions and/or structures of any of the mattresses disclosed in commonly assigned U.S. Pat. No. 9,468,307 issued Oct. 18, 2016, to inventors Patrick Lafleche et al., the complete disclosure of which is incorporated herein by reference. Still other types of mattresses may be used.

Support deck 30 is made of a plurality of sections, some of which are pivotable about generally horizontal pivot axes. In the embodiment shown in FIG. 1, support deck 30 includes at least a head section 44, a thigh section 46, and a foot section 48, all of which are positioned underneath mattress 42 and which generally form flat surfaces for supporting mattress 42. Head section 44, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width and/or length of patient support deck 30, thereby allowing patient support apparatus 20 to accommodate patients of varying sizes. When so modified, the width of deck 30 may be adjusted sideways and/or lengthwise in increments or otherwise.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions that are different from what is shown in the attached drawings, such as, but not limited to, the construction described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may include the same, or nearly the same, structures as the Model 3002 S3 bed manufactured and sold by Stryker Corporation of Kalamazoo, Michigan. This construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. In still another embodiment, the mechanical construction of patient support apparatus 20 may include the same, or nearly the same, structure as the Model 3009 Procuity MedSurg bed manufactured and sold by Stryker Corporation of Kalamazoo, Michigan. This construction is described in greater detail in the Stryker Maintenance Manual for the 3009 Procuity MedSurg bed (publication 3009-009-002, Rev. A.0), published in 2020 by Stryker Corporation of Kalamazoo, Michigan.

It will be understood by those skilled in the art that patient support apparatus 20 can be designed with still other types of mechanical constructions, such as, but not limited to, those described in commonly assigned, U.S. Pat. No. 7,690,59 issued Apr. 6, 2010, to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The overall mechanical construction of patient support apparatus 20 may also take on still other forms different from what is disclosed in the aforementioned references provided the patient support apparatus includes one or more of the functions, features, and/or structures discussed in greater detail below.

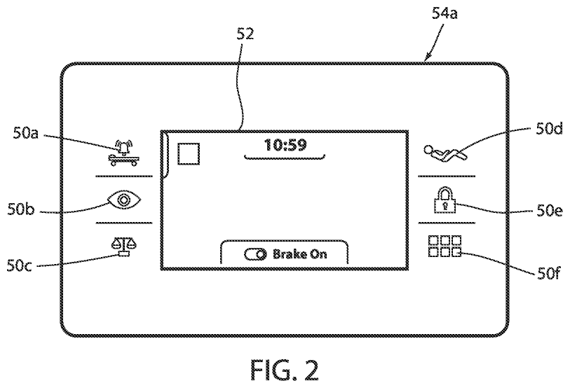

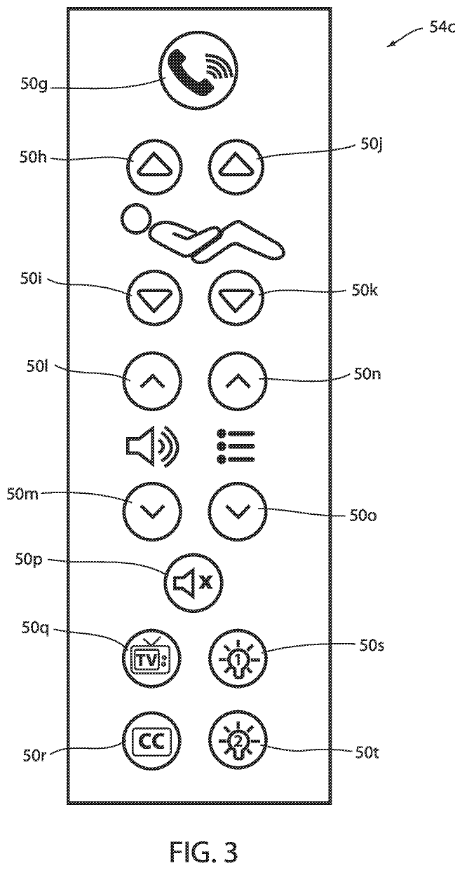

Patient support apparatus 20 further includes a plurality of control panels 54 that enable a user of patient support apparatus 20, such as a patient and/or an associated caregiver, to control one or more aspects of patient support apparatus 20. In the embodiment shown in FIG. 1, patient support apparatus 20 includes a footboard control panel 54a, a pair of outer siderail control panels 54b (only one of which is visible), and a pair of inner siderail control panels 54c (only one of which is visible). Footboard control panel 54a and outer siderail control panels 54b are intended to be used by caregivers, or other authorized personnel, while inner siderail control panels 54c are intended to be used by the patient associated with patient support apparatus 20. Each of the control panels 54 includes a plurality of controls 50 (see, e.g. FIGS. 2-3), although each control panel 54 does not necessarily include the same controls and/or functionality.

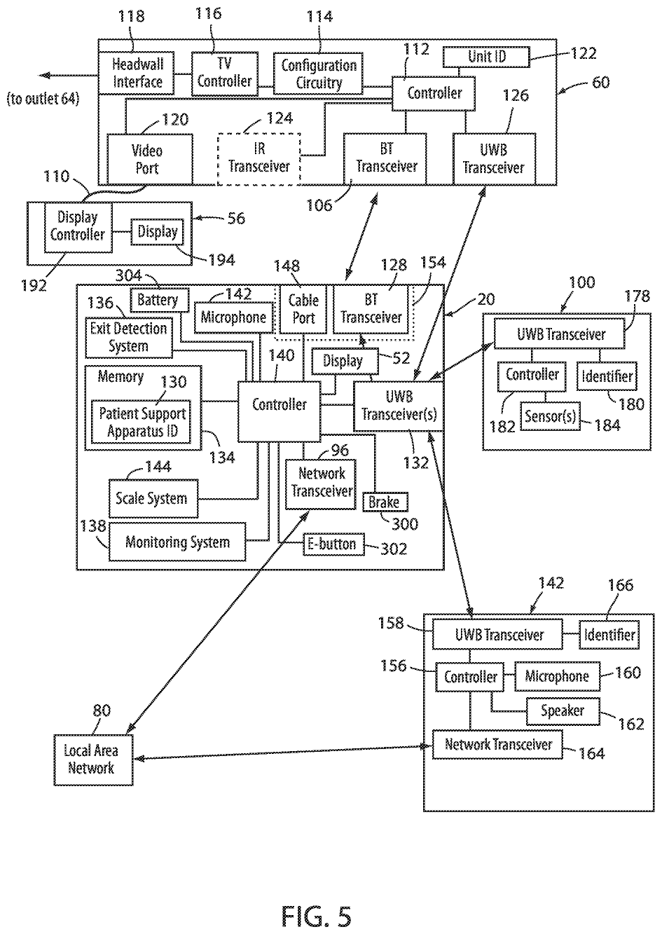

Among other functions, controls 50 of control panel 54a allow a user to control one or more of the following: change a height of support deck 30; raise or lower head section 44; activate and deactivate a brake 300 for wheels 24; arm and disarm an exit detection system 136 and/or an onboard monitoring system 138 (FIG. 5); change various settings on patient support apparatus 20; view the current location of the patient support apparatus 20 as determined by the location detection system discussed herein; view what devices, such as, but not limited to, medical devices, exercise devices, nurse call devices, caregiver badges, etc. that the patient support apparatus 20 has associated itself with; and perform other actions. One or both of the inner siderail control panels 54c also include at least one control that enables a patient to call a remotely located nurse (or other caregiver). In addition to the nurse call control, one or both of the inner siderail control panels 54c also include one or more controls for controlling one or more features of one or more room devices positioned within the same room as the patient support apparatus 20. As will be described in more detail below, such room devices include, but are not necessarily limited to, a television, a reading light, and a room light. With respect to the television, the features that may be controllable by one or more controls 50 on control panel 54c include, but are not limited to, the volume, the channel, the closed-captioning, and/or the power state of the television. With respect to the room and/or night lights, the features that may be controlled by one or more controls 50 on control panel 54c include the on/off state and/or the brightness level of these lights.

Control panel 54a includes a display 52 (FIG. 2) configured to display a plurality of different screens thereon. Surrounding display 52 are a plurality of navigation controls 50a-f that, when activated, cause the display 52 to display different screens on display 52. More specifically, when a user presses navigation control 50a, control panel 54a displays an exit detection control screen on display 52 that includes one or more icons that, when touched, control an onboard exit detection system 136 (FIG. 5). The exit detection system 136 is as adapted to issue an alert when a patient exits from patient support apparatus 20. Exit detection system 136 may include any of the same features and functions as, and/or may be constructed in any of the same manners as, the exit detection system disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. Other types of exit detection systems may be included within patient support apparatus 20.

When a user presses navigation control 50b (FIG. 2), control panel 54 displays a monitoring control screen that includes a plurality of control icons that, when touched, control the onboard monitoring system 138 (FIG. 5) built into patient support apparatus 20. The onboard monitoring system 138 alerts the caregiver through a unified indicator, such as a light or a plurality of lights controlled in a unified manner, when any one or more of a plurality of settings on patient support apparatus 20 are in an undesired state, and uses that same unified indicator to indicate when all of the plurality of settings are in their respective desired states. Stated alternatively, monitoring system 138, when armed, monitors a plurality of conditions of patient support apparatus 20 (such as, but not limited to, any one or more of the following: brake status, siderail position, litter frame height, exit detection system 136, A/C cord status, nurse call cable status, etc.) and issues an alert if any one of those conditions are in an undesired state. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20.

When a user presses navigation control 50c, control panel 54a displays a scale control screen that includes a plurality of control icons that, when touched, control a scale system 144 (FIG. 5) of patient support apparatus 20. Such a scale system 144 may include any of the same features and functions as, and/or may be constructed in any of the same manners as, the scale systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. The scale system 144 may utilize the same force sensors and/or other components that are utilized by the exit detection system 136, or it may utilize one or more different sensors and/or other components. In some embodiments, the scale system 144 and exit detection system 136 are combined into a single system. Other scale systems besides those mentioned above in the '254 and '954 applications may alternatively be included within patient support apparatus 20.

When a user presses navigation control 50d, control panel 54 displays a motion control screen that includes a plurality of control icons that, when touched, control the movement of various components of patient support apparatus 20, such as, but not limited to, the height of litter frame 28 and the pivoting of head section 44. In some embodiments, the motion control screen displayed on display 52 in response to pressing control 50d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be included on patient support apparatus 20.

When a user presses navigation control 50e, control panel 54a displays a motion lock control screen that includes a plurality of control icons that, when touched, control one or more motion lockout functions of patient support apparatus 20. Such motion lockout functions typically include the ability for a caregiver to use control panel 54a to lock out one or more of the motion controls 50 of the patient control panels 54c such that the patient is not able to use those controls 50 on control panels 54c to control the movement of one or more components of patient support apparatus 20. The motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

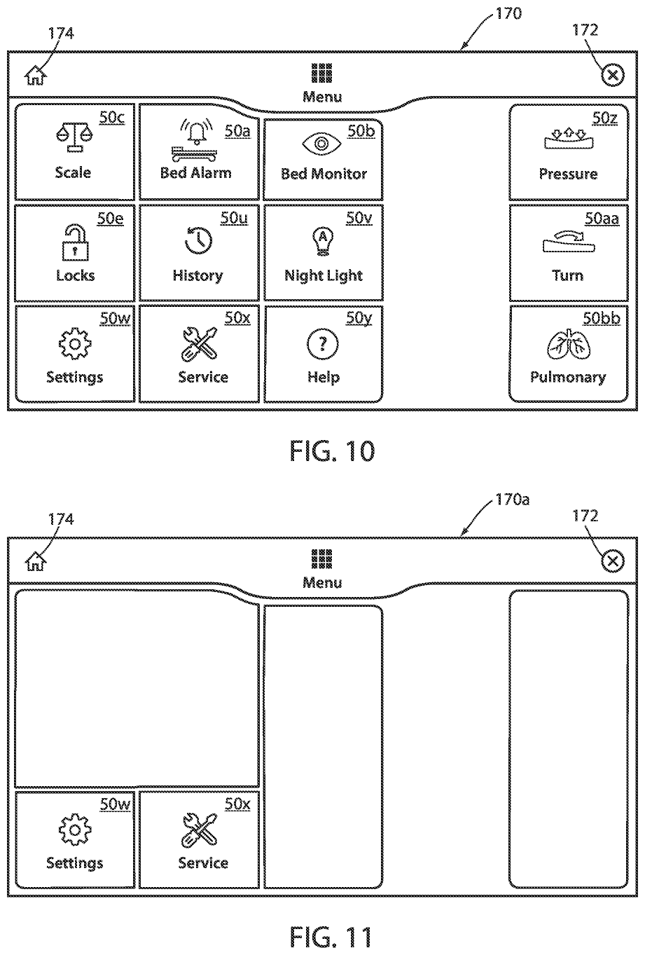

When a user presses on navigation control 50f, control panel 54a displays a menu screen that includes a plurality of menu icons that, when touched, bring up one or more additional screens for controlling and/or viewing one or more other aspects of patient support apparatus 20. Such other aspects include, but are not limited to, displaying information about one or more devices that are currently associated with patient support apparatus 20, diagnostic and/or service information for patient support apparatus 20, mattress control and/or status information, configuration settings, location information, and other settings and/or information. Examples of menu screens are shown in FIGS. 10 and 11, as well as the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20. In at least one embodiment, utilization of navigation control 50f allows a user to navigate to a screen that enables a user to see which devices, if any, are currently associated with patient support apparatus 20. As will be discussed in greater detail below, patient support apparatus 20 includes an onboard locating system that is adapted to automatically determine the relative position of one or more devices with respect to patient support apparatus 20 and, in some instances, automatically associate and/or disassociate those devices with and/or from patient support apparatus 20 (and/or the patient assigned to patient support apparatus 20) depending upon the proximity of the device to patient support apparatus 20. Further details of this locating system are provided below.

For all of the navigation controls 50a-f (FIG. 2), screens other than the ones specifically mentioned above may be displayed on display 52 in other embodiments of patient support apparatus 20 in response to a user pressing these controls. Thus, it will be understood that the specific screens mentioned above are merely representative of the types of screens that are displayable on display 52 in response to a user pressing on one or more of navigation controls 50a-f. It will also be understood that, although navigation controls 50a-f have all been illustrated in the accompanying drawings as dedicated controls that are positioned adjacent display 52, any one or more of these controls 50a-f could alternatively be touchscreen controls that are displayed at one or more locations on display 52. Still further, although controls 50a-f have been shown herein as buttons, it will be understood that any of controls 50a-f could also, or alternatively, be switches, dials, or other types of non-button controls. Additionally, patient support apparatus 20 may be modified to include additional, fewer, and/or different navigation controls from the navigation controls 50a-f shown in FIG. 2.

FIG. 3 illustrates one example of a patient control panel 54c that may be incorporated into patient support apparatus 20 and positioned at a location on patient support apparatus 20 that is convenient for a patient to access while supported on support deck 30, such as on an interior side of one of the siderails 36. Control panel 54c includes a plurality of controls 50g-t that are intended to be operated by a patient. A nurse call control 50g, when pressed by the patient, sends a signal to a nurse call system requesting that a remotely positioned nurse talk to the patient. A Fowler-up control 50h, when pressed by the patient, causes a motorized actuator onboard patient support apparatus 20 to raise Fowler section 44 upwardly. A Fowler-down control 50i, when pressed by the patient, causes the motorized actuator to lower Fowler section 44 downwardly. A gatch-up control 50j, when pressed by the patient, causes another motorized actuator to raise a knee section of support deck 30, while a gatch-down control 50k causes the motorized actuator to lower the knee section of support deck 30. The knee section may refer to the joint that couples thigh section 46 to foot section 48.

A volume-up control 50l, when pressed by the patient, causes patient support apparatus 20 to send a signal to an in-room television instructing it to increase its volume, while a volume down control 50m, when pressed, causes patient support apparatus 20 to send a signal to the television instructing it to decrease its volume. A channel-up control 50n, when pressed by the patient, causes patient support apparatus 20 to send a signal to the television instructing it to increase the channel number, while a channel-down control 50o, when pressed, causes patient support apparatus 20 to send a signal to the television instructing it to decrease the channel number.

A mute control 50p, when pressed, causes patient support apparatus 20 to send a signal to the television instructing it to either mute itself or unmute itself, depending upon whether the television is currently muted or unmuted. In other words, mute control 50p is a toggle control that alternatingly sends mute and unmute commands to the television when it is pressed.

Power control 50q is a toggle control that, when pressed, sends a signal to the television to either turn on or turn off, depending upon the television's current power status. Closed-captioning control 50r is another toggle control that, when pressed, sends a signal to the television to either turn on its closed-captioning feature or to turn off its closed captioning feature, depending upon whether the closed-captioning feature is currently on or off.

Control 50s is a toggle control that, when pressed, sends a signal to a first light to either turn on or turn off, depending upon the current state of that first light. Control 50t is another toggle control that, when pressed, sends a signal to a second light to either turn on or turn off, depending upon the current state of that second light. In some embodiments, the first light is a reading light and the second light is a room light, both of which are positioned off-board the patient support apparatus 20.

It will be understood that not only the number of controls 50 on control panel 54c, but also the functions of the controls 50 on control panel 54c, the layout of the controls 50 on control panel 54c, and/or other aspects of control panel 54c may be modified from what is shown in FIG. 3. In some embodiments, control panel 54c is implemented on a pendant controller that includes a cable that is plugged into a port on patient support apparatus 20. In other embodiments, one or more of the controls 50 of control panel 54c may be omitted, augmented, and/or split amongst other controls panels and/or locations. Still other manners of implementing control panel 54c are also possible.

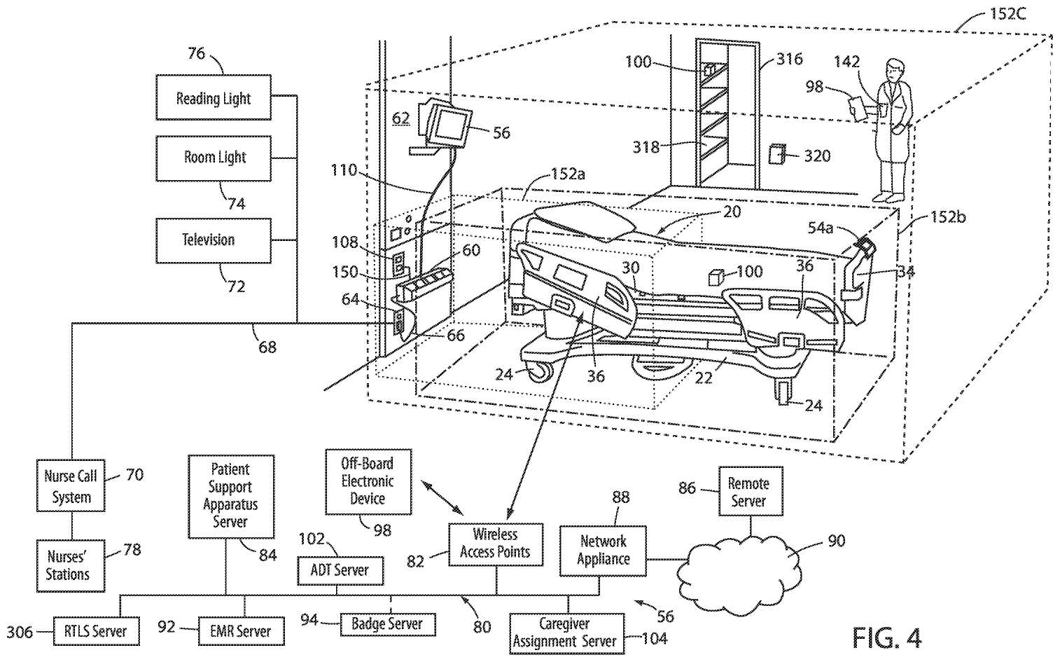

FIG. 4 illustrates patient support apparatus 20 positioned within a room 58 of a healthcare facility. FIG. 4 also illustrates additional items that may be present in a healthcare facility and which patient support apparatus 20 is configured to communicate with, including, but not limited to, a locator unit 60, a conventional local area network 80 of the healthcare facility, a caregiver badge 142, and one or more other devices 100 used during the care of a patient. Locator units 60 are positioned at known and fixed locations within the healthcare facility in which patient support apparatus 20 is positioned. Locator units 60 function as fixed locators. That is, locator units 60 communicate with patient support apparatuses 20 and share information with them that allows the location of the patient support apparatuses 20 to be determined.

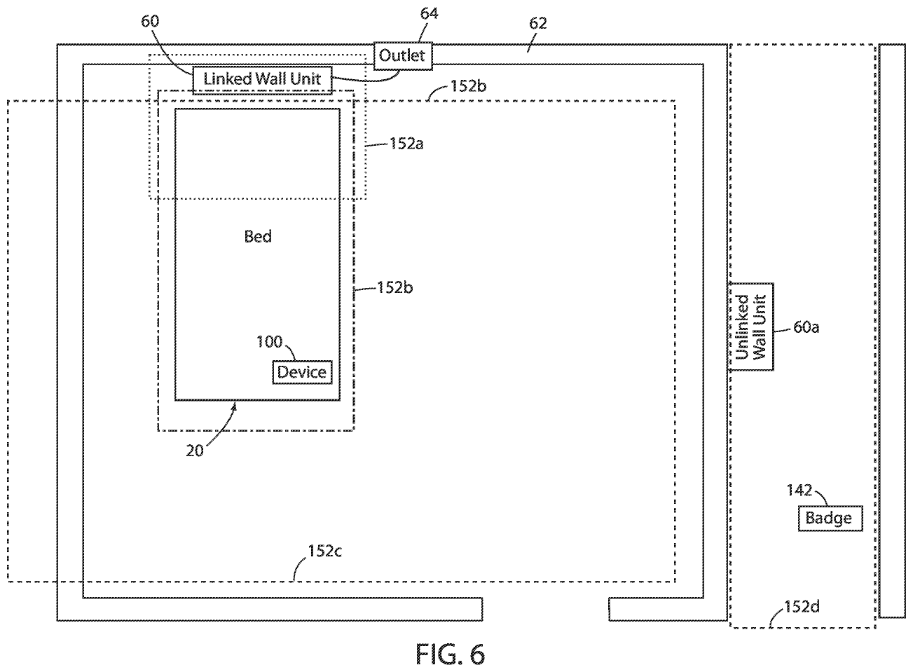

In some embodiments, patient support apparatus 20 is configured to be able to communicate with at least two different types of locator units 60: linked locator units 60 and unlinked locator units 60a. One example of a linked locator unit 60 is shown in FIG. 4. One example of an unlinked locator unit 60a is shown in FIG. 6. Other examples of unlinked locator units 60 are shown (and referred to as unlinked locator units 60b) in commonly assigned U.S. patent application Ser. No. 63/306,279 filed Feb. 3, 2022, by inventors Madhu Sandeep Thota et al. and entitled COMMUNICATION SYSTEM FOR PATIENT SUPPORT APPARATUSES, the complete disclosure of which is incorporated herein by reference. Patient support apparatus 20 is configured to communicate with the unlinked locator units 60b described in the aforementioned '279 application and to perform any one or more of the functions described therein that utilize such unlinked locator units and/or information provided by such unlinked locator units. Unless explicitly stated otherwise, all references herein to “locator units 60” without the term “linked” or “unlinked” in the reference will refer to both linked and unlinked locator units 60.

Linked locator units 60 are communicatively linked to a conventional communication outlet 64 and are adapted to provide location information to patient support apparatus 20. Linked locator units 60 are also adapted to serve as a communication conduit for routing wireless communications between patient support apparatus 20 and one or more devices and/or systems that are communicatively coupled to communication outlet 64 (e.g. room devices 72, 74, 76, and/or nurse call system 70, FIG. 4). In general, linked locator units 60 are typically positioned in patient rooms of the healthcare facility where one or more communication outlets 64 are typically present.

As shown in FIG. 4, linked locator unit 60 is adapted to be mounted to a wall 62, such as a headwall of a patient room 58 within the healthcare facility. The headwall of a conventional healthcare facility room 58 typically includes a conventional communications outlet 64 physically integrated therein. Communications outlet 64 is adapted to receive a nurse call cable 66 that physically connects at its other end either to patient support apparatus 20 (not shown) or to linked locator unit 60 (shown in FIG. 4). In many healthcare facilities, communication outlet 64 includes a 37-pin connector, although other types of connectors are often found in certain healthcare facilities. As will be discussed in greater detail below, linked locator unit 60 and nurse call cable 66 allow patient support apparatus 20 to communicate with a nurse call system, and one or more room devices positioned within room 58.

Communication outlet 64 is electrically coupled to one or more cables, wires, or other conductors 68 that electrically couple the communication outlet 64 to a nurse call system 70 and one or more conventional room devices, such as a television 72, a room light 74, and/or a reading light 76. Conductors 68 are typically located behind wall 62 and not visible. In some healthcare facilities, conductors 68 may first couple to a room interface circuit board that includes one or more conductors 68 for electrically coupling the room interface circuit board to room device 72, 74, 76 and/or nurse call system 70. Still other communicative arrangements for coupling communication outlet 64 to nurse call system 70 and/or one or more room devices 72, 74, 76 are possible.

Nurse call cable 66 (FIG. 4) enables linked locator unit 60 to communicate with nurse call system 70 and/or room devices 72, 74, 76. Because patient support apparatus 20 is able to wirelessly communicate with linked locator unit 60, patient support apparatus 20 is thereby able to communicate with nurse call system 70 and room devices 72, 74, 76. A patient supported on patient support apparatus 20 who activates a nurse call control (e.g. 50g; see FIG. 3) on patient support apparatus 20 causes a signal to be wirelessly sent from patient support apparatus 20 to linked locator unit 60, which in turn conveys the signal via nurse call cable 66 to the nurse call system 70, which forwards the signal to one or more remotely located nurses (e.g. nurses at one or more nurse's stations 78). If the patient activates one or more room device controls (e.g. controls 50l-t; see FIG. 3), one or more wireless signals are conveyed to linked locator unit 60, which in turn sends appropriate signals via nurse call cable 66 to communication outlet 64 and the room device 72, 74, 76 that change one or more features of these devices (e.g. the volume, channel, on/off state, etc.).

As is also shown in FIG. 4, patient support apparatus 20 is further configured to communicate with a local area network 80 of the healthcare facility. In the embodiment shown in FIG. 4, patient support apparatus 20 includes a wireless network transceiver 96 (FIG. 5) that communicates wirelessly with local area network 80. Network transceiver 96 is, in at least some embodiments, a WiFi transceiver (e.g. IEEE 802.11) that wirelessly communicates with one or more conventional wireless access points 82 of local area network 80. In other embodiments, network transceiver 96 may be a wireless transceiver that uses conventional 5G technology to communicate with network 80, one or more servers hosted thereon, and/or other devices. In some embodiments, network transceiver 96 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In some embodiments, network transceiver 96 is a wired transceiver that is adapted to allow patient support apparatus 20 to communicate with network 80 via a wired connection, such as an Ethernet cable that plugs into an Ethernet port (e.g. an RJ-45 style port, an 8P8C port, etc.) built into patient support apparatus 20. In still other embodiments, patient support apparatus 20 includes both a wired transceiver 96 for communicating with network 80 via a wired connection and a wireless transceiver 96 for wirelessly communicating with network 80.

Patient support apparatus 20 is configured to communicate with one or more servers on local area network 80 of the healthcare facility. One such server is a patient support apparatus server 84. Patient support apparatus server 84 is adapted, in at least one embodiment, to execute a software application that carries out any one or more of the functions described herein. In general, the software application instructs server 84 to receive data from the patient support apparatuses 20 positioned within the healthcare facility and distribute this data to caregivers, other servers, and/or other software applications. As will be discussed in greater detail below, server 84 may also be configured to receive data from one or more devices 100 and/or badges 142 that are positioned within one or more volumes of space defined around patient support apparatus 20 and/or within a volume of space defined around locator units 60. Such information may be forwarded from the device(s) to patient support apparatus 20, and from patient support apparatus 20 to server 84 via network transceiver 96 and one or more access points 82. In some embodiments where data from medical devices is collected, the data from one or more of the medical devices may be forwarded to an Electronic Medical Records (EMR) server 92, and/or to one or more other servers on network 80 (and/or one or more electronic devices 98), such as a caregiver assistance server and/or a caregiver assistance software application, as will also be discussed in greater detail below.

In some embodiments, patient support apparatus server 84 is configured to communicate at least some of the patient support apparatus data and/or device data received from patient support apparatuses 20 to a remote server 86 that is positioned geographically remotely from the healthcare facility. Such communication may take place via a conventional network appliance 88, such as, but not limited to, a router and/or a gateway, that is coupled to the Internet 90. The remote server 86, in turn, is also coupled to the Internet 90, and patient support apparatus server 84 is provided with the URL and/or other information necessary to communicate with remote server 86 via the Internet connection between network 80 and server 86.

In some alternative embodiments, patient support apparatus 20 may be configured to communicate directly with one or more cloud-based servers, such as remote server 86, without utilizing patient support apparatus server 84. That is, in some embodiments, patient support apparatuses 20 may be configured to communicate directly with a remote server without relying upon any locally hosted servers (e.g. servers hosted on network 80). Patient support apparatus 20 is provided with the URL and/or other information necessary to communicate with remote server 86 via the Internet connection between network 80 and remote server 86. In one embodiment, patient support apparatus 20 utilizes Microsoft's Azure could computing service to directly connect to one or more remote servers 86 without utilizing server 84. In some such embodiments, network appliance 88 is a router configured to support such direct connections. Still other types of direct-to-cloud connections may be utilized with one or more of patient support apparatuses 20. When patient support apparatus 20 is configured to directly communicate with remote server 86, patient support apparatus server 84 may be omitted and any one or more of the functions of patient support apparatus server 84 described herein may be performed by remote server 86.

Patient support apparatus server 84 is also configured to determine the location of each patient support apparatus 20 (and/or its associated devices), or receive the location of each patient support apparatus 20 (and/or its associated devices) from the patient support apparatuses 20. In some embodiments, patient support apparatus server 84 determines the room number and/or bay area of each patient support apparatus 20 and its associated devices that are positioned within a room 58, as well as the location of patient support apparatuses 20 and their associated devices that are positioned outside of a room 58, such as those that may be positioned in a hallway, a maintenance area, or some other area. In general, patient support apparatus server 84 may be configured to determine the position of any patient support apparatus 20 that is positioned within communication range of one or more locator units 60, as well as the location of any associated devices that are positioned within one or more volumes of space defined around the patient support apparatus 20, as will be discussed in greater detail below.

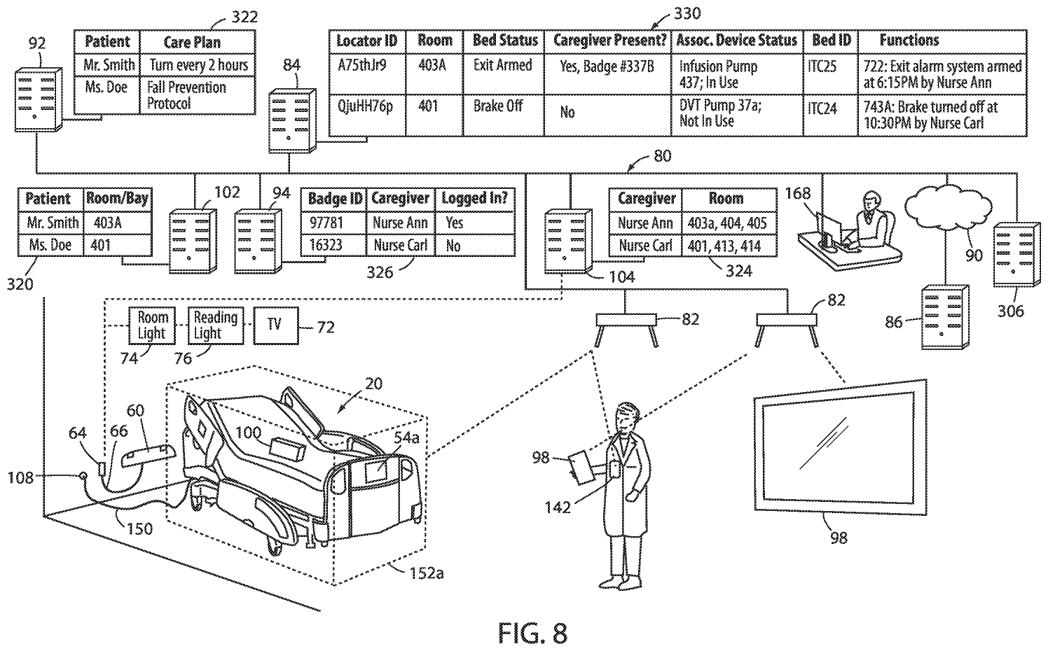

Patient support apparatus server 84 (FIGS. 4 & 8) is adapted to communicate with a plurality of other servers, such as a conventional EMR server 92, a conventional badge server 94, a conventional Admission, Discharge, and Transfer (ADT) server 102, a conventional Real Time Locating Service (RTLS) server 306, and/or a conventional caregiver assignment server 104. EMR server 92 stores individual patient records. Such patient records identify a patient by name and include medical information associated with that patient. Such medical information may include all of the medical information generated from the patient's current stay in the healthcare facility as well as medical information from previous visits. EMR server 92 stores data such as that shown in table 322 (FIG. 8). Table 322 shows an abbreviated example of several types of medical information entries that are commonly found within a patient's medical records: a fall risk entry indicating whether the patient is a fall risk, a bed sore risk entry indicating whether the patient is at risk for developing bed sores, and a care plan for a particular patient. EMR server 92 includes far more additional information in the medical records of each patient than what is shown in table 322. It will be understood that the term “EMR server,” as used herein, also includes Electronic Health Records servers, or EHR servers for short, and that the present disclosure does not distinguish between electronic medical records and electronic health records.

Caregiver assignment server 104 (FIGS. 4 and 8) includes a caregiver assignment table 324 that matches caregivers to specific rooms and/or bays within the healthcare facility. Caregiver assignment server 104 stores information regarding shift changes, personnel, and the general assignments of caregivers who are employed by the healthcare facility. Although table 324 only shows two caregivers who are each assigned to three rooms, it will be understood that more than two caregivers will typically be present in a healthcare facility and they may have lessor or greater numbers of room assignments. In some caregiver assignment servers 104, caregivers are assigned to specific patients, rather than to specific rooms, in which case table 324 may correlate caregivers to individual patients rather than rooms. Still further, some conventional nurse call systems may be configured to carry out the functions of caregiver assignment server 104, in which case caregiver assignment server 104 may be replaced by and/or supplemented with a nurse call server. FIG. 8 shows an example of this where caregiver assignment server 104 is coupled by a dotted line to communication outlet 64. In this particular example, caregiver assignment server 104 also functions as a nurse call server that oversees the communications between patients and their caregivers.

ADT server 102 stores patient information, including the identity of patients and the corresponding rooms 58 and/or bays within rooms to which the patients are assigned. That is, ADT server 102 includes a patient-room assignment table 320 (FIG. 8). The patient-room assignment table 320 correlates rooms, as well as bays within multi-patient rooms, to the names of individual patients within the healthcare facility. The patient's names are entered into the ADT server 102 by one or more healthcare facility staff whenever a patient checks into the healthcare facility and the patient is assigned to a particular room within the healthcare facility. If and/or when a patient is transferred to a different room and/or discharged from the healthcare facility, the staff of the healthcare facility update ADT server 102. ADT server 102 therefore maintains an up-to-date table 320 that correlates patient names with their assigned rooms and/or bays. In some conventional electronic medical record systems, the functions of the ADT server 102 may be incorporated into the EMR system, and EMR server 92 may therefore, in some embodiments, carry out the functions of ADT server 102.

RTLS server 306 (FIGS. 4 & 8) determines the locations of assets and individuals within a particular healthcare facility using one or more conventional locating technologies that are separate from, and independent of, the ultra-wideband locating technology implemented on patient support apparatuses 20. In some embodiments, RTLS server 306 determines the location of people and/or equipment by monitoring which wireless access points 82 within a healthcare facility are used by the people and/or equipment. Based on the known location of the access points 82, the location of the people and/or equipment can be determined. Alternatively, or additionally, RTLS server 306 may determine the location of people and/or equipment using Radio Frequency identification (RFID) tags that are worn by individuals and/or coupled to equipment. Fixed RFID transceivers may be positioned within the healthcare facility at known location and the location of the RFID tags can be determined by their communication with these fixed RFID transceivers using conventional triangulation, trilateration, and/or other means. Still other types of RTLS system may also or alternatively be used in some environments in which patient support apparatuses 20 are used.

Badge server 94 (FIGS. 4 & 8) is configured to manage communications between, and keep track of the location of, badges 142 that are typically worn by healthcare workers, such as caregivers, service technicians, cleaning personnel, transportation assistants, etc. Badge server 94 typically maintains a table 326 (FIG. 8) that correlates badge IDs with individual healthcare workers. Each badge 142 includes a unique ID 166 (FIG. 5) that distinguishes that badge 142 from other badges 142. When a healthcare provider arrives at a healthcare facility, he or she typically grabs a badge 142 from a common collection of badges 142 and wears it for the duration of his or her work shift (and returns it to the general collection after his/her shift). In conventional badge servers 94, in order for server 94 to know that a particular badge 142 is assigned to a particular healthcare worker, the worker has to manually associate the particular badge 142 they chose to wear that day with their name (or some other worker ID). This manual association may involve scanning the badge on a badge reader in communication with server 94, typing information (such as the badge ID and/or the worker's ID) into a computer coupled to server 94, entering information into badge 142 (if it is a badge that allows data entry), performing other actions, and/or a combination of these steps.

Badge server 94 may also be configured to monitor the location of badges 142 within a healthcare facility. Typically this location monitoring is performed through the monitoring of the wireless access points 82 throughout the healthcare facility. That is, badges 142 are often equipped to use WiFi, or other wireless communication protocols, that allow them to communicate with wireless access points 82. By monitoring which access points the badges 142 are currently connected to, using a map of the location of the access point 82 within the facility, and, in some cases, using the signal strengths between the badges 142 and one or more of the access points 82, the general position of the badges 142 within the healthcare facility can be determined by badge server 94. Often this location determination is low resolution. That is, for example, this location-determining technique may only indicate which room the caregiver's badge 142 is currently located in, or which hallway, but might not provide accurate information as to where the badge 142 is located within a particular room or hallway. As will be discussed in greater detail below, patient support apparatuses 20 are equipped with a separate and independent location-determination technology that can be used to monitor the location of badges 142 (and/or other devices) with greater accuracy, such as locations that are accurate to within five to ten centimeters (although other accuracy levels may be utilized in accordance with this disclosure).

Badges 142 may be badges of the type sold or marketed by Stryker Corporation of Kalamazoo, Michigan, under the names Vocera Badge, Vocera Smartbadge, and/or Vocera Minibadge. Other types of badges may also, or alternatively, be used. Such badges 142 include the ability to transmit voice communications of healthcare workers to other badges 142 and/or other locations within a healthcare facility. Some of the badges may also include text messaging abilities, alarm notifications, and other functions. When integrated into the system described herein, such badges 142 may be modified to include one or more ultra-wideband transceivers and/or tags that communicate with ultra-wideband transceivers onboard patient support apparatus 20, as will be discussed in greater detail herein. That is, patient support apparatus 20 may be configured to repetitively determine the location of any of the badges 142 that are positioned within range of its ultra-wideband transceivers and determine whether the badge 142 is positioned inside or outside of one or more volumes of space, as will also be discussed in greater detail below. In some embodiments, badges 142 that do not include UWB transceivers may also be incorporated into the communication system described herein

In some embodiments, patient support apparatus server 84 (FIGS. 4 & 8) communicates with EMR server 92 in order to transmit patient data that is to be recorded in a patient's health record (e.g. vital sign readings from one or more vital sign sensors; weight readings taken from the scales built into patient support apparatuses 20; therapies provided to patients using a powered mattress 42 onboard patient support apparatuses 20; data from other devices that are determined to be associated with the patient assigned to patient support apparatus 20, etc.). In addition, server 84 communicates with EMR server 92, in some embodiments, in order to receive data from one or more of the devices that are being used with a particular patient.

It will be understood that the architecture and content of local area network 80 will vary from healthcare facility to healthcare facility, and that the examples shown in FIGS. 4 & 8 are merely two examples of the type of network a healthcare facility may be employ. Typically, one or more additional servers will be hosted on network 80 and one or more of them may be adapted to communicate with patient support apparatus server 84. Local area network 80 will also typically allow one or more electronic devices 98 to access the local area network 80 and the servers hosted thereon via wireless access points 82. Such electronic devices 98 include, but are not limited to, smart phones, tablet computers, portable laptops, desktop computers, smart televisions, and other types of electronic devices that include a WiFi capability and that are provided with the proper credentials (e.g. SSID, password, etc.) to access network 80 (and, in at least some situations, patient support apparatus server 84). As will be discussed in further detail herein, patient support apparatus server 84 is configured, in some embodiments, to share data with one or more electronic devices 98 that relates to patient support apparatus 20, that relates to one or more devices 100 that become associated with patient support apparatus 20 (or the patient assigned thereto), that relates to one or more badges 142 that become associated with patient support apparatus 20, and/or that relates to one or more medical records of the patient stored in EMR server 92.

Linked locator units 60 are adapted to wirelessly receive signals from patient support apparatus 20 and deliver the signals to communications outlet 64 in a manner that matches the way the signals would otherwise be delivered to communications outlet 64 if a conventional nurse call cable 66 were connected directly between patient support apparatus 20 (via a cable port 148; FIG. 5) and communications outlet 64. Linked locator units 60 are also adapted to transmit signals received from communications outlet 64 to patient support apparatus 20 via a BT transceiver 106 and/or a UWB transceiver 126 (FIG. 5). Thus, patient support apparatus 20 and linked locator unit 60 cooperate to send signals to, and receive signals from, communications outlet 64 in a manner that is transparent to communications outlet 64 such that outlet 64 cannot detect whether it is in communication with patient support apparatus 20 via a wired connection or it is in communication with patient support apparatus 20 via a wireless connection between patient support apparatus 20 and linked locator unit 60 (the latter of which is in wired communication with outlet 64). In this manner, a healthcare facility can utilize the wireless communication abilities of one or more patient support apparatuses 20 without having to make any changes to their existing communication outlets 64.

As noted, in addition to sending signals received from patient support apparatus 20 to communications outlet 64, linked locator units 60 are also adapted to forward signals received from communications outlet 64 to patient support apparatus 20. Linked locator units 60 are therefore adapted to provide bidirectional communication between patient support apparatus 20 and communications outlet 64. This bidirectional communication includes, but is not limited to, communicating command signals from any of controls 50 and/or from any of electronic devices 98 to corresponding room devices 72, 74, and/or 76 and communicating audio signals between a person supported on patient support apparatus 20 and a caregiver positioned remotely from patient support apparatus 20. The audio signals received by linked locator unit 60 from a microphone on patient support apparatus 20 are forwarded to communications outlet 64 (for forwarding to nurse call system 70), and the audio signals of a remotely positioned nurse that are received at communications outlet 64 (from nurse call system 70) are forwarded to a speaker onboard patient support apparatus 20.

Nurse call cable 66 (FIGS. 4 & 8), in some embodiments, includes a conventional 37 pin connector on each end, one of which is adapted to be inserted into outlet 64 and the other one of which is adapted to be inserted into a linked locator unit 60 (or cable port 148 of patient support apparatus 20 if wired communication is desired). Such 37 pin connections are one of the most common types of connectors found on existing walls of medical facilities for making connections to the nurse call system 70 and room devices 72, 74, and 76. Linked locator unit 60 and nurse call cable 66 are therefore configured to mate with one of the most common type of communication outlets 64 used in medical facilities. Such 37 pin connectors, however, are not the only type of connectors, and it will be understood that linked locator units 60 can utilize different types of connectors that are adapted to electrically couple to different types of nurse call cables 66 and/or different types of communication outlets 64. One example of such an alternative communications outlet 64 and cable 66 is disclosed in commonly assigned U.S. patent application Ser. No. 14/819,844 filed Aug. 6, 2015 by inventors Krishna Bhimavarapu et al. and entitled PATIENT SUPPORT APPARATUSES WITH WIRELESS HEADWALL COMMUNICATION, the complete disclosure of which is incorporated herein by reference. Still other types of communication outlets 64 and corresponding connectors may be utilized.

Locator unit 60 (FIG. 4) also includes an electrical cord 150 having a plug positioned at a far end that is adapted to be inserted into a conventional electrical outlet 108. Electrical cord 150 enables locator unit 60 to receive power from the mains electrical supply via outlet 108. It will be appreciated that, in some embodiments, locator unit 60 is battery operated and cord 150 may be omitted. In still other embodiments, locator unit 60 may be both battery operated and include cord 150 so that in the event of a power failure, battery power supplies power to locator unit 60, and/or in the event of a battery failure, electrical power is received through outlet 108.

In some embodiments, locator units 60 include a video port 120 that is adapted to receive a display cable 110 (FIG. 4). The display cable 110 is adapted to couple to locator unit 60 at one end and a display device 56 at its opposite send. Locator unit 60 may be configured to use cable 110 to send data to display device 56 that is to be displayed thereon. Such data may include data from one or more devices 100 that are associated with the patient on patient support apparatus 20 (or with patient support apparatus 20 itself), data from one or more badges 142, status data from one or more sensors onboard patient support apparatus 20, location data regarding the location of patient support apparatus 20, and/or other data. Cable 110 maybe a High-Definition Multimedia Interface (HDMI) cable, a Video Graphics Array (VGA) cable, a DisplayPort (DP) cable, a plurality of Radio Corporation of America (RCA) cables, a Digital Visual Interface (DVI) cable, and/or another type of cable. Locator unit 60 is configured to include a complementary type of connector that mates with a connector on an end of cable 110. Further details regarding the communication between patient support apparatus 20 and display device 56 are provided below and, in some embodiments, patient support apparatus 20 may be configured to communicate directly with certain display devices without using locator unit 60 as a communication intermediary.

In addition to the other functions described herein, locator units 60 are configured to communicate location data to patient support apparatus 20 that enables patient support apparatus 20 and/or patient support apparatus server 84 to determine the location of patient support apparatus 20 within the healthcare facility. In general, such location determination is carried out by patient support apparatus 20 analyzing wireless signals communicated between itself and locator unit 60 in order to determine its position relative to locator unit 60. If patient support apparatus 20, or a predefined reference point on patient support apparatus 20 (e.g. its head end, its center, etc.) is positioned within a threshold distance of locator unit 60, patient support apparatus 20 associates itself with the locator unit 60. When associated, patient support apparatus 20 may communicate data to locator unit 60, receive data from locator unit 60, and also deem its location within the healthcare facility to be the same as location of locator unit 60. When patient support apparatus 20 is outside of the threshold distance, it does not associate itself with locator unit 60, and therefore does not exchange data with locator unit 60 or consider its location to be the same as that of locator unit 60's location.

In some embodiments, patient support apparatus 20 is configured to associate itself with a particular locator unit 60 if controller 140 determines that the locator unit 60 is within a volume of space 152a (FIGS. 4 & 6), or locator unit 60 determines that patient support apparatus 20 (or a reference point thereon) is positioned within volume of space 152a. In some embodiments, the volume of space 152a is defined with respect to each locator unit 60 and does not move. In other embodiments, the volume of space 152a is defined with respect to patient support apparatus 20 and moves as patient support apparatus 20 moves. In some embodiments, patient support apparatus 20 associates itself with a nearby locator unit 60 if both the locator unit 60 and the patient support apparatus 20 (or a reference point thereon) are concurrently within the predefined volume of space 152a. Regardless of whether volume of space 152a is defined with respect to a locator unit 60, or with respect to a patient support apparatus 20, by at least one or both of these devices (locator unit 60 and patient support apparatus 20) being positioned within the predefined volume of space 152a, the locator unit 60 and patient support apparatus 20 will be positioned within a threshold distance of each other. An example of patient support apparatus 20 occupying a volume of space 152a is shown in FIG. 4, where head end 38 of patient support apparatus 20 is positioned inside of volume of space 152a, and patient support apparatus 20 has therefore associated itself with that particular locator unit 60.

After associating itself with a particular locator unit 60, patient support apparatus 20 is configured to be able to have its absolute position within the healthcare facility determined by receiving a unique locator identifier (ID) 122 (FIG. 5) from the locator unit 60. The location of each locator unit 60 in the healthcare facility is surveyed during the installation of locator units 60, and the unique IDs 122 of each locator unit 60 are also recorded during the installation of locator units 60. This surveying information and corresponding ID information may be stored in patient support apparatus server 84 and/or onboard the patient support apparatuses 20, thereby enabling a patient support apparatus 20 and/or patient support apparatus server 84 to determine the location of a patient support apparatus 20 once it is associated with a particular locator unit 60.

In those embodiments where patient support apparatus server 84 is configured to determine the location of patient support apparatus 20, patient support apparatus 20 sends its relative position information with respect to the associated locator unit 60, and/or the ID 122 of the associated locator unit 60 (and its own unique patient support apparatus ID 130 (FIG. 5)) to server 84. Server 84 includes a table of all of the locations of the locator units 60 (which, as noted, is generated via a surveying operation during the installation of locator units 60), and it uses that table to correlate the patient support apparatus IDs 130 and the locator unit IDs 122 it receives to specific locations within the healthcare facility. Thus, if a particular patient support apparatus 20 (with a particular ID 130) sends to server 84 an associated locator unit ID 122 that corresponds to room 430, server 84 determines that that particular patient support apparatus 20 is currently located in room 430. Generally speaking, and as will be discussed in greater detail below, the location of a patient support apparatus 20 is deemed to correspond to whichever locator unit 60 it is currently associated with, and if it is not currently associated with any locator unit 60, its location may be considered to be indeterminate.

In some embodiments of patient support apparatuses 20 and locator units 60, the relative location of a patient support apparatus 20 to a locator unit 60 is determined solely using ultra-wideband communication between the patient support apparatus 20 and the locator unit 60. Alternatively, in some embodiments, patient support apparatus 20 solely uses short range infrared communications with locator unit 60 to determine its relative location, wherein such short range infrared communications are only possible when the patient support apparatus 20 is positioned within a close proximity to the locator unit 60 (e.g. in the range of about 1-3 unobstructed meters). In these latter embodiments, patient support apparatus 20 may report that its location coincides with that of the nearby locator unit 60 when it is able to successfully communicate with the nearby locator unit 60 using these short range infrared communications. Still further, in some embodiments, patient support apparatus 20 and locator unit 60 may communicate with each other using both infrared and ultra-wideband communications. Further details regarding the use of short range infrared communications for location determination are described in commonly assigned U.S. Pat. No. 9,999,375 issued Jun. 19, 2018, to inventors Michael Hayes et al. and entitled LOCATION DETECTION SYSTEMS AND METHODS, the complete disclosure of which is incorporated herein by reference.

In some embodiments, locator units 60 and/or patient support apparatuses 20 may be constructed to include any or all of the functionality of the wireless headwall units and/or patient support apparatuses disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION; in commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION; and/or in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosures of all of which are incorporated herein by reference.

Still further, in some embodiments, locator units 60 and/or patient support apparatuses 20 may be constructed to include any of the features and/or functions of the headwall units 144a and/or patient support apparatuses disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference.

FIG. 5 depicts a block diagram of patient support apparatus 20, a linked locator unit 60, a device 100, a badge 142, a display device 56, and network 80. As will be discussed in greater detail below, patient support apparatus 20 is configured to automatically determine the location of one or more locator units 60, badges 142, and/or other devices 100 that either have built-in UWB transceivers or a UWB tag attached to them. In addition, patient support apparatus 20 is configured to automatically carry out communications with these devices if they are positioned within a defined proximity to patient support apparatus 20. In some embodiments, if a particular device is positioned within the defined proximity, patient support apparatus 20 automatically associates the device with the patient assigned to patient support apparatus 20 (and/or with patient support apparatus 20 itself), and causes data from that device (or devices) to be displayed on one or more of display devices 56, and/or forwards data from that device (or devices) to patient support apparatus server 84 and/or EMR server 92 in communication with network 80. When the device is positioned outside the defined proximity, patient support apparatus 20 may automatically disassociate itself from the device and, in some situations, terminate communications with the device and/or inform patient support apparatus server 84 of the disassociation.

Linked locator unit 60 (FIG. 5) includes an ultra-wideband transceiver 126, a Bluetooth transceiver 106, a locator unit controller 112, configuration circuitry 114, a television controller 116, a headwall interface 118, a video port 120, a unit ID 122, and, in some embodiments, an infrared transceiver 124. Bluetooth transceiver 106 is adapted to communicate with a Bluetooth transceiver 128 onboard patient support apparatus 20 using RF waves in accordance with conventional Bluetooth standards (e.g. IEEE 802.14.1 and/or any of the standards maintained by the Bluetooth Special Interest Group (SIG) of Kirkland, Washington, USA). In some embodiments, transceivers 106 and 128 utilize Bluetooth Low Energy communications.

Ultra-wideband transceiver 126 is adapted to communicate with one or more ultra-wideband transceivers 132 positioned onboard patient support apparatus 20. Transceiver 126 is adapted to determine a distance between itself and patient support apparatus 20. Alternatively, or additionally, transceiver 126 may be adapted to allow one or more of the UWB transceivers 132 onboard patient support apparatus 20 to determine their distance(s) from transceiver 126. In some embodiments, transceivers 126 and 132 use time of flight (TOF) computations to determine these distances. In other embodiments, transceivers 126 and 132 may utilize other techniques (e.g. time difference of arrival, two-way ranging, angle of arrival, channel state information, etc.) for determining their distances from each other, either in addition to, or in lieu of, TOF computations. In some embodiments, transceivers 126, 132 may also determine an angle between themselves using angular information derived from antenna arrays positioned onboard transceivers 126, 132, or by using other techniques. The position and orientation of each transceiver 132 onboard patient support apparatus 20 is known and stored in an onboard memory 134 and used to determine the position and orientation of patient support apparatus 20 with respect to the locator unit(s) 60 with which it is communicating. Such position and orientation information may be determined using conventional trilateration and/or triangulation techniques, or other techniques.

In some embodiments, transceivers 126, 132 are implemented as any of the Trimension™ ultra-wideband modules available from NXP Semiconductors of Austin, Texas. These modules include, but are not limited to, the Trimension™ UWB modules ASMOP1BO0N1, ASMOP1CO0R1, and/or the ASMOP1CO0A1, that utilize any of the following chips: the NXP SR150, SR100T, SR040, NCJ29D5, and/or the OL23DO chips. Modules manufactured and/or marketed by other companies may also be used, including, but not limited to, the Decawave DWM1000, DWM10001C, DWM3000 modules (available from Decawave of Dublin, Ireland); the Nordic TSG5162 SiP module (available from Tsingoal Technology of Beijing, China); and/or the UWB hub, wand, and/or sensors available from Zebra technologies of Lincolnshire, Illinois. Still other types of UWB modules may be used to implement transceivers 126 and 132.

Locator unit controller 112 is adapted to control the operation of transceivers 126, 106, configuration circuitry 114, TV controller 116, headwall interface 118, video port 120, and, if included, IR transceiver 124 (FIG. 5). When infrared transceiver 124 is included, it may be included to provide backwards compatibility to patient support apparatuses 20 that are not equipped with a UWB transceiver 132. That is, some healthcare facilities may include one or more patient support apparatuses that are not equipped with a UWB transceiver 132, but that do include an IR transceiver that is adapted to communicate with IR transceiver 124. When locator unit 60 includes IR transceiver 124, it is able to communicate its unit ID 122 to such patient support apparatuses via IR transceiver 124, which is a short range transceiver that is configured to only communicate with an adjacent patient support apparatus when the patient support apparatus is nearby (e.g. without about five feet or so). Such an adjacent patient support apparatus 20 then communicates the received locator unit ID 122 along with its own unique ID 130 (FIG. 5) to server 84 which, as noted previously, is able to correlate the locator unit ID 122 to a particular location with the healthcare facility. In this manner, server 84 is able to use locator units 60 determine the location of versions of patient support apparatuses 20 that don't have a UWB transceiver 132, but that do have an IR transceiver.

Headwall interface 118 is adapted to change the electrical state of one or more pins that are in electrical communication with communication outlet 64 (via cable 66). Headwall interface 118 changes these electrical states in response to instructions from controller 112. For example, if the exit detection system 136 of patient support apparatus 20 detects a patient exit, a controller 140 of patient support apparatus 20 sends an exit alert signal to linked locator unit 60 and controller 112 responds by instructing headwall interface 118 to change the electrical state of at least one pin that is used to signal an exit alert (or a generic priority alert) to the nurse call system 70 via communications outlet 64. Additionally, if a device 100, such as a portable exit detection sensor, is associated with patient support apparatus 20 and it detects a patient exit, the exit detection sensor may transmit an exit detection alert signal to patient support apparatus 20, which in turn forwards the exit alert signal to linked locator unit 60, and controller 112 responds by instructing headwall interface 118 to change the electrical state of the same pin or pins that it does in response to receiving an exit detection alert from exit detection system 136.

In some embodiments, headwall interface 118 may be constructed in the same manner as, and/or may include any one or of the functions as, the cable interface 88 described in commonly assigned U.S. patent application Ser. No. 63/193,778 filed May 27, 2021, by inventors Krishna Bhimavarapu et al. and entitled PATIENT SUPPORT APPARATUS AND HEADWALL UNIT SYNCING, the complete disclosure of which is incorporated herein by reference. Alternatively, or additionally, headwall interface 118 may be constructed in the same manner as, and/or may include any one or more of the same functions as, the headwall interface 120 disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference. Linked locator unit 60 may also be configured to perform any of the functions of the headwall units 94 disclosed in the above-mentioned '778 patent application.

Configuration circuitry 114 and TV controller 116 may be configured to perform any of the same functions as, and/or be constructed in any of the same manners as, the configuration circuitry 132 and the TV control circuit 134, respectively, of commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which has already been incorporated herein by reference. Additionally, or alternatively, linked locator unit 60 may be configured to perform any of the functions of the headwall units 144 disclosed in the aforementioned '508 patent application.

Patient support apparatus 20 includes a controller 140, a memory 134, exit detection system 136, a scale system 144, monitoring system 138, a microphone 146, Bluetooth transceiver 128, one or more UWB transceivers 132, display 52 (which may be part of control panel 54a, and/or another control panel 54), network transceiver 96, a nurse call interface 154, a brake 300, a battery 304, an emergency button (e-button) 302, and a plurality of additional components that are not shown in FIG. 5. Patient support apparatus 20 may also include a plurality of additional components that are not shown in FIG. 5. In other embodiments, patient support apparatus 20 may include different a different combination of some or all of the components shown in FIG. 5 and/or other components not shown therein. In some embodiments, patient support apparatus 20 may omit one or more of the components shown in FIG. 5.

Each UWB transceiver 132 is positioned at a known location on patient support apparatus 20. This known location information is stored in memory 134 and/or elsewhere, and may be defined with respect to any suitable frame of reference that is common to patient support apparatus 20. The known location information may include the spatial relationship between UWB transceivers 132 and/or any other components of patient support apparatus 20. For example, in some embodiments, the known location information includes the spatial relationship not only between UWB transceivers 132, but also the spatial relationships between UWB transceivers 132 and one or more of the following: the head end 38 of patient support apparatus 20, the foot end 40 of patient support apparatus 20, the sides of patient support apparatus 20, a reference point defined on patient support apparatus 20, the floor, and/or other components and/or landmarks of patient support apparatus 20. In some embodiments, this location information is used to determine the orientation of patient support apparatus 20 with respect to one or more walls 62, locator units 60, another patient support apparatus 20, and/or another object or structure within the healthcare facility.

In some embodiments, patient support apparatus 20 includes four UWB transceiver 132, each of which are positioned generally adjacent one of the four corners of patient support apparatus 20. In some such embodiments, the four UWB transceiver 132 are attached to, or positioned near, the four corners of litter frame 28. In other embodiments, the four UWB transceivers 132 are attached to, or positioned near, the four corners of base 22. In some embodiments, each of the four UWB transceivers 132 are attached to the corners of support deck 30. Still other locations of the UWB transceivers 132, as well as different numbers of the UWB transceiver 132, may be incorporated into patient support apparatus 20. In those embodiments of patient support apparatus 20 where one or more of the UWB transceivers 132 are coupled to components of patient support apparatus 20 that are movable (e.g. litter frame 28, which can have its height and orientation changed; or support deck 30 that can have its sections, such as head section 44, pivoted), sensors are included within patient support apparatus 20 that communicate the current position of the movable component to controller 140 so that controller 140 is able to determine the current positions of the UWB transceivers 132 and use those positions when determining the current location of a device, such as a badge 142 and/or another device 100.

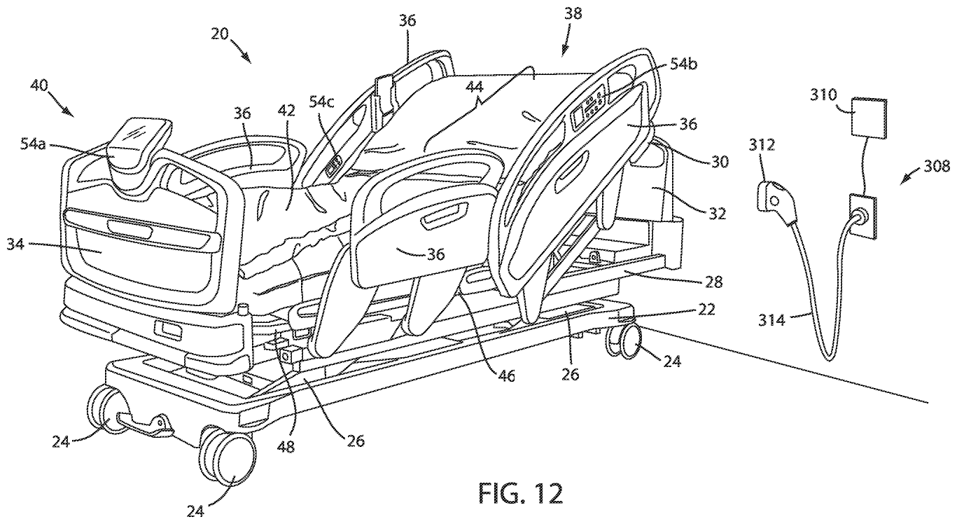

In some embodiments, patient support apparatus 20 includes one or more batteries 304 (FIG. 5) that are adapted to supply electrical power to patient support apparatus 20 when its power cord 150 (FIGS. 4 & 8) is not coupled to an electrical outlet. Battery 304 is rechargeable battery of a conventional type. Battery 304 may be re-charged by plugging a power cord 150 (FIGS. 4 & 8) of patient support apparatus 20 into an electrical outlet, or, in some embodiments, by connecting patient support apparatus 20 to a charging station 308 (FIG. 12). As will be discussed in greater detail below, the charge level and/or other characteristics of battery 304 is monitored by controller 140 and shared, as appropriate, with server 84 and/or one or more caregiver badges 142, thereby enabling healthcare personnel about the status of battery 304.

E-button 302 is a button that is integrated into patient support apparatus 20 and that is designed to be pressed by a patient or caregiver when an urgent need for help arises. In some embodiments, patient support apparatus 20 includes an e-button 302 on one or more of its patient control panels 54c and/or on one or more of its caregiver control panels 54b. E-button(s) 302 may also be positioned elsewhere on patient support apparatus 20. In some embodiments, e-button 302 is configured, when pressed, to send a request for help using one or more UWB transceivers 132 to any badges 142 that are positioned within communication range of patient support apparatus 20. In such embodiments, the request for help is sent directly to the badges 142 via UWB communications without passing through an intermediate devices. In some embodiments, e-button 302 is configured, when pressed, to also, or alternatively, send a request for help to patient support apparatus server 84 via network transceiver 96 and one or more wireless access points 82. In response to receipt of this message, server 84 may send notification(s) to one or more badges 142 via wireless access point 82. Server 84 may select one or more recipient badges 142 based upon their current location and/or based on other criteria, such as who is or is not assigned to care for the particular patient requesting help, the skills or the particular caregivers associated with badges 142, and/or other criteria.

In those embodiments where controller 140 sends a request for help via one or more WUB transceivers 132 in response to a user pressing on e-button 302, controller 140 is configured to also send location information with the request for help. The location information, which may correspond to a room number, is determined by controller 140 through communications with a nearby locator unit 60 and/or through communications with server 84. The message sent requesting help therefore also informs the recipient(s) of the location of the person who activated the e-button 302, thereby informing the recipient where they need to go to provide the requested help.

Controller 140 (FIG. 5), as well as controller 112, may take on a variety of different forms. In the illustrated embodiment, each of these controllers is implemented as a conventional microcontroller. However, these controllers may be modified to use a variety of other types of circuits-either alone or in combination with one or more microcontrollers-such as, but not limited to, any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by controllers 112 and 140 when carrying out the functions described herein, as well as the data necessary for carrying out these functions, are stored in a corresponding memory that is accessible to that particular controller (e.g. memory 134 for controller 140, and a memory (not shown) for controller 112). In some embodiments, controller 140 may include and/or work with a microcontroller that is integrated into, or associated with, UWB transceiver(s) 132, and controller 112 may include and/or work with a microcontroller that is integrated into, or associated with, UWB transceiver 126.

Controller 140 utilizes UWB transceivers 132 to determine the relative position of patient support apparatus 20 with respect to one or more nearby locator units 60, one or more devices 100, and/or one or more badges 142. If patient support apparatus 20 is positioned within range of a locator unit 60, its UWB transceivers 132 communicate with the UWB transceiver 126 positioned on that locator unit 60, and the transceivers 132 and 126 exchange signals that enable them to determine the distances between themselves. This distance determination is done for each UWB transceiver 132 positioned onboard patient support apparatus 20 (or for as many as is necessary in order to determine an accurate position of locator unit 60 relative to patient support apparatus 20).

In some embodiments, UWB transceivers 126, 132 may also be configured to determine an angular relationships between themselves. The distance (and angle information) in at least some embodiments is calculated by UWB transceiver 132 and controller 140 of patient support apparatus 20. In other embodiments, UWB transceiver 126 and controller 112 may calculate the distance (and angle information) and forward the results of this calculation to patient support apparatus 20 (either via UWB transceiver 126 or BT transceiver 106). In either situation, patient support apparatus controller 140 is informed of the distances (and, in some embodiments, as noted, the angle information) between transceivers 132 and 126. These distances and orientations are then used to calculate a relative position of patient support apparatus 20 to the locator unit 60 in a common frame of reference that may be defined in a fixed relationship to the patient support apparatus 20 or in a fixed relationship to the locator unit 60.

Although FIGS. 4 and 5 only illustrate a single locator unit 60, it will be understood that a typical healthcare facility will include multiple locator units 60 positioned at different locations throughout the facility, including ones positioned within patient rooms and others positioned outside of patient rooms. Typically, at least one locator unit 60 will be positioned in each patient room of the healthcare facility, and if the patient room is intended to be occupied by more than one patient (e.g. it includes multiple bays), then additional locator units 60 may be included so that each patient support apparatus 20 will have a locator unit 60 positioned adjacent to each bay area in the room. Additional locator units 60, such as unlinked locator units 60a, may also be positioned at other locations through the healthcare facility.

The location of patient support apparatus 20 relative to locator units 60 is repetitively determined by an exchange of signals between UWB transceivers 126 and 132. This exchange is initiated by an interrogation signal that may be sent by the UWB transceivers 126 of the locator unit 60, and/or it may be sent by the UWB transceivers 132 of the patient support apparatuses 20. The trigger for sending these interrogation signals (from either source) may simply be the passage of a predefined interval of time, in at least some embodiments. That is, in some embodiments, patient support apparatus 20 and/or locator units 60 may be configured to periodically send out an interrogation signal that will be responded to by any UWB transceivers 126 or 132 that are positioned with range of that signal. In those embodiments where patient support apparatuses 20 are configured to send out such an interrogation signal, the time intervals between the interrogation signals may be varied depending upon the location, the number of devices 100 and/or badges 142 (if any) that are positioned within range of patient support apparatus 20, and/or the status of the patient support apparatus 20. For example, in some embodiments, controller 140 may be configured to send out the interrogation signals with longer timer intervals between them when the patient support apparatus is stationary (and, in some cases, when no devices 100 or badges 142 are currently positioned in communication range), and to send out the interrogation signals with shorter time intervals between them when the patient support apparatus 20 is in motion and/or when at least one device 100 or badge 142 is currently positioned within communication range of transceivers 132. In any of the aforementioned embodiments, motion of the patient support apparatus 20 may be detected in any suitable manner, such as by including one or more motion sensors on the patient support apparatus 20 (e.g. one or more accelerometers), and/or by monitoring the values of the repetitive distance measurements and looking for changes indicative of movement.

The measured distances (and/or angular information between locator units 60 and patient support apparatuses 20) that are generated from the communications between UWB transceivers 126, 132 may utilize Angle of Arrival (AoA) information, Time of Flight (TOF) information, Channel State Information, Time Difference of Arrival (TDoA) information, Two-Way Ranging (TWR) ranging information, and/or other information. In some embodiments, each transceiver 126, 132 includes an array of antennas that are used to generate distance and/or angular information with respect to the transceivers 126, 132 in which it is in communication. Still further, in some embodiments, UWB transceivers 126, 132 include one or more of their own microcontrollers, and the location of UWB transceivers 126, 132 may be determined by these internal microcontrollers without utilizing controller 140 and/or 112. In other embodiments, controllers 112 and/or 140 may work in conjunction with the microcontrollers of transceivers 126, 132 to determine their relative locations to each other.

Nurse call interface 154 of patient support apparatus 20 (FIG. 5) includes Bluetooth transceiver 128 and a cable port 148, in some embodiments. Nurse call interface 154 provides an interface for patient support apparatus 20 to communicate with outlet 64 of nurse call system 70. That is, nurse call interface 154 provides the means for patient support apparatus 20 to bidirectionally communicate with communication outlet 64. As has been discussed, in some situations, patient support apparatus 20 uses Bluetooth transceiver 128 to communicate with Bluetooth transceiver 106 of linked locator unit 60, and linked locator unit 60 forwards communications back and forth between outlet 64 and patient support apparatus 20. In other words, in some situations, linked locator unit 60 functions as a communications intermediary between nurse call interface 154 and outlet 64. Alternatively, a nurse call cable 66 may be coupled directly between patient support apparatus 20 and wall outlet 64, thereby avoiding the need to use linked locator unit 60 as a communication intermediary. In such situations, one end of a nurse call cable 66 is plugged into cable port 148 of patient support apparatus 20 and the other end of the cable 66 is plugged directly into outlet 64. Nurse call interface 154 thereby provides patient support apparatus 20 with the ability to communicate either wirelessly or via wired means with the outlet 64.

Patient support apparatus 20 also includes, in at least some embodiments, a microphone 146 (FIG. 5) that is used to detect the voice of the patient when the patient wants to speak to a remotely positioned nurse. The patient's voice is converted to audio signals by microphone 146 and controller 140 is adapted to forward these audio signals to an adjacent communications outlet 64 positioned in wall 62 (FIG. 4). When a cable 66 is coupled between cable port 148 of patient support apparatus 20 and outlet 64, controller 140 forwards these audio signals to outlet 64 via the cable 66. When no such cable 66 extends between patient support apparatus 20 and outlet 64, controller 140 wirelessly forwards these audio signals to the linked locator unit 60 that it is currently associated with (using transceiver 128, or in some embodiments, one of transceivers 132) and controller 112 of linked locator unit 60 forwards these audio signals to outlet 64. As was noted, outlet 64 is in electrical communication with a conventional nurse call system 70 that is adapted to route the audio signals to the correct nurse's station 78, and/or other location. In some embodiments, microphone 146 acts as both a microphone and a speaker. In other embodiments, a separate speaker may be included in order to communicate the voice signals received from the remotely positioned nurse. In some embodiments, the audio communication between patient support apparatus 20 and communications outlet 64 is carried out in any of the manners, and/or includes any of the structures, disclosed in commonly assigned U.S. patent application Ser. No. 16/847,753 filed Apr. 14, 2020, by inventors Alexander Bodurka et al. and entitled PATIENT SUPPORT APPARATUSES WITH NURSE CALL AUDIO MANAGEMENT, the complete disclosure of which is incorporated herein by reference.

After the installation of locator units 60 in a particular healthcare facility, the location of each locator unit 60 within that facility is recorded. In some embodiments, the coordinates of the locations of locator units 60 are recorded in a common frame of reference (or converted to a common frame of reference after recordation). Such coordinates may be three dimensional (i.e. include a vertical and two horizontal components), or they may be two dimensional (no height component). In other embodiments, a more generalized location of one or more locator units 60 is determined, rather than the precise coordinates of the locator units 60. The generalized location of the locator units 60 may include an indication of the room, bay, area, hallway, portion of a hallway, wing, maintenance area, etc. that the locator unit 60 is positioned in. In still other embodiments, the locations of one or more locator units 60 are determined both generally and more precisely.

Regardless of how the location of each locator unit 60 is initially determined after they are installed in a healthcare facility (e.g. whether their coordinates are determined or a more generalized location is determined), the locations of all of the locator units 60, as well as their unique IDs 122, are stored in a memory accessible to server 84. Server 84 then uses this location data and ID data to determine the location of a patient support apparatus 20 (as well as the location of associated devices 100 and badges 142). Alternatively, or additionally, the location data and IDs 122 are forwarded to patient support apparatuses 20 for storage in their onboard memories 134 and for use in determining their own locations. In some embodiments, the location of each locator unit 60 (whether specific and/or general) may also, or alternatively, be stored in a memory within that particular locator unit 60 and shared with the devices it communicates with (e.g. patient support apparatuses 20). In some other embodiments, the location of each locator unit 60 may be stored in multiple locations.

It will be appreciated that patient support apparatuses 20 are configured to communicate with locator units 60 regardless of the orientation of the patient support apparatus 20. That is, the UWB transceivers 126 and 132 are radio frequency transceivers that do not rely on line of sight communication, unlike the IR transceiver 124 (if present). Thus, the patient support apparatuses 20 do not have to be pointed in any particular direction with respect to the locator units in order for transceivers 126 and 132 to communicate. This differs from some prior art systems that use IR communication between the patient support apparatuses 20 and the locator units and that require the IR transceiver onboard the patient support apparatus to be aimed toward the locator unit in order for communication to be established. It will also be understood that locator units 60 can be positioned on walls, columns, ceilings, or any other fixed structures within the healthcare facility.

Patient support apparatus 20 is also configured to use UWB transceivers 132 to repetitively determine the position of other devices relative to patient support apparatus 20, such as one or more badges 142 and/or other devices 100 (FIG. 5). As will be discussed in greater detail below, controller 140 uses UWB transceivers 132 to determine the relative position of these devices by communicating with one or more respective UWB transceivers that are either built into those devices or attached to a tag that is affixed to those devices. Such UWB transceivers operate in the same manner as UWB transceivers 132 and/or UWB transceiver 126 of locator units 60. And, as will also be discussed in greater detail below, controller 140 of patient support apparatus 20 uses the relative position information to determine how it will interact with these devices, including whether to associate with these devices or not. When controller 140 associates patient support apparatus 20 with one or more of these devices, as will be discussed in greater detail below, controller 140 and/or server 84 may take one or more of the following actions: display data from these devices on display 52 and/or display device 56; send data from one or more of these devices to patient support apparatus server 84, EMR server 92, and/or badge sever 94 via network transceiver 96; retrieve data about one or more of these devices from patient support apparatus server 84, EMR server 92, and/or badge sever 94 via network transceiver 96; send one or more signals from these devices to communication outlet 64 (via a cable 66 or through linked locator unit 60); forward one or more signals from outlet 64 to one or more of these devices; retrieve data from EMR server 92 that was generated by these devices; retrieve data from these devices via another route that is independent from EMR server 92; and/or take other actions.

In the particular embodiment shown in FIG. 5, badge 142 includes a controller 156, a UWB transceiver 158, a microphone 160, a speaker 162, a network transceiver 164, and a unique ID 166. UWB transceiver 158 is adapted to communicate with the UWB transceivers 132 positioned onboard patient support apparatus 20 so that the position of badge 142 relative to patient support apparatus 20 can be repetitively determined. UWB transceiver 158 may be the same as all of the other UWB transceivers discussed herein (e.g. UWB transceivers 132, 126, etc.). UWB transceiver 158 is further adapted to transmit the unique ID 166 of badge 142 to patient support apparatus 20 so that patient support apparatus 20 knows which specific badge 142 it is communicating with. It will be understood that the components of badge 142 shown in FIG. 5 are merely an illustrative example of a badge 142, and that different badges may be utilized with the communication system of the present disclosure that have fewer, greater, or different components than those shown in FIG. 5. Those badges 142 that include a UWB transceiver 158 and identifier 166—which may be integrated into the badge 142 or part of a tag that is attached to the badge—will be able to fully operate in accordance with the principles discussed herein. However, badges that do not include a UWB transceiver 158 can still be integrated with the communications of the patient support apparatuses 20 and/or patient support apparatus server 84.

Controller 156 of badge 142 is adapted to oversee the operation of badge 142, process the communications of UWB transceiver 158 with other UWB transceivers (e.g. transceivers 132), respond to the activation of controls on badge 142 (if any), and oversee the operation of microphone 160 and speaker 162. Badge 142 is designed to be worn by a healthcare facility worker, or attached to a structure (e.g. necklace, bracelet, clip, etc.) that is adapted to be worn by a healthcare facility worker. Badge 142 of FIG. 5 is adapted to allow a caregiver to speak into microphone 160 and have his or her voice transmitted to another badge 142, and/or a computing device in communication with badge server 94. Similarly, badge 142 of FIG. 5 is adapted to receive audio signals from other badges 142, or computing devices, and to route them to speaker 162 so that the person wearing badge 142 can hear those audio signals. In other words, badge 142 is adapted to allow its wearer to communicate with remotely positioned personnel who are either wearing another badge 142 or who are able to access badge server 94 and communicate audio signals to/from server 94 using a conventional computing device (e.g. a desktop computer with a headset having a microphone and speakers).

Badge 142 communicates audio signals by routing them through badge server 94 using network transceiver 164. Network transceiver 164, like network transceiver 96 of patient support apparatus 20, may be a WiFi transceiver (e.g. IEEE 802.11) that wirelessly communicates with one or more conventional wireless access points 82 of local area network 80. In other embodiments, network transceiver 164 may be a wireless transceiver that uses conventional 5G technology to communicate with network 80 and badge server 94. In still other embodiments, network transceiver 164 may be a cellular transceiver that is adapted to use one or more protocols of conventional cell phones to communicate with badge server 94.

Regardless of the specific technology of network transceiver 164, when a user of a badge 142 calls another caregiver, controller 156 sends the user's audio signals to badge server 94 and badge server 94 routes those audio signals to the appropriate recipient (which, as noted, may be another badge or a computing device in communication with badge server 94). Similarly, the recipient's audio signals are routed to badge server 94 and badge server 94 then forwards those to badge 142. In this manner, two-way voice communication is established between a first badge 142 and another badge 142 or computing device.

Controller 140 of patient support apparatus 20 repetitively determines the position of badges 142 after they move within communication range of UWB transceivers 132. Controller 140 uses UWB transceivers 132, which communicate with the UWB transceiver 158 of each badge 142, to repetitively determine the relative position of the badge 142 with respect to patient support apparatus 20. Controller 140 compares this relative position to a space volume 152c (FIGS. 4 & 6) and, if it is outside of space volume 152c, it automatically disassociates badge 142 from patient support apparatus 20. On the other hand, as long as the position of badge 142 remains inside of space volume 152c, controller 140 maintains the association of badge 142 with patient support apparatus 20. As will be discussed in greater detail below, once a badge 142 is associated by controller 140 with a patient support apparatus 20, controller 140 may take one or more automatic actions, several of which will now be discussed.

In some embodiments, after controller 140 associates a badge 142 with patient support apparatus 20, it is configured to automatically determine a class of user to which the badge 142 belongs, and/or to send the badge ID 166 to patient support apparatus server 84 to patent support apparatus server 84 to determine the corresponding user class. That is, the unique ID 166 of each badge 142 may contain information that indicates which class of healthcare worker the badge 142 belongs to. Alternatively, it may be necessary to determine the class of worker to which a badge 142 belongs by utilizing the identity of the caregiver who is currently using that badge 142. In such cases, patient support apparatus server 84 is configured to receive information from one or more other servers on network 80 that assign classes to the healthcare workers within that particular healthcare facility. Such servers may include, but are not limited to caregiver assignment server 104 and/or another server. By combining these healthcare worker identities with the badge identifiers 166, along with data from badge server 94 that correlates badge identifiers 166 with healthcare worker identities, patient support apparatus server 84 is able to determine which class of healthcare worker a given badge 142 corresponds to.

The different classes of workers who wear badges 142 may vary from healthcare facility to healthcare facility. In one embodiment, the different classes of users may correspond to nurses, nursing assistants, doctors, service technicians, cleaning personnel, and transportation assistants (the latter of which are responsible for transporting patients to different locations within the healthcare facility). Server 84 may also be customizable to allow authorized personnel of a healthcare facility to define their own classes, and in some instances, such personnel may further refine one or more of the aforementioned classes. For example, some healthcare facilities may assign classes to certain types of doctors and/or nurses, such as emergency room doctors and/or nurses, pediatric doctors and/or nurses, cardiac doctors and/or nurses, etc. Other classification systems can, of course be used. Also, as noted above, the classification of healthcare workers may be defined on another server within the healthcare facility and read by patient support apparatus server 84.

Regardless of the specific classifications that are implemented in a healthcare facility, controller 140 and/or patient support apparatus server 84 are configured to determine which class each badge 142 belongs to using the badge ID 166 and the other information mentioned above (a badge-to-worker-ID database and a worker-ID-to-worker-class database). After making this determination, in some embodiments, controller 140 and/or patient support apparatus server 84 are configured to make one or more changes to patient support apparatus 20 in response to that determination. For example, in some embodiments, controller 140 may automatically display a care plan, or a portion of a care plan, on display 52 and/or display device 56 in response to associating with a badge 142 that belongs to a caregiver class. In such embodiments, controller 140 may be configured to not automatically display the care plan, or portion of a care plan, if the associated badge 142 belongs to a different class of users beside caregivers, such as technicians, cleaning personnel, and/or transportation assistants. In this manner, people who are not responsible for implementing the patient's care plan are not automatically presented with data regarding that care plan when they move within space volume 152c, while people who are responsible for implementing the patient's care plan are automatically presented with data regarding that care plane when the move within space volume 152c.

Controller 140 may also be configured to automatically cease displaying the care plan, or portions of the care plan, when the badge 142 of a caregiver moves outside of space volume 152c. By automatically stopping the display of this care plan information, other individuals (who may or may not be wearing a badge 142) who enter the patient's room, or otherwise move close to patient support apparatus 20, are not automatically presented with this care plan information. This helps preserve the confidentiality of the patient's care plan.

The automatic displaying of a care plan, or portion of a care plan, saves the caregiver the effort of having to manually manipulate one or more controls 50 on patient support apparatus 20 (or another device) to see the patient's care plan information. Instead, as soon as the caregiver (who is wearing a caregiver badge 142) enters a patient's room (or otherwise moves within space volume 152c), controller 140 is configured to automatically retrieve care plan information relating to the patient assigned to patient support apparatus 20 and display it for the caregiver to see. This not only saves the caregiver the manual effort otherwise required to retrieve and display this information, it also automatically presents a visual reminder to the caregiver of the care plan for this particular patient, thereby reinforcing the care plan to the caregiver and making it easier for him or her to implement the care plan with the patient.

In many situations, the care plan for a particular patient is stored in EMR server 92. In some embodiments, patient support apparatus 20 and/or patient support apparatus server 84 may also, or separately, store all or a portion of the patient's care plan. To the extent the care plan is stored onboard patient support apparatus 20, controller 140 simply retrieves it from memory 134 and display all or a portion of it in response to a caregiver badge 142 coming within space volume 152c. Similarly, if the patient's care plan is stored in patient support apparatus server 84, controller 140 simply requests this care plan from server 84 (via network transceiver 96) in response to caregiver badge 142 coming within space volume 152c. In such situations, patient support apparatus server 84 stores the care plans for patients in a manner that associates each care plan with a specific patient support apparatus 20, so that when a particular patient support apparatus 20 requests the patient's care plan, it can automatically forward the correct patient care plan to the correct patient support apparatus 20.

In those situations where the care plan is stored, either partially or wholly on EMR server 92 (or another server besides patient support apparatus server 84), controller 140 and/or server 84 needs to determine the patient assigned to patient support apparatus 20 before they can request the care plan information directly from EMR server 92. In order to determine who the particular patient is who is assigned to patient support apparatus 20, controller 140 and/or server 84 communicates with ADT server 102. ADT server keeps track of which patients are assigned to which rooms and/or which bays of rooms with multiple patients. In order for controller 140 or server 84 to determine which patient is assigned to a particular patient support apparatus 20, they need only to determine which room and/or bay that particular patient support apparatus 20 is currently located in. This is accomplished through communications with an associated locator unit 60, as discussed above. In brief overview, controller 140 either sends the locator unit ID 122 of an associated locator unit 60 to patient support apparatus server 84, which then looks up the room and/or bay location of that locator unit ID 122, or controller 140 consults an onboard listing of what rooms and/or bays correspond to each particular locator unit 60. In either case, the room or bay number of a particular patient support apparatus 20 is determined. After determining which room or bay a particular patient support apparatus is located in, ADT server 102 is consulted to determine the particular patient assigned to that particular room or bay (and thus that particular patient support apparatus 20). After determining the particular patient, controller 140 and/or server 84 are configured to use the patient's identity to retrieve the patient's care plan from EMR server 92. EMR server 92 either delivers the care plan directly to controller 140, or it delivers it to patient support apparatus server 84 first, and server 84 then forwards it to controller 140. As noted, controller 140 may then display all or portions of it on display 52 and/or one or more display device 56 that are associated with that particular patient support apparatus 20.

It will be understood that patient support apparatus 20 and/or server 84 are configurable, in some embodiments, such that authorized personnel can customize what information, if any, of a patient's care plan is automatically displayed by controller 140 in response to a caregiver's badge 142 becoming associated with a patient support apparatus 20. This customization may be carried out using caregiver control panel 54c on patient support apparatus 20 and/or a computing device that is in communication with patient support apparatus server 84. This customization also allows authorized personnel to completely shut off the automatic display of care plan information in response to caregiver badges becoming associated with a particular patient support apparatus 20.

The customization of the automatic display of care plan information can also be changed by authorized personnel based on other factors, such as particular locations within the healthcare facility, the time of day, particular days of the week or month, particular classes of users, particular types of patients (e.g. type of illness or condition they are being treated for), and/or other factors. In this manner, authorized personnel can, for example, have care plan information automatically displayed for certain rooms within the healthcare facility, but not others; for certain caregivers, but not others; for certain patients, but not others; at certain times of the day, week, or month, but not others, etc. The customization of the care plan display may also be extended to the contents of the care plan. That is, patient support apparatus 20 and/or server 84 are configured, in some embodiments, to allow authorized users to select what aspects of the care plan are going to be automatically display and what aspects, if any, are not going to be automatically displayed.

The contents of a patient's care plan can vary from healthcare facility to healthcare facility. Patient support apparatus 20 and server 84 are configured to be able to automatically display all or portions of the care plan, regardless of how a particular healthcare facility populates the contents of the care plan. In some situations, a healthcare facility may populate the care plan with information such as one or more of the following: medications that are to be administered to a patient; mattress therapies that are to be administered to the patient; patient turns that are to be implemented with the patient; one or more steps for preventing or mitigating patient bed sores; one or more steps for preventing or mitigating patient falls; mobility activities that are to be implemented with the patient (e.g. patient should be moved out of patient support apparatus 20 at certain times and/or a certain number of times each day, etc.); tests that are to be given to the patient; treatments that are to be administered to the patient; and/or other information.

In addition to, or in lieu of, care plan information, controller 140 may be configured to display other information automatically in response to a caregiver badge 142 entering space volume 152c. Such other information may include, but is not limited to, information about the patient and/or information about patient support apparatus 20. Such patient information may include the patient's name; allergy information about the patient; medications the patient is on; diagnoses of the patient; historical treatment information regarding treatments, therapies, medications, etc. that have been previously performed, or administered to, the patient; any contents of medical records or medical history that is stored in EMR server 92, patient support apparatus server 84, and/or onboard patient support apparatus 20; the patient's date of birth, weight, height, and/or other personal information; the date the patient entered the healthcare facility; and/or still other information. The information about patient support apparatus 20 may include information relating to any one or more of the following: alarms or alerts of patient support apparatus 20; one or more settings of patient support apparatus 20; the armed/disarmed state of exit detection system 136 and/or monitoring system 138; a weight and/or weight history of the patient as determined from scale system 144; the room number or bay identifier in which patient support apparatus 20 is currently positioned, or other location information about patient support apparatus 20 (e.g. the wing, floor, department, department name, etc. of the healthcare facility); status information about one or more components of patient support apparatus 20; a log, or excerpts of, a log of equipment that has been added to, or removed from, an equipment weight log maintained by patient support apparatus 20 (which patient support apparatus 20 uses to distinguish patient weight from the weight of non-patient items on patient support apparatus 20); and/or other patient support apparatus information.

Another automatic action that controller 140 is configured to undertake in response to detecting a badge 142 within space volume 152c is, in some embodiments, to determinate what access to grant the person with the badge 142 to controls, menus, settings, etc. of patient support apparatus 20. For example, in some embodiments, if controller 140 determines that an associated badge 142 belongs to a service technician, controller 140 may only allow the service technician to access those menus, settings, etc. that relate to servicing patient support apparatus 20, and prevent that person from accessing menus, settings, therapies, etc. that are in place for the particular patient assigned to patient support apparatus 20. Similarly, if controller 140 determines that an associated badge 142 belongs to a caregiver, controller 140 may be configured to prevent the caregiver from accessing service data, configuration settings, diagnostic service, and/or other menus, screens, and/or settings that relate to maintaining the operation of patient support apparatus 20, but allow the caregiver to access controls, menus, screens, and/or settings that relate to caring for the patient.

As another example, if controller 140 determines that an associated badge belongs to an administrator or supervisor, controller 140 may automatically grant access to that person to menus, settings, and/or controls that caregivers and/or other classes of users are not allowed. In some embodiments, controller 140 allows administrators or supervisors to change settings on patient support apparatus 20 that are implemented or dictated by healthcare facility policies, but prevents other users of patient support apparatus 20 from changing those policy-based settings. Such policy settings may include, but are not limited to, whether or not monitoring system 138 needs to be armed (and/or in which situations; whether or not exit detection system 136 needs to be armed (and/or in what situations); whether reminders are issued or not issued by patient support apparatus 20; and/or other policy based settings of patient support apparatus 20.

FIGS. 10 and 11 illustrate one manner in which controller 140 may make automatic changes to the access levels granted to a user based upon the class of user who is wearing an associated badge 142. FIGS. 10 and 11 illustrate two different menu screens 170 and 170a, respectively, that controller 140 is adapted to display on display 52, depending upon the class of user to which an associated badge 142 belongs. For example, if a caregiver with a badge 142 moves into volume of space 152c (and therefore badge 142 becomes associated with patient support apparatus 20), controller 140 may be configured to display screen 170 on display 52. If a badge 142 associated with a technician becomes associated with patient support apparatus 20, controller 140 may be configured to display screen 170a on display 52.

In some embodiments, controller 140 displays menu screen 170 (FIG. 10) after a user presses on control 50f (FIG. 2), although menu screen 170 may be navigated to by the user in other manners. As noted, controller 140 may only display menu screen 170 if the user is wearing a badge 142 that corresponds to a particular class of users. Menu screen 170 includes an exit detection system control 50a, a monitoring system control 50b, a scale system control 50c, a lock control 503, a history control 50u, a night light control 50v, a settings control 50w, a service control 50x, a help control 50y, a pressure relief control 50z, a turn control 50aa, a pulmonary mattress control 50bb, a cancel icon 172, and a home icon 174.

Controls 50a, 50b, 50c, and 50e may, when pressed, undertake the same operations as controls 50a, 50b, 50c, and 50e, respectively, that were previously discussed with respect to FIG. 2. History control 50u, when pressed, may display a historical information regarding patient support apparatus 20, such as, but not limited to, software updates, patient weight histories, historical patient data, etc. Night light control 50v, when pressed, allows a user to turn on/off a night light on patient support apparatus 20, and/or configure the situations in which controller 140 will automatically turn on/off the night light. Settings control 50w, when pressed, takes the user to one or more settings screens that shows the current settings of patient support apparatus 20 and allows the user to change those settings. Service control 50x, when pressed, takes the user to one or more service screens that show a service history, current and/or past faults of patient support apparatus 20, and/or other service information. Help control 50y, when pressed, displays help information to a user that helps them understand the operation and usage of patient support apparatus 20. Pressure relief control 50z, turn control 50aa, and pulmonary control 50bb all control different functions and/or therapies that may be implemented using an inflatable mattress 42 positioned onboard patient support apparatus 20. Cancel icon 172, when pressed, causes controller 140 to stop displaying menu screen 170, and instead return to the previously displayed screen. Home icon 174, when pressed, causes controller 140 to display a home screen on display 52.

FIG. 11 displays one example of a modification to menu screen 170a that controller 140 may implement for one or more classes of healthcare workers (as determined from the IDs 166 of their badges 142). As shown therein, menu screen 170a only includes controls 50w and 50x and omits all of the other controls 50 that appear on menu screen 170. Menu screen 170a is intended to be displayed on patient support apparatus 20 when the badge 142 of a nearby user corresponds to a technician, service person, or the like. Menu screen 170a only allows that person to access the settings and service menus of patient support apparatus 20, which are the screens of most interest to such a person. Therefore, in at least one embodiments, controller 140 is configured to display screen 170a when an associated badge 142 corresponds to a service person, and to display screen 170 when an associated badge corresponds to a caregiver.

It will be understood that FIGS. 10 and 11 provide merely one example of the types of differences in access, menus, and/or screens that controller 140 may be configured to implement for different classes of users. Controller 140 may be configured to implement still other menu screens 170 for still different classes of user; it may configured to modify the accessible controls from what is shown in FIGS. 10 and 11; and/or it may be modified to undertake still other actions that automatically limit the accessibility, usability, and/or controllability of patient support apparatus 20 to different classes of users (as determined from their badges 142).

Another action that controller 140 is configured to automatically undertake in response to detecting an associated badge 142, in at least some embodiments, is to automatically record actions undertaken by a particular user that involve patient support apparatus 20. That is, in some embodiments, controller 140 is configured to record all, or a specified subset of, the functions that a person (with a badge 142) performs on patient support apparatus 20 while that person's badge 142 is associated with patient support apparatus 20. Thus, once controller 140 detects a badge 142 has entered volume of space 152c, it begins monitoring any functions of patient support apparatus 20 that may be carried out using any one or more components of patient support apparatus 20, records these functions (using a function identifier), records the unique ID 166 of the badge 142 of the person who was associated with patient support apparatus 20 at the time of those functions were carried out, and stores that information in memory 134 (and/or sends that information to patient support apparatus server 84). In some embodiments, controller 140 is also configured to record the current location of patient support apparatus 20 at the time these functions were implemented, the patient assigned to patient support apparatus 20 at the time these functions were implemented, and/or other information when these functions were implemented.

The automatic recording of patient support apparatus functions that is carried out by controller 140 may include any one or more of the following functions: changing a state of brake 300 on patient support apparatus 20; changing a height of litter frame 28; changing an angle of head section 44; making other changes to the angle, position, and/or configuration of litter frame 28 and/or support deck 30; raising or lowering siderails 36; arming or disarming exit detection system 136; arming or disarming monitoring system 138; weighing a patient using scale system 144; zeroing, or making other changes to, scale system 144; adding or removing items from an equipment weight log maintained by patient support apparatus 20; deleting patient data stored in memory 134; entering new patient information into memory 134; locking or unlocking any one or more controls of patient support apparatus 20; power cycling patient support apparatus 20; plugging in or unplugging AC cord 150 into or out of an electrical outlet 108; plugging in or unplugging nurse call cable 66 into or out of cable port 148 and/or communication outlet 64; moving patient support apparatus 20 to a different location (as detected through the connection/disconnection of patient support apparatus 20 from multiple locator units 60); adjusting the inflation state of mattress 42; implementing a therapy and/or a patient turn using inflatable mattress 42; changing a setting of patient support apparatus 20; and/or other changes.

Each of these functions is carried out by manipulating one or more controls onboard patient support apparatus 20, and controller 140 is configured to assign a function identifier to each of these functions. Further, controller 140 is configured to forward these function identifiers to patient support apparatus server 84 after these functions are carried out. Patient support apparatus server 84 has a directory of function identifiers and the functions corresponding to these identifiers, which it uses to determine from the function identifier what functions have been performed on patient support apparatus 20. Patient support apparatus server 84 may then forward information about the functions that have been performed to one or more recipients, such as EMR server 92, one or more electronic devices 98, one or more computers on network 80 (e.g. computer 168; FIG. 8), and/or to other recipients.

In some embodiments, controller 140 is configured to allow a user to customize what functions are automatically recorded and forwarded to patient support apparatus server 84. This customization may be carried out by an authorized user utilizing control panel 54c and/or by accessing patient support apparatus server 84. For example, controller 140 may be customized by an authorized user to not record any functions that are implemented on patient support apparatus 20 via the patient control panels 54c, but instead only record functions performed using caregiver controls panels 54a and/or 54c (and/or functions that don't require utilizing a control panel). As another example, controller 140 may be configured by an authorized user to not record when changes to the positions of siderails 36 are made, but record when changes to the brake 300, exit detection system 136, monitoring system 138 (and/or other changes) are made. Still other variations, of course, may be customized by authorized personnel.

Some functions that are completed by a user wearing a badge 142 may be desirably recorded in EMR server 92. For example, patient weight readings taken using scale system 144 may be desirable recorded in EMR server 92. Controller 140 is further customizable by the user as to which functions are to be recorded and automatically forwarded to EMR server 92. When controller 140 forwards these completed functions to EMR server 92, controller 140 also forwards caregiver identity information to EMR server 92 regarding the identity of the caregiver who implemented these functions. In some instances, the identity information includes the name and/or a unique identifier of the caregiver. Patient support apparatus server 84 determines the caregiver's name or unique identifier using the current location of patient support apparatus 20 and by receiving data from caregiver assignment server 104, which, as noted previously, may store the names of caregivers (or their unique IDs) and their corresponding assignments to specific rooms and/or bays.

Function data that is recorded on patient support apparatus 20 (e.g. in memory 134) and/or in patient support apparatus server 84 is subsequently retrievable by authorized personnel. Such data may be retrieved using a conventional computer, such as computer 168 (FIG. 8) that has access to network 80 and patient support apparatus server 84, by using an electronic device 98 that has access to network 80 and patient support apparatus server 84, and/or by one or more of the control panels 54 of patient support apparatus 20. By retrieving this information, authorized users can see not only the work performed by individual caregivers, but can also determine whether procedures, therapies, and/or treatments were completed in a proper and/or timely manner.

Another action that controller 140 is configured to automatically undertake in response to detecting an associated badge 142, in at least some embodiments, is to automatically validate certain functions that were performed by a particular user that involve patient support apparatus 20. That is, in some embodiments, controller 140 is configured to record when a caregiver's badge is associated with patient support apparatus 20 and to automatically validate any information the caregiver generates that is to be entered into EMR server 92. For example, in some EMR systems and/or in some healthcare facilities, a caregiver is required to validate certain types of information that is entered into an EMR server 92 during his/her shift. This typically requires the caregiver to manually access EMR server 92 and send information to it confirming that one or more treatments, protocols, medications, etc. that are recorded in the patient's electronic medical records were, in fact, performed or supervised by that particular caregiver. Controller 140 and server 84, in some embodiments, are configured to automatically take care of this validation process for at least some, if not all, of the information that caregivers are required to validate for a given EMR system and/or a given healthcare facility.

The automatic validation process carried out by controller 140 and patient support apparatus server 84 is similar to the automatic recordation of the various functions of patient support apparatus, except that controller 140 and/or server 84 send a validation message to EMR server 92, rather than, or in addition to, the data that is to be recorded in a particular patient's electronic medical record. For example, if a caregiver is required to perform rounding duties with each of his or her patients once every two hours, and the caregiver enters data into EMR server 92 indicating that these rounding duties were performed for a particular patient at a certain time, controller 140 and/or patient support apparatus server 84 are configured to validate that these rounding duties were, in fact, performed. This validation is carried out by sending data indicating that the specific badge 142 assigned to a particular caregiver was present (i.e. within space volume 152c) at a particular time, thereby confirming that the caregiver visited the patient at that particular time. In some embodiments, controller 140 and/or patient support apparatus server 84 are configured to both automatically record a caregiver's visit to a particular patient, and to validate that visit as well.

Another action that controller 140 is configured to automatically undertake in response to detecting an associated badge 142, in at least some embodiments, is to automatically log in the details of a caregiver's badge 142 into badge server 94. That is, as mentioned previously, healthcare workers often pick up a random badge 142 from a collection of badges 142 when they start their work shift. In order for badge server 94 to know which badge 142 belongs to which healthcare worker, the worker typically has to manually enter information into server 94, such as a badge ID, his/her name, etc. Patient support apparatus 20 and patient support apparatus server 84 are configured, in at least one embodiment, to automate this log in process. Controller 140 and patient support apparatus server 84 are configured to automate this process by monitoring which patient support apparatuses 20 a particular badge 142 travels to during a portion of the worker's shift, and then consulting caregiver assignment server 104 to determine which caregivers are assigned to which patients. For example, if a first badge 142 is moved into room 403a and detected by the patient support apparatus 20 in that room (which reports it to server 84), and then it moves to room 404 and is detected by the patient support apparatus 20 in that room (which also reports it to server 84), and then it moves into room 405 and is detected by the patient support apparatus 20 in that room (which also reports it to server 84), server 84 will know that that first badge 142 has traveled to rooms 403a, 404, and 405. From this information, patient support apparatus server 84 is configured to consult caregiver assignment server 104 to see which caregiver is assigned to the patients in rooms 403a, 404, and 405. Once server 84 determines the caregiver assigned to rooms 403a, 404, and 405, server 84 sends a message to badge server 94 indicating that the ID 166 of the first badge 142 belongs to a specific caregiver (e.g. caregiver X).

After badge server 94 receives this information from patient support apparatus server 84, badge server 94 thereafter associates the first badge 142 with that specific caregiver (e.g. caregiver X). In some embodiments, patient support apparatus server 84 may be configured to automatically send a message to the first badge 142 asking the caregiver to confirm his or her identity. Patient support apparatus server 84 undertakes this automated messaging in those systems where badges 142 are designed to allow the caregiver to press a button, or otherwise, take an action that confirms or disconfirms their identity, as determined by patient support apparatus server 84. In other words, patient support apparatus server 84 may be configured to send to badge 142 (via access points 82 and the WiFi transceiver 164 onboard badge 142) a message asking the caregiver: are you caregiver X (where X is an identity of one of the caregivers of that particular healthcare facility). The caregiver can then manipulate a control on the badge 142 that either affirms that that is in fact their identity, or that indicates that that is not their identity. After manipulating the control, badge 142 uses network transceiver 164 to transmit a message back to patient support apparatus server 84 indicating that their identity, as determined by patient support apparatus server 84, is either correct or incorrect.

It will be understood that, although the above-described example of the automated login process involved the caregiver visiting three different rooms before patient support apparatus server 84 determined the caregiver's identity, patient support apparatus server 84 may be configured to attempt to determine the caregiver's identity based on a single visit to a single patient support apparatus 20 (or other numbers of visits). After such a single visit, patient support apparatus server 84 consults caregiver assignment server 104 to see which caregiver is assigned to a particular room or patient, and then may send a message to badge 142 asking the caregiver to either approve or deny the caregiver identity that patient support apparatus server 84 has determined. If denied, patient support apparatus server 84 may send another message to that badge 142 with another proposed caregiver identity, either after the badge visits one or more additional patient support apparatuses 20, and/or before such additional visits are made. If approved, patient support apparatus server 84 sends a message to badge server 94 automatically logging that badge 142 and caregiver into the system so that badge server 94 knows the caregiver using that particular badge 142 for that particular work shift.

Controller 140 of patient support apparatus 20 may also be configured to automatically associate and disassociate patient support apparatus 20 from other devices 100 besides badges 142. Once such device 100 is depicted generically in FIGS. 4 and 5. Device 100 includes, at a minimum, a UWB transceiver 178 and a unique ID 180 (FIG. 5). Device 100 may also, depending upon the particular device 100, include a device controller 182 and/or one or more sensors 184. Device 100 may also include additional structures beyond those shown in FIG. 5. In some embodiments, UWB transceiver 178 and ID 180 are incorporated into a tag that is separable from, but attachable to, the device 100. Such UWB tags may take on any of the forms of the tags described in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which is incorporated herein by reference. In other embodiments, UWB transceiver 178 and unique ID 180 may be built-into the device 100 and not incorporated into a separable tag.

Controller 140 of patient support apparatus 20 automatically associates patient support apparatus 20 with, and disassociates patient support apparatus 20 from, one or more devices 100 by repetitively determining the relative location of UWB transceiver 178 of each of the various devices 100 with respect to patient support apparatus 20. As will be discussed in greater detail below, controller 140 may associate the device to patient support apparatus 20 if it moves inside of one of the space volumes 152 (also discussed in more detail below), and disassociate the device 100 if it moves outside of one of these space volumes 152. The types of devices 100 to which such tags may be attached, or into which a UWB transceiver 178 and ID 180 may be built in, include, but are not limited to, any one or more of the following: another patient support apparatus 20, an infusion pump, a vital sign sensor, an oxygen bottle, an exercise device, a heel care boot, an IV stand and/or pole, a ventilator, a DVT pumps, a patient monitor (e.g. a saturated oxygen (SpO₂) monitor, an EKG monitor, a vital sign monitor, etc.), a patient positioning devices (e.g. a wedge, turning device, pump), an ambient sensor (e.g. air temperature, air flow, light, humidity, pressure, altitude, sound/noise), a mattress 42, a portable exit detection sensor, an attachable nurse call device, an incontinence pad or one or more sensors adapted to detect patient incontinence, a Holter device adapted to monitor and record a patient's heart signals, a patient ID tag or bracelet worn by the patient that identifies the patient, a caregiver tag or ID bracelet worn by a caregiver that identifies the caregiver, a patient temperature management device (or associated device, such as a one or more hoses, thermal wraps, etc.), one or more mobility assistance devices that a patient may be expected to use, and/or still other types of devices 100.

In those embodiments where devices 100 include an infusion pump, patient support apparatus 20 and patient support apparatus server 84 may be configured to carry out any of the functions associated with the infusion pump that are described in commonly assigned U.S. patent application Ser. No. 63/349,369 filed Jun. 6, 2022, by inventors Krishna Bhimavarapu et al. and entitled COMMUNICATION SYSTEM FOR PATIENT SUPPORT APPARATUSES, the complete disclosure of which is incorporated herein by reference. In those embodiments where devices 100 include a portable exit detection sensor, another patient support apparatus, and/or an attachable nurse call device, patient support apparatus 20 and patient support apparatus server 84 may be configured to carry out any of the functions associated with the portable exit detection sensors, nurse call devices, and secondary patient support apparatuses disclosed in commonly assigned U.S. patent application Ser. No. 63/352,061 filed Jun. 14, 2022, by inventors Jerald Trepanier et al. and entitled COMMUNICATION SYSTEM FOR PATIENT SUPPORT APPARATUSES, the complete disclosure of which is incorporated herein by reference.

Controller 140 may be configured to associate a particular device 100 to patient support apparatus 20 via either a manual process or via an automatic process. For the manual process, the caregiver has to manually inform patient support apparatus 20 that a particular device 100 should now be associated with that patient support apparatus 20. This manual process may be accomplished in different manners. For example, in some embodiments, the device 100 and patient support apparatus 20 may include near field transceivers that, when positioned within close proximity (e.g. several inches) of each other, exchange information that establishes that that particular device 100 should be associated with that particular patient support apparatus. Further details regarding the use of near field transceivers for associating devices 100 to a patient support apparatus 20 are disclosed in commonly assigned U.S. patent application Ser. No. 63/352,061 filed Jun. 14, 2022, by inventors Jerald Trepanier et al. and entitled COMMUNICATION SYSTEM FOR PATIENT SUPPORT APPARATUSES, the complete disclosure of which has already been incorporated herein by reference. Other types of manual associations processes may also, or alternatively be used, some of which are also disclosed in the aforementioned '061 application.

Controller 140 is also configured to automatically associate at least some devices 100 with patient support apparatus 20 based upon the proximity of those devices 100 to patient support apparatus 20. This automatic association may be accomplished by the device 100 moving inside of a predetermined volume of space 152 defined with respect to patient support apparatus 20 (e.g. one of space volumes 152a-c, or another space volume; FIG. 6).

Controller 140 is also configured to automatically disassociate one or more devices 100 from patient support apparatus 20. The automatic disassociation may be accomplished by the device 100 moving outside of the same predetermined volume of space 152. Alternatively, controller 140 may use modified volumes of space-such as, but not limited to, larger space volumes-when automatically determining whether to disassociate one of these devices 100 from patient support apparatus 20. In other words, once a device 100 has been determined to be positioned inside of a particular volume of space, such as space volumes 152a-c (and any additional association conditions are met, if there are any), and controller 140 has associated the device 100 with patient support apparatus 20, controller 140 may thereafter increase the size of—and/or otherwise change one or more dimensions of—the volume of space when determining whether to disassociate the device 100. In this manner, the volumes of space 152 may have a sort of hysteresis aspect wherein a device 100 has to be positioned inside of a smaller space volume in order to be associated with patient support apparatus 20, but thereafter can only be disassociated if it moves outside of a larger sized volume of space. In still other embodiments, the dimensions of one or more of the volumes of space are the same for both association and disassociation purposes.

Once a device 100 is associated with patient support apparatus 20, it thereafter remains associated with patient support apparatus 20 until it moves outside of a volume of space, such as space 152b of FIGS. 4 & 6, at which point controller 140 disassociates the device 100 from patient support apparatus 20. Controller 140 repetitively determines and monitors the position of device 100 while it is within communication range of controller 140. Specifically, controller 140 uses UWB transceivers 132, which communicate with UWB transceiver 178 onboard device 100, to repetitively determine the relative position of device 100 with respect to patient support apparatus 20. Controller 140 compares this relative position to a particular space volume 152 and, if it is outside of the space volume 152, it automatically disassociates device 100 from patient support apparatus 20. On the other hand, as long as the position of device 100 remains inside of the space volume 152, controller 140 maintains the association of device 100 with patient support apparatus 20. As noted previously, the dimensions of the space volume 152 that are used for the association/dissociation process of device 100 may be the same as, or different from, the dimensions of space volume 152 that are used for the association/dissociation process.

In some embodiments, space volumes 152a-c (FIGS. 4 & 6) is defined with respect to patient support apparatus 20 and therefore move as patient support apparatus 20 moves. Space volume 152a, which may be the smallest of the space volumes 152, is generally used for the automatic association and disassociation between patient support apparatus 20 and a locator unit 60. Space volume 152b is generally used for the automatic association and disassociation between patient support apparatus 20 and a variety of different devices 100. Space volume 152c is generally used by controller 140 for the automatic association and disassociation between patient support apparatus 20 and badges 142. It will be understood that controller 140 may utilize other space volumes 152 than the three shown in FIGS. 4 and 6.

Space volume 152c is generally sized such that it encompasses substantially all of the room 58 in which patient support apparatus 20 is positioned, or at least that portion of the room in which a healthcare worker with a badge 142 is expected to be present, particularly while the worker attends to the patient assigned to patient support apparatus 20 and/or to patient support apparatus 20 itself. In some situations, space volume 152c may be large enough to extend into adjacent rooms 58, but this will not affect the proper association of a badge 142 with patient support apparatus 20 so long as access to those rooms cannot be obtained without the healthcare worker exiting from space volume 152b before traveling to those other rooms. In other words, space volume 152c may be advantageously defined such that, when the person's badge 142 is moved out of room 58 through a doorway, the badge 142 will necessarily move outside of space volume 152c, thereby causing it to become disassociated from the patient support apparatus 20.

In some embodiments, any of space volumes 152a-c may be defined with one or more static dimensions. In other embodiments, patient support apparatus 20 and/or patient support apparatus server 84 may be configured to allow authorized individuals to change one or more dimensions of space volumes 152a-c. Still further, in some embodiments, space volume 152a-c may have variable dimensions based upon the specific room, bay, or other location, in which patient support apparatus is currently positioned. In these embodiments, controller 140 may utilize a table stored in memory 134 that defines the dimensions of any of space volumes 152a-c based on the current location of patient support apparatus 20. The location of patient support apparatus 20 may be determined by controller 140 from the locator unit ID 122 that it receives from an associated locator unit 60, and/or it may be derived from information received from patient support apparatus server 84.

Volume of space 152c may be different from volume of space 152a (and, as will be discussed in greater detail below, volume of space 152b). FIG. 6 illustrates an example of an arbitrary healthcare facility room 58 in which is positioned a patient support apparatus 20, a device 100, and a badge 142. FIG. 6 also illustrates first volume of space 152a, second volume of space 152b, and third volume of space 152c. First volume of space 152a, as has been discussed, is used to determine whether patient support apparatus 20 should associate or disassociate itself from the locator unit 60 that is positioned adjacent to, or inside of, first volume of space 152a. Second volume of space 152b is used by controller 140, as has been noted, to determine whether to associate or disassociate a device 100 with patient support apparatus 20. Third volume of space 152c may be used by controller 140 to determine whether to associate or disassociate one or more badges 142 with patient support apparatus 20. Second volume of space 152b is generally smaller than third volume of space 152c because devices 100 are typically placed in close proximity to patient support apparatus 20 when used in the care of the patient (as opposed to a badge 142, which may be positioned further away from patient support apparatus 20, such as when a healthcare walks about the interior or a room). It can therefore be seen that controller 140 of patient support apparatus 20 may be configured to use multiple different volumes of space 152a-c for determining whether to associate a device 100 with patient support apparatus 20—one for locator units 60, another for devices 100, and still another for badges 142. Still other space volumes for different types of devices 100 may also be used.

As shown in FIG. 6, first volume of space 152a is dimensioned so that it will encompass fixed locator unit 60 when patient support apparatus 20 is positioned within about 5 to 10 feet of a locator unit 60. Second volume of space 152b encompasses patient support apparatus 20 and a relatively small amount of space surrounding patient support apparatus 20. This small amount of space around the perimeter of patient support apparatus 20 may be defined with the goal of encompassing IV poles, infusion pumps, or other medical devices that may be used in the care of the patient, but that may not necessarily be directly supported on patient support apparatus 20. Second volume of space 152b may also be defined such that it is unlikely to encompass devices 100 that are being used on a patient assigned to another patient support apparatus 20 that may be positioned nearby. Third volume of space 152c encompasses a substantial portion of room 58. It will, of course, be understood that the sizes and dimensions of volumes of space 152a-c may vary from what is shown in the attached drawings.

As shown in FIG. 6, a fourth volume of space 152d may also be defined for locator units, such as unlinked locator units 60a, that are positioned in hallways, and/or in other areas outside of patient rooms 58. Space volumes 152d may be sized to provide more generalized locations of patient support apparatuses 20 (and the devices associated therewith), such as being located in a particular hallway, or portion of a hallway, a particular storage area, etc. In some embodiments, space volume 152d is used by controller 140 when determining whether to associated with an unlinked locator unit 60a. In some embodiments, the dimensions of space volumes 152d for each unlinked locator unit 60a are stored within a memory of each unlinked locator unit 60a and transmitted to patient support apparatus 20 when patient support apparatus 20 is within communication range. Alternatively, the dimensions of space volume 152d may be stored onboard patient support apparatus 20 and consulted by controller 140 based upon the ID 122 received from an unlinked locator unit 60.

It will be understood that the dimensions of space volume 152d, like those of space volumes 152a, may be variable, customizable, location dependent, and/or different from what is shown in FIG. 6. It will also be understood that, although the accompanying drawings all depict all of the space volumes 152a-d as having rectangular shapes, these shapes may be varied, including shapes that are all curved and/or shapes that have a combination of curved and straight boundaries. It will also be understood that the volumes of space 152a-c may be alternatively referred to as threshold distances from patient support apparatus 20. If the threshold distance has a constant value in all directions from a measurement point of the patient support apparatus 20, the volume of space 152 will define a sphere. If the threshold distance has a non-constant value in one or more different directions from the measurement point of the patient support apparatus 20, the volume of space 152 will define a non-spherical shape.

In some embodiments, one or more volumes of space 152 may be defined that are large enough to encompass areas outside of a healthcare facility room, such as in the hallway(s) and/or in storage areas. In some embodiments, a separate volume of space 152 may be defined that encompasses one or more storage areas (e.g. closets, cabinets, etc.) associated with a particular room and another additional volume of space 152 may be defined that encompasses areas of an adjacent hallway. As will be discussed in greater detail below, the volume of space that encompasses one or more storage areas may be used by patient support apparatus 20 and/or server 84 to implement an inventory management system that informs appropriate personnel (via badges 142 and/or electronic devices 98) when items in the storage area need replacing. As will also be discussed in greater detail below, the volume(s) of space that encompass the hallway(s) outside of a room may be used by patient support apparatus 20 to detect nearby caregiver badges 142 and/or electronic devices 98 so that notifications can be sent to those devices when patient support apparatus 20 detects one or more conditions that are in an undesired state.

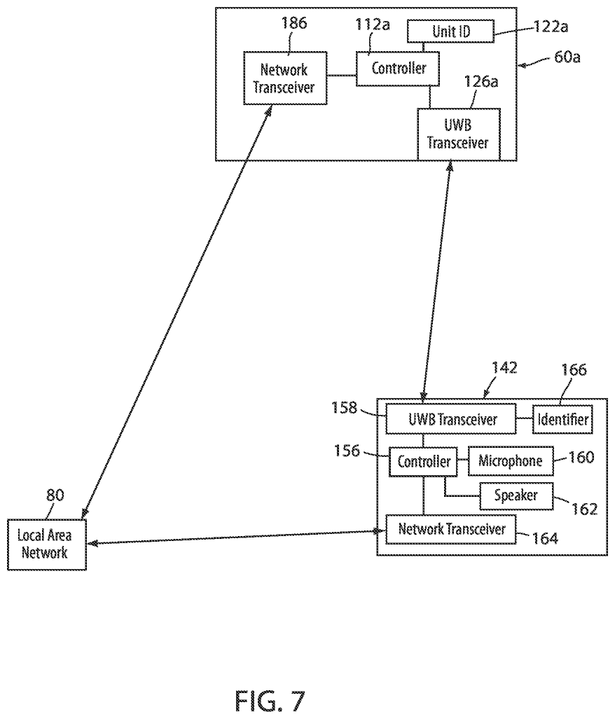

FIG. 7 illustrates an example of the internal components of an unlinked locator unit 60a (as well as a badge 142 and local area network 80). Unlinked locator unit 60a includes a number of components that are the same as those found in linked locator units 60. Those common components are labeled with the same reference number followed by the letter “a.” Components of unlinked locator units 60a that are not found in linked locator units 60 are provided with a new reference number.

Unlinked locator unit 60a includes a controller 112a, a UWB transceiver 126a, a unit ID 122a, and a network transceiver 186. Network transceiver 186 is, in at least one embodiment, a WiFi transceiver adapted to be able to communicate with network 80 via wireless access points 82. Controller 112a is adapted to determine the location of badges 142 that are positioned within range of UWB transceiver 126a. In some embodiments, unlinked locator unit 60a includes multiple UWB transceivers 126a, while in other embodiments, unlinked locator unit 60a may include only a single UWB transceiver 126a. Regardless of how many UWB transceivers 126a unlinked locator unit 60a includes, controller 112a is configured to use UWB transceiver(s) 126a to measure the distance between itself and any badges 142 that are positioned within range of unlinked locator unit 60a. In some situations, such as where unlinked locator unit 60a includes only a single UWB transceiver 126a, controller 112a may only be able to determine a distance between itself and the badge 142, rather than a three-dimensional position. In other situations, such as where unlinked locator unit 60a includes multiple UWB transceivers 126a, controller 112a may be able to determine a three-dimensional position of a badge 142 relative to unlinked locator unit 60a.

Regardless of the 1-dimensional, 2-dimensional, or 3-dimensional position determination of a badge 142, controller 112a is configured to send a message to patient support apparatus server 84 via network transceiver 186 that identifies the detected location of the badge 142 with respect to unlinked locator unit 60a. This message also identifies the unique identifier 122a of the unlinked locator unit 60a. Patient support apparatus server 84 is therefore able to determine the general location of the badge 142 within the healthcare facility using the stored positions of each locator unit 60 within the healthcare facility.

In addition to tracking the position of badges 142 as they move through the healthcare facility, unlinked locator units 60a may also be provided within a healthcare facility in order to provide location information to patient support apparatuses 20 as they are moved outside of the rooms within the healthcare facility. Patient support apparatus 20, in some embodiments, is adapted to communicate with UWB transceiver 126a of unlinked locator units 60a in the same manner that it communicates with UWB transceiver 126 of linked locator units 60. This enables patient support apparatus 20 to determine its relative position with respect to the unlinked locator unit 60a (which, in some embodiments, is a three-dimensional position). Controller 140 can therefore determine its position within the healthcare facility, and/or forward the locator ID 122a from the unlinked locator unit 60a to patient support apparatus server 84, and patient support apparatus server 84 can utilize this information to determine the current location of patient support apparatus 20 within the healthcare facility.

Controller 140, in some embodiments, is configured to automatically send a notification message to the nearest badge 142 notifying it of undesired states, such as, but not limited to, error states, of patient support apparatus 20 and/or error states of any devices 100 with which patient support apparatus 20 is in communication. For example, if a patient support apparatus 20 is operating on battery power and the battery level falls below a particular threshold, controller 140 is configured to send a notification message to patient support apparatus server 84 informing server 84 of the low battery condition. As will be described more below, patient support apparatus 20 may also send notification messages when one or more inventory items need restocking, when patient support apparatus 20 needs to be cleaned, or should be marked as already having been cleaned, when an oxygen bottle needs to be replaced or refilled, when fluid for an infusion pump is running low, when equipment should be added to patient support apparatus 20, when equipment can be removed from patient support apparatus 20, and/or when other conditions with undesirable states are detected.

When controller 140 sends a notification message, controller 140 may be configured to send the message directly to a nearby badge 142 and/or it may send the notification message to patient support apparatus server 84, which is configured to check the locations of all of the badges 142 and determine which one is closest to the patient support apparatus 20 with the undesired condition (e.g. low battery). Patient support apparatus 20 may then automatically send a message to that badge 142, via wireless access points 82, informing the caregiver that the patient support apparatus 20 in room such-and-such needs its battery re-charged and/or needs to be plugged into a power outlet. Controller 140 may send the notification message to badge 142 directly using one or more of the UWB transceivers 132; it may send the notification message directly to badge 142 using a Bluetooth transceiver 128; and/or it may send the notification message indirectly by first sending the message to patient support apparatus server 84 or badge server 94 (using network transceiver 96), and then having that server forward the notification message to the badge via the badge's network transceiver 164.

When carrying out the automatic notification process, controller 140 may be configured to first check to see if any badges 142 are currently positioned within volume space 152c before sending the notification message to server 84. If a badge 142 is positioned therein, controller 140 may send a notification message to that particular badge 142 that doesn't need to pass through server 84. The message informs the caregiver associated with that badge of the low battery condition, or other undesired state.

In some embodiments, patient support apparatus server 84 may be configured to not send a notification message to any badges 142 of the undesired state of a patient support apparatus 20 unless the badge 142 is positioned within a set threshold of the patient support apparatus 20. In other words, in some embodiments, patient support apparatus server 84 is configured to only notify healthcare workers who happen to walk by, or near, the patient support apparatus 20 that has the undesired condition. Thus, patient support apparatus server 84 may be configured to continuously monitor the location of badges 142 and, if none are currently positioned with the threshold distance, wait until one such badge 142 is positioned therein, and then send the notification message to the badge 142 informing the associated user of the undesired state of the patient support apparatus 20.

Patient support apparatus server 84 may also be configured to automatically rate the severity of the undesired state of the patient support apparatus 20 and use that rating to determine the size of the threshold distance, and/or to use that rating to determine whether or not to send a notification message to the badge 142 of the caregiver assigned to the patient in that particular patient support apparatus 20, regardless of the distance of that particular caregiver to the patient support apparatus 20 in the undesired condition. Thus, depending upon the severity of the undesired state of the patient support apparatus 20, a worker with a badge 142 may get notified of the undesired state immediately, regardless of their location, at a later time when they are at a medium distance to the patient support apparatus 20, or at another time when they are relatively close to the patient support apparatus 20. The distance threshold utilized by patient support apparatus server 84 may also take on other gradations besides “medium” and “close.”

It will be understood that the list of undesired states of patient support apparatus 20 that a nearby badge-wearing worker may be notified of can vary greatly. In some embodiments, controller 140 and patient support apparatus server 84 are configured to notify a nearby badge 142 of any one or more of the following undesired states: a siderail 36 of patient support apparatus 20 is not in a desired position; a power cord of patient support apparatus 20 is unplugged; a nurse call cable 66 is not plugged into patient support apparatus 20 and/or a linked locator unit 60; a charge level of battery 304 has dropped below a threshold; brake 300 of patient support apparatus 20 has been deactivated; a litter frame 28 has been moved to an undesired height; scale system 144 needs to be zeroed; and/or other states.

As an alternative to, or in addition to, sending a notification message to a badge 142, patient support apparatus 20 and/or patient support apparatus server 84 may be configured to send a notification message to an electronic device 98 of the caregiver who is associated with the nearby badge 142. In other words, instead of notifying a caregiver of an undesired state of patient support apparatus 20 via badge 142, patient support apparatus 20 and server 84 may notify the caregiver via his or her electronic device 84. In order to send a notification message to the caregiver's electronic device 98, server 84 consults another server on network 80, or a database accessible thereto, that associates healthcare workers with specific electronic devices 98. Server 84 uses that information to determine which specific electronic device 98 the notification message should be sent to, and then forwards the message using access points 82 (or, instructs patient support apparatus 20 which specific electronic device 98 to forward the notification message to, and controller 140 then sends the notification message to that specific electronic device 98 using one of its onboard transceivers).

It will also be understood that patient support apparatus server 84, when determining which badge 142 is nearest to a patient support apparatus 20 that is currently in an undesired state, may utilize the position information of badges 142 as determined by their UWB communications with locator units 60 and/or with patient support apparatuses 20, and/or it may utilize the position information of badges 142 as determined by badge server 94 using conventional WiFi location methods (e.g. triangulation, signal strength measurements, etc.). Thus, patient support apparatus server 84 is configured, in at least some embodiments, to determine the location of badges 142 using two independent location-determination systems: a UWB-based location system and a WiFi-based location determination system. The locations determined from both of these systems may be combined by patient support apparatus server 84 to determine the location of badges 142 with better accuracy than when using only a single one of these location systems.

In addition to, or in lieu of, sending messages to the badge 142 that is nearest to a patient support apparatus 20 that is currently detecting an undesired state, controller 140 may be configured to send messages and/or reminders to the badge 142 of the specific caregiver(s) assigned to the patient in the patient support apparatus 20 that is in the undesired state. In other words, rather than the nearest caregiver, controller 140 and server 84 may be configured to send a message to the badge 142 of the caregiver responsible for the patient support apparatus 20 that is currently in an undesired state. Controller 140 and/or server 84 may be configured to send these messages, or reminders, immediately upon detecting that a badge 142 has become disassociated from a patient support apparatus 20 that is in an undesired state. If the patient support apparatus 20 thereafter remains in an undesired state for more than a threshold amount of time, controller 140 and/or patient support apparatus server 84 may send one or more additional follow-up message to the badge 142 of the caregiver assigned to the patient in that particular patient support apparatus 20. In this manner, caregivers are automatically informed of any undesired states of any patient support apparatuses 20 that are being used with the patients they are assigned to care for.

In some embodiments, patient support apparatus 20 is configured to detect an undesired condition relating to the amount of inventory currently maintained in a nearby storage area, such as, but not limited to, a closet 316 (FIG. 4). That is, controller 140 is configured to communicate with one or more inventory devices 100 that have UWB tags attached to them (or UWB transceivers otherwise built into them). The inventory devices 100 may refer to any items that a healthcare facility likes to store and/or keep in stock within a closet, or other storage area, such as, but not limited to, linens, bedding, medicines, first aid items, any disposables, and/or still other items. Controller 140 uses UWB transceivers 132 to communicate with such inventory devices 100, to determine their relative position to patient support apparatus 20 (in order to determine if they are currently located in a storage area or not), and to count the number of such inventory devices 100 that it detects within the defined storage area. After counting the number of inventory devices 100 that are positioned within the defined storage area (e.g. closet 316), controller 140 then determines if one or more of the inventory devices 100 needs to be restocked.

In some embodiments, the determination as to whether any inventory device 100 needs to be restocked in a storage area is made by patient support apparatus server 84 working in conjunction with patient support apparatus 20 (and, in some cases, an inventory management system). In some embodiments, controller 140 may make this determination on its own. In either case, a desired or target number of inventory devices 100 are input into patient support apparatus 20 and/or into patient support apparatus server 84. In addition, the location of storage areas within a healthcare facility relative to locator units 60 is stored onboard patient support apparatus 20 and/or in server 84. Using this information, as well as patient support apparatus 20's relative position/orientation to a nearby locator unit 60, controller 140 is able to determine which devices 100 are positioned inside of a storage area and which devices are not positioned inside of the storage area. From the identifiers 180 received from the devices positioned inside of the storage area, controller 140 is able to count the number of particular devices 100 that are currently positioned inside of the storage area.

After the total number of one or more types of devices 100 are determined to be within a storage area, controller 140 either compares the counted number to a target or desired number for that particular type of device 100, or forwards the counted number to server 84 and server 84 compares the counted number to the target number. If the counted number of devices is less than the target number, controller 140 and/or server 84 issues a notification. The notification may take on different forms, but is intended to inform appropriate personnel that one or more devices 100 should be restocked in the particular storage area where their counted number was determined to be less than their desired number. In some embodiments, the notification includes displaying information on display 52 of patient support apparatus 20, transmitting a message to server 84 that it then forwards to one or more electronic devices 98 and/or badges 142, sending a message directly to one or more badges 142 using one or more of UWB transceivers 132 and/or another transceiver onboard patient support apparatus 20, and/or sending a message to a restocking unit 320 positioned near the storage area, and/or taking other actions to notify appropriate personnel of the need to restock one or more items.

Data defining the particular locations within a healthcare facility where storage areas are located is input into server 84 and/or controller 140. Controller 140 then uses this information to determine whether a restocking device 100 is positioned within a particular storage area. That is, controller 140 first determines the position and/or orientation of patient support apparatus 20 relative to a nearby locator unit 60. In some embodiments, controller 140 is configured to determine the position and orientation of patient support apparatus 20 relative to a locator unit 60 in any of the manners disclosed in commonly assigned PCT application serial number PCT/US2022/043585 filed Sep. 15, 2022, by Stryker Corporation and entitled SYSTEM FOR LOCATING PATIENT SUPPORT APPARATUSES, the complete disclosure of which is incorporated herein by reference. Other manners for determining the position and orientation of patient support apparatus 20 to a locator unit 60 may also, or alternatively, be used.

After determining its position and orientation relative to a particular locator unit 60, controller 140 consults memory 134 (or receives from server 84) the relative position of the storage area (e.g. closet 316) relative to that particular locator unit 60. From this information, controller 140 is able to determine is position and orientation with respect to closet 316, and therefore is able to use its UWB transceivers 132 to communicate with any devices 100 that are positioned within closet 316. As noted, controller 140 uses the IDs 180 from these devices to determine what type of devices they are, and then consults the desired number of those devices 100 that should be in stock for that particular storage area (or passes this information to server 84 to determine if restocking needs to take place).

It will be understood that the terms “device 100” and “inventory device 100” are broad terms that, as noted previously, may refer to a wide variety of different items that are used in a healthcare facility, including consumable items. It will also be understood that the different between a “device 100” and an “inventory device 100” is merely the fact that an “inventory device 100” refers to a device that the healthcare facility wishes to keep in stock at a certain number (and either patient support apparatus 20 or server 84 knows, or has access to, this number).

In some embodiments, controller 140 is configured to communicate with a restocking unit 320 (FIG. 4), either in addition to, or in lieu of, the inventory devices 100 that may be positioned inside of a storage area. Restocking unit 320 is a device that is adapted to keep track of device 100 as they are removed from the storage area and restocked within the storage area. In other words, restocking unit 320 is a device that is adapted to keep track of the inventory levels of the various devices 100 positioned within the storage area. In some embodiments, restocking unit 320 is adapted to keep track of items that are stocked in the storage area 316, regardless of whether or not such items have a UWB tag or UWB transceiver. Restocking unit 320 is further configured to communicate with patient support apparatus 20, such as via UWB transceiver(s) 132, Bluetooth transceiver 128, and/or network transceiver 96. Restocking unit 320 is thus able to forward to patient support apparatus 20 the inventory levels of each item stored in storage area 316 and/or the type and number of items that need restocking. By forwarding this information directly to patient support apparatus 20, neither controller 140 nor server 84 need to determine what items need to be restocked. Instead, controller 140 receives this information directly from restocking unit 320. In response this receipt, controller 140 issues one or more of the notifications previously discussed.

Restocking unit 320 may include one or more UWB transceivers that automatically detect when inventory devices 100 are added to, or removed from, storage area 316. Alternatively, or additionally, restocking unit 320 may include a bar code scanner, QR code scanner, near field reader, RFID tag reader, and/or other types of sensors that are adapted to detect/read corresponding codes, near field communicators, RFIDs, and/or other data that is coupled to the items stored in storage area 316. As these items are added to, or removed from, storage area, the caregiver passes the item close to the sensor/reader of restocking unit 320 and informs restocking unit 320 where the items are being added to, or removed from, storage area 316. In this manner, restocking unit 320 is able to keep track of the total number of each type of item stored in storage area 316. In some embodiments, restocking unit 320 may have a keypad, touchscreen, and/or other data entry device that allows the user to manually input information about what items are being added to, or removed from, storage area 316.

In some embodiments of restocking unit 320, restocking unit includes a light, a display, and/or a sound emitter that is adapted to alert nearby personnel when any one or more items need to be restocked in the adjacent storage area. Further, in some embodiments, controller 140 of patient support apparatus 20 may be configured to transmit a message to restocking unit 320 instructing it to flash a light, emit a sound, and/or display information on its display regarding items that need to be restocked. As was mentioned previously, restocking unit 320 is an optional item that patient support apparatus 20 may interact with, but it is not required for patient support apparatus 20 to be able to keep track of what items are currently in need of restocking within a nearby storage area 316.

In some embodiments, device 100 may be an infusion pump and controller 140 may be adapted to communicate with the infusion pump using UWB transceivers 132 to determine whether the infusion pump is being used with the patient assigned to that particular patient support apparatus 20. In other words, controller 140 may be configured to determine whether to associate the infusion pump 100 with patient support apparatus 20 or not. In such embodiments, controller 140 is adapted to monitor the amount of fluid remaining in the bag, or other reservoir, that provides fluid to the infusion pump. If the fluid level in the bag goes below a threshold (e.g. nearing empty), controller 140 is adapted to issue a notification. The notification may be issued in any of the manners previously discussed above. For example, the notification may involve controller 140 doing any one or more of the following: displaying information on display 52 of patient support apparatus 20, transmitting a message to server 84 that it then forwards to one or more electronic devices 98 and/or badges 142, sending a message directly to one or more badges 142 using one or more of UWB transceivers 132 and/or another transceiver onboard patient support apparatus 20, and/or sending a message to the infusion pump 100 itself, and/or taking other actions to notify appropriate personnel of the need to restock one or more items.

In some embodiments, device 100 may be an oxygen bottle and controller 140 may be adapted to communicate with the oxygen bottle using UWB transceivers 132 to determine whether the oxygen bottle is being used with the patient assigned to that particular patient support apparatus 20. In other words, controller 140 may be configured to determine whether to associate the oxygen bottle 100 with patient support apparatus 20 or not. In such embodiments, controller 140 is adapted to monitor the amount of oxygen remaining in the bottle. If the oxygen level in the bottle goes below a threshold (e.g. nearing empty), controller 140 is adapted to issue a notification. The notification may be issued in any of the manners previously discussed above (e.g. displaying information on display 52 of patient support apparatus 20, transmitting a message to server 84 that it then forwards to one or more electronic devices 98 and/or badges 142, sending a message directly to one or more badges 142 using one or more of UWB transceivers 132 and/or another transceiver onboard patient support apparatus 20, and/or sending a message to the oxygen bottle 100 itself, and/or taking other actions to notify appropriate personnel of the need to restock one or more items).

In some embodiments, controller 140 is adapted to determine whether patient support apparatus 20 needs to be cleaned and/or has been cleaned. In such embodiments, controller 140 may be configured to make such determinations in any of the manners disclosed in commonly assigned U.S. provisional patent application Ser. No. 63/357,363 filed Jun. 30, 2022, by inventors Madhu Sandeep Thota et al. and entitled BADGE AND PATIENT SUPPORT APPARATUS COMMUNICATION SYSTEM, the complete disclosure of which has already been incorporated herein by reference. When controller 140 determines that patient support apparatus 20 needs to be cleaned, or already has been cleaned, controller 140 is configured to remind one or more appropriate personnel that the status of patient support apparatus 20 (i.e. clean or needs cleaning) needs to be updated (such as in one or more servers on network 80). In such embodiments, controller 140 may be configured to issue a notification in any of the manners discussed above. The notification reminds the one or more recipients of the needs to update the cleanliness status of patient support apparatus 20. In some such embodiments, controller 140 may be configured to cancel or skip the notification if the server that keeps track of the cleanliness of patient support apparatus 20 has already been updated. In such embodiments, server 84 may query that server to determine the latest cleanliness status of patient support apparatus 20 and relay that information to controller 140. If that latest cleanliness status matches the status determined by controller 140, controller 140 does not issue a notification. If the latest cleanliness status does not match the status determined by controller 140, controller 140 issues the notification. The notification may be issued in any of the manners previously discussed above.

In some embodiments, controller 140 is adapted to detect what devices 100 are positioned within one or more volumes of space 152 and use that information to determine if any additional devices 100 are needed for the treatment of the particular patient assigned to patient support apparatus 20, or if any of the detected devices 100 are not actually necessary for the treatment of that particular patient. In such embodiments, controller 140 is configured to receive information, via patient support apparatus server 84, from electronic medical records (EMR) server 92 indicating the diagnosis and/or treatment protocols that have been determined for the particular patient. EMR server 92 may also include data indicating what devices 100 are to be used for treating that particular patient. Alternatively, or additionally, patient support apparatus server 84 and/or controller 140 may be configured to determine from the data contained within EMR server 92 what device(s) 100 are needed for treating the particular patient. In either case, controller 140 uses this information to determine if any devices 100 that are currently associated with patient support apparatus 20 are needed or unneeded for the treatment of that patient. Similarly, controller 140 determines if there any needed devices for treating the particular patient that are not currently associated with patient support apparatus 20. In either case (needed device(s) 100 are missing, or unneeded device 100(s) are present), controller 140 is adapted to issue a notification. The notification may be carried out in any of the manners previously discussed. The notification informs appropriate personnel that they may need to add needed equipment to patient support apparatus 20 and/or return unneeded equipment to another area of the healthcare personnel. In some embodiments, controller 140 is adapted to perform the check for needed/unneeded equipment whenever scale system 144 is about to be zeroed. In such embodiments, and/or in other embodiments, controller 140 is configured to check for needed/unneeded equipment (e.g. devices 100) at other times and/or repetitively.

In some embodiments, controller 140 is configured to monitor the charge status of battery 304 and share this information with nearby badges 142 of appropriate personnel in certain situations. In such embodiments, controller 140 may be configured to share the charge status of battery 304 both when battery 304 is nearly fully charged and when battery 304 is nearly fully drained. Controller 140 may also be configured to share the charge status of battery 304 when battery 304 is at a medium charge, or at any charge. In some situations, such as when battery 304 is fully charged, or has a charge level above a threshold, the purpose of controller 140 sharing the status of battery 304 is to inform nearby caregivers and/or other personnel that battery 304 is available for using on another patient support apparatus 20 should that other patient support apparatus 20 have a low battery. In other situations, such as where battery 304 is nearly drained, or has a charge level below a threshold, the purpose of controller 140 sharing the status of battery 304 is to inform nearby caregivers and/or other personnel that battery 304 should be recharged or replaced. Controller 140 can therefore be configured to notify personnel of the availability of battery 304 for use on another patient support apparatus and/or the need to recharge battery 304.

When battery 304 is above a certain charge threshold, controller 140 may be configured to automatically share the status of battery 304 with any badges 142 or UWB-equipped electronic devices 98 that are within a volume of space 152, such as a volume of space 152 that encompasses the hallway outside of the room in which patient support apparatus 20 may currently be positioned. In this manner, a caregiver, or other healthcare worker, who walks by the room in which patient support apparatus 20 is positioned will be notified via their badge 142 and/or UWB-equipped electronic device 98 of the nearly-fully charged battery 304 on patient support apparatus 20. The notification may be sent directly from patient support apparatus 20 to the badge 142 and/or electronic device 98 using one or UWB transceivers 132 or Bluetooth transceiver 128. Alternatively, the notification may be sent via network transceiver 96 to server 84, which may then forward the message to the badge 142 and/or electronic device 98 using one or more wireless access points 82. Controller 140 is adapted to automatically detect the presence of the badge 142 and/or electronic device 98 within the volume of space 152 in the manner previously described.

In some embodiments, when battery 304 is above a charge threshold, controller is configured to not share the status of battery 304 with any badges 142 and/or electronic devices 98 if the battery 304 is currently in use on patient support apparatus 20. In such embodiments, controller 140 is configured to only share the fact that its battery 304 is nearly fully charged when it is not currently using battery 304.

When battery 304 is below a certain charge threshold, controller 140 may be configured to automatically share the status of battery 304 with any badges 142 or UWB-equipped electronic devices 98 that are within a volume of space 152, such as a volume of space 152 that encompasses the hallway outside of the room in which patient support apparatus 20 may currently be positioned. In this manner, a caregiver, or other healthcare worker, who walks by the room in which patient support apparatus 20 is positioned will be notified via their badge 142 and/or UWB-equipped electronic device 98 of the nearly-drained battery 304 on patient support apparatus 20. The notification may be sent directly from patient support apparatus 20 to the badge 142 and/or electronic device 98 using one or UWB transceivers 132 or Bluetooth transceiver 128. Alternatively, the notification may be sent via network transceiver 96 to server 84, which may then forward the message to the badge 142 and/or electronic device 98 using one or more wireless access points 82. Controller 140 is adapted to automatically detect the presence of the badge 142 and/or electronic device 98 within the volume of space 152 in the manner previously described.

In some embodiments, patient support apparatus 20 is configured to automatically detect whether it is positioned within the vicinity of a charging station 308 (FIG. 12). In such embodiments, charging station 308 includes a UWB transceiver (not shown) that communicates with the UWB transceivers 132 onboard patient support apparatus 20. Controller 140 uses the UWB transceivers in the manner previously discussed to determine if charging station 308 is within a threshold distance or (or within volume of space 152 defined around) patient support apparatus 20. In some embodiments, the threshold distance is approximately equal to a length of a cord 314 of the charging station 308.

Charging station 308 is adapted to provide electrical power to recharge one or more batteries 304 onboard patient support apparatus 20. Charging station 308 includes a cable 314 having a plug 312 at one end that is adapted to attach or couple to a port, or other connector (not shown), on patient support apparatus 20. When plug 312 is attached to patient support apparatus 20, charging station 308 supplies electrical power to patient support apparatus 20. In some embodiments, charging station 308 may include any of the same features, functions, and/or structures as the facility interface 300 and tether T disclosed in commonly assigned U.S. provisional patent application Ser. No. 63/427,211 filed in 2023 and entitled SYSTEMS FOR ELECTRICALLY CONNECTING PATIENT SUPPORT APPARATUSES TO FACILITIES, the complete disclosure of which is incorporated herein by reference. Charging station 308 may take on still other forms.

Charging station 308 may include an indicator 310 that provides indications about one or more aspects of charging station 308. In the sample shown in FIG. 12, indicator 310 is positioned separately from the wall attachment portion where cable 314 attaches to the healthcare facility wall. However, it will be understood that indicator 310 may be integrated into the wall attachment portion, or it may be positioned in still other locations. Indicator 310 may comprise one or more lights, a sound emitter (e.g. speaker, buzzer, beeper, etc.), a display, a keypad, and/or other structures.

In some embodiments of patient support apparatus 20, controller 140 is configured to send a notification message to charging station 308 when controller 140 detects that patient support apparatus 20 is within a threshold distance of (i.e. within a particular volume of space 152) charging station 308 and cable 312 is not connected to patient support apparatus 20. The notification message instructs charging station 308 to activate indicator 310 so that a nearby caregiver sees and/or hears an indication reminding them to couple cable 314 to patient support apparatus 20.

In some embodiments, controller 140 is additionally, or alternatively, adapted to issue two different types of notifications when cord 314 is not coupled to patient support apparatus 20, depending upon the presence or absence of a caregiver within the vicinity of patient support apparatus 20. In such embodiments, controller 140 may be configured to issue a local notification when a caregiver is positioned within the vicinity of patient support apparatus 20 and to issue a remote notification when a caregiver is not positioned within the vicinity of patient support apparatus 20. The local notification includes doing one or more of the following on patient support apparatus 20 and/or on indicator 310 of charging station 308: emitting a sound, illuminating a light, flashing a light, and/or displaying information on a display. The remote notification includes one or more of the following: sending a notification message to server 84 via network transceiver 96, which then forwards the notification to one or more badges 142 and/or electronic devices 98 associated with a selected caregiver, or sending a notification message directly to a badge 142 and/or electronic device 98 that is within a threshold distance of patient support apparatus 20 (e.g. in the hallway). The direct sending of a notification message to a badge 142 and/or electronic device 98 may be carried out using one or more of the UWB transceivers 132 and/or using Bluetooth transceiver 128.

Controller 140 is configured to also switch the methods of notification in response to changing positions of a caregiver. For example, if controller 140 detects a nearby caregiver (say within a volume of space that only encompasses the patient's room, or a portion thereof), and it detects a charging station within the threshold distance that is not coupled to patient support apparatus 20, controller 140 will issue a local notification intended to remind the caregiver within that room to plug cord 314 into patient support apparatus 20. However, if the caregiver fails to plug cord 314 into patient support apparatus 20 and then leaves the room in which the patient is positioned, controller 140 may be configured to automatically terminate the local notification (so as to not potentially annoy a patient support apparatus 20) and issue a remote notification. The remote notification is intended to provide notification to the caregiver, or another authorized individual, of the need to connect cord 314 to patient support apparatus 20.

In all of the situations where controller 140 sends a notification to a badge 142 or electronic device 98 that is positioned within a volume of space 152 that extends out into the hallway, the notification may be intended to be received by any caregiver's badge 142 or electronic device 98, regardless of whether that particular caregiver is assigned to care for the particular patient assigned to patient support apparatus 20. By doing this, caregivers who happen to walk by, or near, a room in which patient support apparatus 20 has detected an undesired condition, will get a notification of the undesired condition via their badge 142 and/or electronic device 98. In this manner, caregivers can be automatically notified of undesired conditions that they are nearby to. Such automatic notifications to nearby personnel can also, or alternatively, be carried out by controller 140 sending a message to patient support apparatus server 84 via network transceiver 96, which then determines what caregivers are positioned near to patient support apparatus 20, and routes the notification to that (or those) particular caregiver(s) who are close to the patient support apparatus 20 that has detected the undesired condition.

In some embodiments, after determining that the relative location of one or more of devices 100 and/or badges 142 are currently positioned within a corresponding space volume 152, controller 140 forwards a message to patient support apparatus server 84 indicating that these devices (whose IDs are included in the message) are currently located in the same room as patient support apparatus 20. Patient support apparatus server 84 may forward this information to one or more of electronic devices 98, thereby enabling authorized individuals to track the location of pieces of equipment (including badges 142) within the healthcare facility. Controller 140 may therefore, in addition to determining the location of UWB-equipped devices 100 (and badges 142) for purposes of communicating data to/from those devices, also determine the location of those devices 100 (or badges 142) for tracking purposes. Patient support apparatus 20 therefore can facilitate the tracking of a variety of equipment used within the healthcare facility for inventory management purposes and/or for performing timely service on the equipment.

In some embodiments, UWB transceivers 126, 126a, 132, 158, and 178 (FIGS. 5 & 7) may operate in the same manner as, and include any of the same functions as, the anchors and pseudo-anchors disclosed in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which has already been incorporated herein by reference. In some embodiments, locator units 60 may also be configured to determine the location of a device (e.g. device 100, badge 142) in any of the manners disclosed in commonly assigned U.S. patent application Ser. No. 63/132,514 filed Dec. 31, 2020, by inventors Alexander Bodurka et al. and entitled PATIENT SUPPORT APPARATUS AND MEDICAL DEVICE NETWORKS, and in commonly assigned U.S. patent application Ser. No. 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosures of both of which are incorporated herein by reference.

It will also be understood that, although FIG. 5 illustrates badge 142 as including a UWB transceiver 158, patient support apparatus 20 and patient support apparatus server 84 may be configured to implement any of the functions described herein using badges 142 that do not include a UWB transceiver 158. In such situations, patient support apparatus server 84 determines whether to associate a badge 142 with a particular patient support apparatus 20 based upon the location information that may be independently gathered by badge server 94 using conventional WiFi triangulation techniques (or other conventional location determination techniques). Although the location technology utilized by badge server 94 may not be as precise as the UWB location technology described herein, patient support apparatus server 84 may be configured to use this location information to determine whether to associate and disassociate non-UWB equipped badges 142 with patient support apparatuses 20.

Display device 56 (FIG. 5) includes a display controller 192 and a display 194. Display device 56 differs from the other devices 100 and/or badges 142 discussed herein in that it does not need to include a UWB transceiver, such as is found in these other devices. This is because patient support apparatus 20 is not configured to determine the location of display device 56. Instead, controller 140 of patient support apparatus 20 is configured to use UWB transceivers 132 to determine the location of the locator unit 60 to which the display device 56 is coupled. If the patient support apparatus 20 (or a reference point on it) and locator unit 60 are not both positioned within the space volume 152a, the controller 140 will not send data to be displayed on display device 56 to the locator unit 60. If the locator unit 60 and patient support apparatus 20 (or a reference point on it) are both positioned within the space volume 152a, controller 140 is configured to be able to send data to be displayed on the coupled display device 56 by forwarding the data to be displayed to the associated locator unit 60, which then forwards it to display device 56 via video port 120 and video cable 110. In some embodiments, controller 140 forwards the data to be displayed on display device 56 to the associated locator unit 60 by using Bluetooth transceiver 128, while in other embodiments it may use a UWB transceiver 132, or another type of transceiver. The forwarding of data to be displayed on display device 56 is a direct forwarding of data from patient support apparatus 20 to locator unit 60, and from there to display device 56, with no other intermediaries, in at least some embodiments.

Because display device 56 need not include a UWB transceiver, display device 56 may be a conventional television, computer monitor, or other conventional device, that is capable of displaying the video signals that are transmitted over video cable 110. In addition, it is not necessary for display device 56 to execute any specialized software app that authenticates communication with the coupled locator unit 60 and/or with patient support apparatus 20. Instead, once controller 140 determines that locator unit 60 is within space volume 152a (or vice versa), controller 140 need not perform any additional security protocols regarding display device 56. Indeed, controller 140 does not need to be able to perform any communications with display device 56 other that forwarding data to be displayed to the associated locator unit 60. In this manner, display device 56 need not know anything about locator unit 60 and/or patient support apparatus 20. It merely displays the video signal coming from video cable 110, and therefore does not need a software app that is specialized to patient support apparatus 20 and/or to locator unit 60 and/or to UWB communications.

It should also be noted that the display of data on display 52 and/or display device 56 from an associated device is carried out by patient support apparatus 20 automatically. That is, for example, when a user has configured patient support apparatus 20 to display data from a device 100 on a display device 56, controller 140 automatically forwards this data to the associated display device 56. For example, if device 100 is a UWB-tagged heart rate monitor and a patient coupled to device 100 is wheeled into a bay area of a patient room that includes a display device 56 coupled to a locator unit 60, controller 140 is configured to automatically start displaying the patient's heart rate information on that display device 56 as soon as patient support apparatus 20 completes the following two association processes: (1) the association between patient support apparatus 20 and the locator unit 60 to which display device 56 is coupled, and (2) the association between patient support apparatus 20 and the heart rate monitor. In this manner, the caregiver sees the patient's heart rate data displayed on the display device 56 within seconds after moving the patient support apparatus 20 into the bay area, and the caregiver doesn't need to connect any cables, press any buttons, or take any other actions, in order for the heart rate data to be displayed on display device 56. Similarly, when the patient support apparatus 20 moves out of the bay area, the display of the patient's data on the display device 56 automatically terminates. This automatic termination is carried out after patient support apparatus 20 moves outside of the space volume 152a associated with the locator unit 60 to which the display device 56 is connected. Once outside of this space volume 152a, controller 140 disassociates patient support apparatus 20 from that locator unit 60 and its associated display device 56, and once this disassociation occurs, it stops sending data to be displayed on display device 56.

As another example of the automatic display of device data on a display, if a caregiver approaches a patient support apparatus 20 while carrying a portable electronic device 98—such as a smart phone or tablet computer that includes a display, a UWB transceiver, and the appropriate software app—controller 140 is adapted to automatically start forwarding the data to the electronic device 98 for display on its screen. In this manner, a caregiver that brings his or her portable electronic device 98 within space volume 152b (or another space volume defined for such devices 98) can have that patient's heart rate data (and/or other data, such as from another device 100) automatically displayed on his/her device 98. When the caregiver moves his or her portable electronic device 98 outside of the space volume 152b, the display of that patient's data automatically ceases because controller 140 automatically disassociates patient support apparatus 20 from the portable electronic device 98 and stops sending it data to be displayed. If the caregiver then moves to another patient's patient support apparatus 20 and steps inside the space volume 152b of that patient support apparatus, he or she will automatically see the data from the devices 100 associated with that patient displayed on his or her portable electronic device 98.

In some embodiments, controller 140 may be configured to display data from a device associated with patient support apparatus 20, and/or process the data from the associated device, in any of the manners disclosed in commonly assigned U.S. patent application Ser. No. 63/306,279 filed Feb. 3, 2022, by inventors Madhu Sandeep Thota et al. and entitled COMMUNICATION SYSTEM FOR PATIENT SUPPORT APPARATUSES, the complete disclosure of which is incorporated herein by reference.

In some embodiments, patient support apparatus 20 is configurable by a user (e.g. via control panel 54a) to control what information, if any, patient support apparatus 20 will send to an associated display device 56, to patient support apparatus server 84, and/or to EMR server 92. Thus, a user can instruct patient support apparatus 20, for example, to send blood pressure readings, breathing rate readings, and pulse rate readings from devices 100 that are vital sign sensors (and that are associated with patient support apparatus 20) to EMR server 92, but not status data from a device 100 that doesn't generate data for recordation in EMR server 92, such as a badge 142, a recliner, a portable nurse call device, or still other devices. Patient support apparatus 20 may also be configured by the user to route data for displays 52 and/or 56 and/or one or more servers on network 80 in still other manners.

Locator units 60 are configured to send a message to patient support apparatuses 20 indicating whether or not a display device 56 (FIGS. 4-5) is coupled thereto via video port 120. In such embodiments, when controller 140 of patient support apparatus 20 receives the signal indicating that a display device 56 is coupled to an associated locator unit 60, it is configured to consult the customized user settings to determine what data, if any, to send to that locator unit 60 for forwarding to the display device 56 coupled to video port 120. It bears noting that, when patient support apparatus 20 associates itself with a particular locator unit 60 that has a display device 56 coupled to its video port 120, it is not necessary for the display device 56 to be positioned within space volume 152a in order for controller 140 to send data to be displayed on the display device 56. Instead, controller 140 is configured to only check whether the locator unit 60 is associated with patient support apparatus 20 or not. If it is, controller 140 may send (depending upon the user settings) data to the locator unit 60 for forwarding to the display device 56. If locator unit 60 is not associated with patient support apparatus 20, then controller 140 does not send any data to the locator unit 60 for displaying on display device 56.

In addition to allowing a user to customize what data, if any, that is sent to a display device 56, controller 140 is further configured to allow a user to customize what data is sent to different types of display devices 56. Thus, controller 140 may be programmed to allow a user to send a first set of data to a display device 56 that is coupled to an associated locator unit 60 and a second set of data, different from the first set of data, to a different display, such as, for example, display 52 of patient support apparatus 20 and/or another display, such as, but not limited to, ones that are incorporated into electronic device 98 (e.g. conventional smart phones, laptop computers, tablet computers, smart TVs and/or smart monitors) that are either in communication with patient support apparatus server 84 and/or that are positioned within space volume 152, as determined by one or more UWB transceivers coupled to them that are adapted to communicate with UWB transceiver 132 of patient support apparatus 20. It will be appreciated, that in such cases, the conventional smart phone, laptop computer, tablet computer, or other type of display device will include a software app that oversees the UWB communications with patient support apparatus 20 and that controls the display of the data on the display (i.e. screen) of the display device. The software app includes one or more security features built into it that only allow authorized users to have this data displayed on the display device, thereby preventing unauthorized users from viewing this data on their own smart phone, laptop computer, tablet computer, etc. In some embodiments, controller 140 is configured to execute a security check protocol with the display device to authenticate the display device prior to sending it any data for displaying thereon.

Patient support apparatus 20 may include one or more screens that are displayable on display 52 that allow a user to customize the data from associated devices 100 that is shown on display 52 and/or display devices 56. In addition, patient support apparatus 20 is configured to allow the user to change the format and/or layout in which the selected data is to be displayed.

Although the foregoing description has primarily indicated that controller 140 determines whether to associate or disassociate patient support apparatus 20 with one or more devices 100, badges 142, and/or locator units 60, it will be understood that the association and/or disassociation process may alternatively, partially, and/or additionally, be carried out by patient support apparatus server 84. In such embodiments, controller 140 forwards relative position information regarding these devices to patient support apparatus server 84 so that server 84 can determine whether to associate a particular device with a particular patient support apparatus 20. In such embodiments, server 84 sends a message back to patient support apparatus 20 informing it of what devices it should be associated with, and what devices it should not be associated with.

In some embodiments, server 84 is configured to associate or disassociate devices 100 with specific patients. Server 84 may implement this patient association and disassociation process by storing in its memory data correlating specific device identifiers 166 or 180—which are transmitted to patient support apparatus 20 by their corresponding devices, and then forwarded by patient support apparatus 20 to server 84 via network transceiver 96—with specific patient support apparatus identifiers 130. In order to associate specific devices with a specific patient, patient support apparatus server 84 uses the location identifier 122 that it receives from a specific patient support apparatus 20 to determine the room location of the specific patient support apparatus 20. From this room location, server 84 receives data from ADT server 102 that identifies a specific patient for a specific room (or bay within a room). Server 84 is then able to correlate a specific device 100 or badge 142 (associated with the specific patient support apparatus 20 in that room) with a specific patient identifier because it knows the room (or bay) of the patient support apparatus 20, the specific IDs of the devices associated with that particular patient support apparatus 20, and the patient identifier associated with that particular room (or bay). After associating a device with a particular patient, server 84 is able to automatically retrieve data from a specific patient's medical records stored within EMR server 92 and/or to automatically send data from a particular device to the specific patient's medical records stored in EMR server 92.

Although FIG. 5 illustrates a single device 100 and a single badge 142, it will be understood that this is merely for illustration purposes. Multiple devices 100 and/or badges 142 may be within communication range of UWB transceivers 132 of patient support apparatus 20 and controller 140 of patient support apparatus 20 is adapted to determine the locations of those devices relative to a corresponding space volume 152b, c, d, etc. and, if they are positioned inside of the corresponding space volume 152, to automatically associate them with patient support apparatus 20 and make their data available for display, forward their data to patient support apparatus server 84, and/or take any of the other actions described herein.

In some embodiments, controller 140 is adapted to automatically associate itself with any one or more of the devices disclosed in commonly assigned U.S. patent application 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosure of which is incorporated herein by reference. Patient support apparatus 20 is configurable by a user to also, or alternatively, automatically display any of the data from these other types of devices on any of the displays 52 and/or associated display devices 56 when these devices are associated with patient support apparatus 20.

It will be understood that the frequency at which controller 140 repetitively determines the relative location of devices 100, locator unit 60, and badges 142 may vary in different embodiments. In some embodiments, this occurs multiple times a second. In other embodiments, this occurs less frequently than once a second. In still other embodiments, the frequency at which controller 140 determines the relative position of a device to patient support apparatus 20 may vary according to the device, the current location of patient support apparatus 20, the movement state of patient support apparatus 20 (i.e. whether it is currently moving or stationary), the brake state of patient support apparatus 20 (i.e. whether the brake 300 is on or off), and/or according to one or more other factors.

Controller 140 is configured to use the association status of a linked locator unit 60 (FIG. 4) to determine where to send data to for forwarding to the adjacent communication outlet 64, as well as to determine which linked locator unit 60 it will accept data from that originated from communication outlet 64. The associated linked locator unit 60 is the linked locator unit 60 that patient support apparatus 20 will send the patient's voice signals to for forwarding to nurse call system 70. It is also the linked locator unit 60 that controller 140 will send television commands to when a patient onboard patient support apparatus 20 activates one or more of the television controls 50l-50r. Similarly, it is the linked locator unit 60 that controller 140 will send light commands to when a patient onboard patient support apparatus 20 activates one or more or the reading or room light controls 50s or 50t. The linked locator unit 60 that patient support apparatus 20 associates itself with is also the linked locator unit 60 that patient support apparatus 20 will receive audio signals from and direct to its onboard speaker(s). Such audio signals may correspond to voice signals from a remotely positioned nurse that are forwarded to the corresponding communication outlet 64 by way of nurse call system 70, or such audio signals may correspond to television audio signals that are routed from television 72 to communication outlet 64 by way of the one or more conductors 68.

FIG. 8 illustrates an overview of the communication system of the present disclosure, including various types of data that may be stored within different servers on a healthcare facility's computer network 80. As was noted previously, EMR server 92 stores the electronic medical records of individual patients, and this data may include individual care plans for each patient, such as shown in table 322. ADT server 102 stores data indicating the current location within the healthcare facility for each patient, such as is indicated in table 320. Badge server 94 stores badge IDs (e.g. identifiers 180), the healthcare workers associated with those badge IDs, and whether or not the healthcare worker is currently logged into the system (e.g. whether the worker is currently using the system or not), as indicated by table 326. Caregiver assignment server 104 stores the rooms—and/or bays within rooms—of the patients that each caregiver is assigned to care for, as shown in table 324. Patient support apparatus server 84 stores a variety of data, some of which is illustrated in patient support table 330 of FIG. 8.

Patient support table 330 includes a column of locator IDs 122, a column of rooms, a column of bed status information, a column indicating the presence or absence of caregivers, a column indicating what devices 100 are associated with each patient support apparatus 20, a column of patient support apparatus IDs 130, and a column of function identifiers that indicate what functions have been performed using each patient support apparatus 20. The locator IDs 122 are forwarded by each patient support apparatus 20 to patient support apparatus server 84 after patient support apparatus 20 associates itself with a particular locator unit 60. The rooms corresponding to each locator ID are determined automatically by patient support apparatus server 84 using its onboard memory, which includes the surveying information from the installation of the locator units 60 (which, in turn, identifies the rooms and/or bays of each locator unit 60).

Patient support apparatus server 84 determines whether a caregiver is currently positioned nearby to each patient support apparatus 20 based upon whether the caregivers' badge 142 is currently associated with that patient support apparatus 20 or not. In other words, when controller 140 associates patient support apparatus 20 with a badge 142, it forwards this association determination (and the badge ID 180) to patient support apparatus server 84, which uses the ID 180 to determine the caregiver's identity. As long as the badge 142 remains associated with patient support apparatus 20, patient support apparatus server 84 considers the caregiver to be present within the vicinity of the patient support apparatus 20. Once controller 140 disassociates a badge 142 from patient support apparatus 20, it also sends a message to patient support apparatus server 84 and patient support apparatus server 84 then concludes that the caregiver is no longer present within the vicinity of the patient support apparatus 20.

As shown in table 330 of FIG. 8, patient support apparatus server 84 also receives data from the device(s) 100 that are currently associated with each patient support apparatus 20, as shown in the column labeled “Assoc. Device Status.” As has been described herein, patient support apparatus server 84 may forward some or all of this data to EMR server 92, one or more electronic devices 98, one or more other devices, and/or store this data. Table 330 also includes the patient support apparatus IDs 130 of each patient support apparatus 20. Still further, table 330 includes a column containing data identifying what functions have been performed with each patient support apparatus 20. This functional data is sent by controller 140 to patient support apparatus server 84 in response to a user carrying out one or more functions of patient support apparatus 20, as was descried previously.

It will be understood that EMR server 92, badge server 94, caregiver assignment server 104, and ADT server 102 (FIGS. 4 and 8) may all be conventional and/or commercially available servers.

In some embodiments, when patient support apparatus 20 and/or server 84 associate a device 100 and/or badge 142 with a particular patient support apparatus 20, controller 140 and/or server 84 are configured to inform medical personnel (via electronic devices 98) that the device is associated with a particular patient support apparatus 20 and/or with a particular patient assigned to that patient support apparatus 20. In this manner, data from the device can be correlated with a particular patient, room location, and/or patient support apparatus 20. Patient support apparatus 20 may therefore be configured to automatically forward data from the associated medical devices to server 84 after the devices become associated with patient support apparatus 20. Such data may, in turn, be automatically forwarded by server 84 to EMR server 92 for entry into the corresponding patient's electronic medical record. In this manner, once controller 140 associates a device with the patient support apparatus 20, data from the device can be automatically recorded in that patient's particular electronic medical record without requiring the caregiver to either manually associate the device with the patient and/or with the patient support apparatus 20 assigned to that patient. In other words, because controller 140 automatically determines that the device is positioned within a corresponding space volume 152, it is not necessary for a caregiver to take any manual steps to ensure that data from the device is forwarded to the proper corresponding patient's electronic medical record because patient support apparatus 20, along with server 84, automatically determine the correct patient associated with that medical device.

In any of the embodiments disclosed herein, server 84 may be configured to additionally execute a caregiver assistance software application of the type described in the following commonly assigned patent applications: U.S. patent application Ser. No. 62/826,97, filed Mar. 29, 2019 by inventors Thomas Durlach et al. and entitled PATIENT CARE SYSTEM; U.S. patent application Ser. No. 16/832,760 filed Mar. 27, 2020, by inventors Thomas Durlach et al. and entitled PATIENT CARE SYSTEM; and/or PCT patent application serial number PCT/US2020/039587 filed Jun. 25, 2020, by inventors Thomas Durlach et al. and entitled CAREGIVER ASSISTANCE SYSTEM, the complete disclosures of which are all incorporated herein by reference. That is, server 84 may be configured to share with one or more electronic devices 98 any of the information shared with the electronic devices disclosed in these aforementioned patent applications. Thus, for example, server 84 may be configured to not only share the location of patient support apparatuses 20 (and any devices that may be associated with them) with electronic devices 98, but it may also forward patient data to electronic devices 98 from one or more associated devices 100 (e.g. vital sign data, infusion pump data, etc.), patient support apparatus status data (e.g. current siderail position, bed exit status, brake status, motion lockout status, height status, scale data, etc.) badge 142 data, and/or caregiver rounding data (e.g. when the last rounding was performed for a particular patient, when the next rounds are due, etc.).

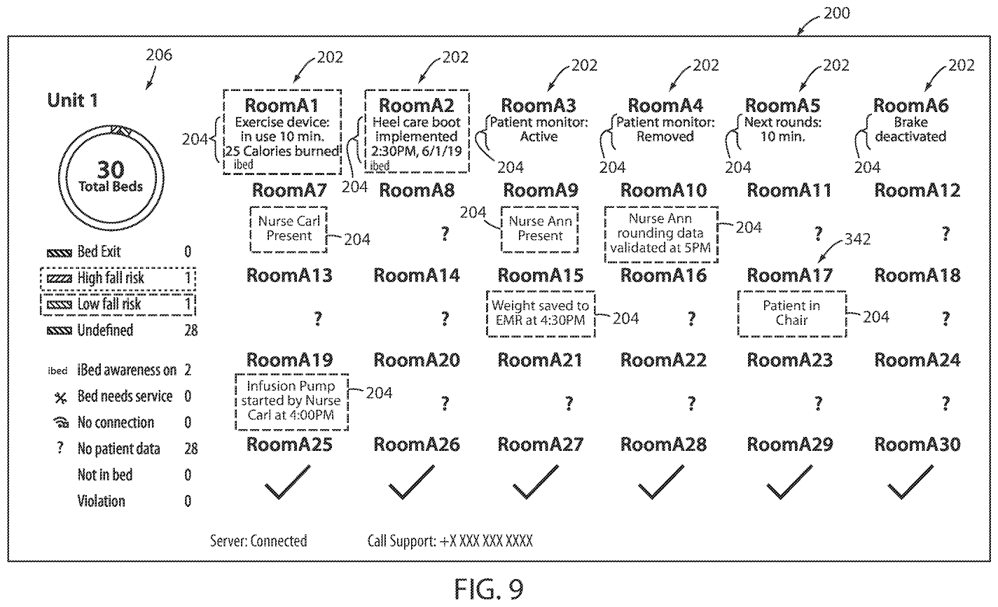

FIG. 9 illustrates one example of the type of data that patient support apparatus 20 may be configured to send to one or more electronic devices 98. More specifically, FIG. 9 illustrates one example of a dashboard screen 200 that patient support apparatus server 84 may be configured to display on one or more electronic devices 98. Dashboard screen 200 is a multi-room dashboard display that indicates the status of patient support apparatuses in a plurality of rooms of a healthcare facility, as well as information regarding the location and activities of healthcare workers with badges 142. The electronic device 98 on which dashboard screen 200 may be displayed includes a WiFi connection for communicating with an access point 82 of network 80, and thus is able to receive status data from patient support apparatus server 84.

Dashboard screen 200 includes a plurality of individual room numbers 202 and corresponding status messages 204 indicating one or more parameters about the status of the patient assigned to that room, the patient support apparatus 20 in that room, the caregiver assigned to the patient in that room, and/or any other workers with badges 142 who may be present in the room and/or who may have performed one or more functions on that patient support apparatus 20. The information that is displayable on screen 200 includes any information regarding the patient support apparatus 20 positioned in that room, as well as any information about badges 142, devices 100, and/or locator units 60 that are associated with that patient support apparatus. Thus, for example, as shown in FIG. 9, status messages 204 may indicate that an exercise device 100 has been in use for ten minutes and the patient has burned 25 calories (room A1), that a heel care boot 100 has been applied to a patient as of 2:30 PM on Jun. 1, 2019 (room A2); that a patient monitor 100 is active (room A3); that a patient monitor has been moved out of proximity of a patient support apparatus 20 (e.g. out of space volume 152b) (room A4); that a caregiver is expected to perform a rounding duty for a patient within a specific time period (room A5); that brake 300 on patient support apparatus 20 is deactivated (room A6); that nurse Carl is currently present in rooms A7; that Nurse Ann is currently present in room A9; that Nurse Ann's rounding data for room A10 was validated at 5 PM; that the patient's weight in room A15 was sent to EMR server 92 at 4:30 PM; that the patient is currently sitting in a recliner in room A17 (where the recliner includes a patient presence sensor and is a UWB-equipped device 100); and that an infusion pump was started by Nurse Carl at 4 PM in room A19. Still other types of information may be displayed on dashboard screen 200, including additional details regarding the status messages 204. Such additional details may be displayed in response to, for example, a user tapping on, or otherwise selecting a particular one of the rooms 202 on screen 200.

Screen 200 may also include a summary status area 206 that lists certain collective data statistics about patient support apparatuses 20, devices 100, badges 142, and/or patients. For example, summary area 206 may indicate how many patient support apparatuses 20 are currently issuing an exit alert (detected by exit detection system 136); how many patients are classified as high or low fall risks (or whose fall risk categorization has not been completed); how many patient support apparatuses 20 have their onboard monitoring system 138 activated; how many patient support apparatuses 20 are in need of service; how many are not connected to network 80, and/or other data. As noted, tapping on, or otherwise selecting, any individual room 202 of screen 200 may cause the electronic device 98 to display additional information about the status of the patient, patient support apparatus 20, and/or any device(s) 100 and/or badges 142 that are positioned in that room.

In some embodiments, locator units 60 may include additional information stored therein that is shared with patient support apparatus 20 when patient support apparatus 20 becomes associated with the locator unit 60. Such additional information may include location information identifying the relative position of the locator unit 60 with respect to one or more other locator units 60 that are positioned nearby. Additionally or alternatively, the locator units 60 may include information regarding the thickness and/or materials of the wall 62 to which it is attached, wherein such information provides an indication to the patient support apparatus 20 of the amount of attenuation that UWB signals will likely experience when traveling through that wall. Additionally or alternatively, the locator units 60 may include information identifying their general location within the healthcare facility (e.g. room 400, bay A of room 302, hallway X, maintenance area Y, radiology department, emergency department, etc.) and/or information identifying a more specific location of the locator units 60 within the healthcare facility (e.g. a set of X,Y,Z coordinates in a frame of reference that includes all, or a portion of, the healthcare facility; a height on the wall 62, a distance from one or more landmarks and/or architectural features within the healthcare facility, and/or other more specific information). In some embodiments, patient support apparatus 20 is adapted to utilize this information to determine its location within the healthcare facility and/or to determine whether it is positioned on the same side of the wall 62 as a particular locator unit 60. In some embodiments, patient support apparatus 20 and/or locator units 60 include any of the same structures, functions, and/or features of any of the patient support apparatuses and/or wall units disclosed in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosure of which has already been incorporated herein by reference.

It will also be understood that patient support apparatuses 20 and/or server 84 need not include all of the functionality described herein. That is, in some embodiments, for example, patient support apparatuses 20 may be adapted to carry out the automatic validation of certain functions performed by a caregiver, but might not include the ability to automatically detect nearby caregivers and automatically notify them of undesired conditions, or vice versa. In general, the features and functions of patient support apparatus 20 described herein may be implemented individually by themselves in a patient support apparatus 20 or they may be combined together in any desired combination.

In some embodiments, electronic devices 98 may include one or more smart phones 98 and/or tablet computers that are adapted to execute a software application that communicates with server 84 via access points 82 and/or that communicates directly with patient support apparatus 20 via one or more of the UWB transceivers 132 and/or Bluetooth transceiver 128. In some of these embodiments, the electronic device 98 may execute a software application that is the same as, or similar to, the Vocera Edge software module available from Stryker Corporation of Kalamazoo, Michigan. In some such embodiments, the Vocera Edge software module may be modified to include any of the capabilities discussed herein when installed on a smart phone or tablet computer that includes ultra-wideband communication abilities.

It will be understood by those skilled in the art that the use of the term “transceiver” throughout this specification is not intended to be limited to devices in which a transmitter and receiver are necessarily within the same housing, or share some circuitry. Instead, the term “transceiver” is used broadly herein to refer to both structures in which circuitry is shared between the transmitter and receiver, and transmitter-receivers in which the transmitter and receiver do not share circuitry and/or a common housing. Thus, the term “transceiver” refers to any device having a transmitter component and a receiver component, regardless of whether the two components are a common entity, separate entities, or have some overlap in their structures.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1-16. (canceled)

17. A patient support apparatus system comprising a patient support apparatus and a server, wherein the patient support apparatus comprises:
(a) a support surface adapted to support a patient;
(b) a plurality of ultra-wideband transceivers;
(c) a network transceiver adapted to communicate with the server; and
(d) a controller adapted to use radio frequency (RF) communication between the plurality of ultra-wideband transceivers and a badge worn by a user to determine a position of the badge relative to the patient support apparatus; to receive a badge identifier from the badge; to determine if the badge is positioned inside or outside of a volume of space; and to transmit the badge identifier to the server using the network transceiver if the badge is inside the volume of space; and
wherein the server is adapted to use the badge identifier to determine an identity of the user associated with the badge.

18. The patient support apparatus system of claim 17 wherein the server is further configured to transmit the identity of the user to a second server and to automatically log in the user to the second server.

19. The patient support apparatus system of claim 18 wherein the second server maintains an association between badge identifiers and user identities.

20. The patient support apparatus system of claim 17 wherein the patient support apparatus further comprises a first control, wherein the first control performs a first function when activated by the user, and wherein the controller is further adapted to transmit a validation message to the server validating that the first function was performed by the user if the badge is positioned inside the volume of space when the first control is activated.

21. The patient support apparatus system of claim 20 wherein the server is further adapted to transmit validation information to an electronic medical records server in response to receiving the validation message.

22. The patient support apparatus system of claim 20 wherein the first function is one or more of the following: changing a state of an exit detection system onboard the patient support apparatus; measuring a weight of the patient; performing a mattress therapy on the patient; changing a state of a mattress; changing a state of a brake onboard the patient support apparatus; changing a state of a siderail onboard the patient support apparatus; changing a height of the support surface; changing a reminder provided by the controller; zeroing a scale onboard the patient support apparatus; configuring a component onboard the patient support apparatus; changing a setting of the patient support apparatus; changing information stored in the patient support apparatus; or performing maintenance work on the patient support apparatus.

23. The patient support apparatus system of claim 17 wherein the patient support apparatus further comprises a first control, wherein the first control performs a first function when activated by the user, and wherein the controller is further adapted to transmit a function identifier to the server if the badge is positioned inside the volume of space when the first control is activated, wherein the function identifier identifies the first function.

24. The patient support apparatus system of claim 23 wherein the first function is one or more of the following: changing a state of an exit detection system onboard the patient support apparatus; measuring a weight of the patient; performing a mattress therapy on the patient; changing a state of a mattress; changing a state of a brake onboard the patient support apparatus; changing a state of a siderail onboard the patient support apparatus; changing a height of the support surface; changing a reminder provided by the controller; zeroing a scale onboard the patient support apparatus; configuring a component onboard the patient support apparatus; changing a setting of the patient support apparatus; changing information stored in the patient support apparatus; or performing maintenance work on the patient support apparatus.

25. The patient support apparatus system of claim 17 wherein the controller is further adapted to use RF communications between the plurality of ultra-wideband transceivers and a fixed locator to determine a position of the fixed locator relative to the patient support apparatus; to receive a locator identifier from the fixed locator; to determine if the patient support apparatus is within a threshold distance of the fixed locator; and to transmit the locator identifier to the server if the patient support apparatus is within the threshold distance of the fixed locator.

26. The patient support apparatus system of claim 25 wherein the server is adapted to use both the badge identifier and the locator identifier to determine the identity of the user associated with the badge.

27. The patient support apparatus system of claim 25 wherein the server is adapted to transmit at least a portion of a care plan to the patient support apparatus in response to receiving the badge identifier.

28-36. (canceled)

37. A patient support apparatus system comprising a patient support apparatus and a server, wherein the patient support apparatus comprises:
(a) a support surface adapted to support a patient;
(b) a plurality of ultra-wideband transceivers;
(c) a network transceiver adapted to communicate with the server; and
(d) a controller adapted to use radio frequency (RF) communication between the plurality of ultra-wideband transceivers and a badge worn by a user to determine a position of the badge relative to the patient support apparatus; to use RF communications between the plurality of ultra-wideband transceivers and a fixed locator to determine a position of the fixed locator relative to the patient support apparatus; to receive a badge identifier from the badge; to receive a locator identifier from the fixed locator; to determine if the badge is positioned inside or outside of a volume of space; to determine if the patient support apparatus is within a threshold distance of the fixed locator; to transmit the badge identifier to the server using the network transceiver if the badge is inside the volume of space; and to transmit the locator identifier to the server if the patient support apparatus is within the threshold distance of the fixed locator; and
wherein the server is adapted to use the locator identifier to determine an identity of the patient, and to automatically transmit at least a portion of a care plan to the patient support apparatus in response to the badge being inside the volume of space.

38. The patient support apparatus system of claim 37 wherein the controller is further adapted to display the at least a portion of the care plan on a display coupled to the patient support apparatus when the badge is positioned within the volume of space, and to automatically cease displaying the at least a portion of the care plan on the display in response to the badge moving outside the volume of space.

39. The patient support apparatus system of claim 38 wherein the server is further adapted to use the patient identity to determine the at least a portion of the care plan.

40. The patient support apparatus system of claim 39 wherein the server is adapted to communicate with an electronic medical records server to determine the at least a portion of the care plan.

41-47. (canceled)

48. The patient support apparatus system of claim 37 wherein the server is adapted to determine a room identifier in which the patient support apparatus is positioned by using the locator identifier, and to use the room identifier to determine the patient identity.

49. The patient support apparatus system of claim 37 wherein the controller is further adapted to determine whether the badge identifier belongs a first class of users or a second class of users and, if the badge identifier belongs to the first class, the controller is further adapted to automatically issue a first reminder to the user if the badge identifier belongs to the first class and the badge moves outside of the volume of space before a first function on the patient support apparatus has been completed.

50. The patient support apparatus system of claim 49 wherein the first function includes at least one of: zeroing of a scale system onboard the patient support apparatus or plugging a power cord of the patient support apparatus into an electrical outlet.

51. The patient support apparatus system of claim 49 the controller is further adapted to automatically issue a second reminder to the user if the badge identifier belongs to the second class and the badge moves outside of the volume of space before a second function of the patient support apparatus has been completed, the second function being different from the first function.

52. (canceled)

53. The patient support apparatus system of claim 37 wherein the controller is further adapted to monitor a plurality of conditions of the patient support apparatus, to determine if any of the conditions are not in a desired state, and to send a message to the server if any of the conditions are not in a desired state, and wherein the server is adapted to monitor a position of a plurality of badges and, in response to receiving the message, to automatically determine which badge of the plurality of badges is closest to the patient support apparatus and to send a second message to the badge closest to the patient support apparatus.

54-174. (canceled)
